(19) United States
(12) Patent Application Publication
(10) Pub. No.: US 2017/0281769 A1
(43) Pub. Date: Oct. 5, 2017

(54) STABLE ANTI-IL-4Ra FORMULATION

(71) Applicant: MedImmune Limited, Cambridge (GB)

(72) Inventors: Per-Olof Fredrik Eriksson, Lund (SE); Karin Von Wachenfeldt, Lund (SE); Suzanne Cohen, Cambridge (GB); Claire Louise Dobson, Cambridge (GB); Deborah Louise Lane, Cambridge (GB); Katrina Day, Cambridge (GB)

(21) Appl. No.: 15/508,647

(22) PCT Filed: Sep. 2, 2015
(86) PCT No.: PCT/EP2015/070091

§ 371 Date: Mar. 3, 2017

Related U.S. Application Data
(60) Provisional application No. 62/045,338, filed on Sep. 3, 2014.

(51) Int. Cl. A61K 39/395; A61K 47/22; A61K 47/26; C07K 16/28; A61K 47/18

(52) U.S. Cl. CPC A61K 39/39591; C07K 16/2866; A61K 47/183; A61K 47/26; A61K 47/22; C07K 2317/56; C07K 2317/565; C07K 2317/567

(57) ABSTRACT

The present invention relates to a stable, low viscosity antibody formulation, wherein the formulation comprises a high concentration of anti-IL4R antibody. In some embodiments, the invention relates in general to a stable antibody formulation comprising about 100 mg/mL to about 200 mg/mL of an antibody or fragment thereof that specifically binds human interleukin-4 receptor alpha (hIL-4Rα), about 50 mM to about 400 mM of a viscosity modifier; about 0.002% to about 0.2% of a non-ionic surfactant; and a formulation buffer. In some embodiments, the formulation buffer is essentially free of phosphate. In some embodiments, the invention is directed to a container, dosage form and/or kit. In some embodiments, the invention is directed to a method of making and using the stable antibody formulation.

This application claims benefit of U.S. Provisional Patent Application No. 62/045,338, filed Sep. 3, 2014, the disclosure of which is incorporated by reference herein in its entirety.

This application incorporates by reference a Sequence Listing submitted with the application via EFS-Web as a test filed entitled “IL4R300P1” created on May 13, 2013 and having a size of 214 kilobytes.

### BACKGROUND OF THE INVENTION

### Field of the Invention

The present invention relates to a stable, low viscosity antibody formulation, wherein the formulation comprises a high concentration of anti-IL4R antibody. In some embodiments, the invention relates in general to a stable antibody formulation comprising about 100 mg/mL to about 200 mg/mL of an antibody or fragment thereof that specifically binds human interleukin-4 receptor alpha (hIL-4Rα), about 50 mM to about 400 mM of a viscosity modifier; about 0.002% to about 0.2% of a non-ionic surfactant; and a formulation buffer. In some embodiments, the formulation buffer is essentially free of phosphate. In some embodiments, the invention is directed to a container, dosage form and/or kit. In some embodiments, the invention is directed to a method of making and using the stable antibody formulation.

### Background

Antibodies have been used in the treatment of various diseases and conditions due to their specificity of target recognition, thereby generating highly selective outcomes following systemic administration. In order for antibodies to remain effective, they must maintain their biological activity during their production, purification, transport and storage. New production and purification techniques have been developed to provide for large amounts of highly purified monoclonal antibodies to be produced. However, challenges still exist to stabilize these antibodies for transport and storage, and yet even more challenges exist to provide the antibodies in a dosage form suitable for administration.

Denaturation, aggregation, contamination, and particle formation can be significant obstacles in the formulation and storage of antibodies. Due to the wide variety of antibodies, there are no universal formulations or conditions suitable for storage of all antibodies. Optimal formulations of one antibody are often specific to that antibody. Additionally, antibody formulations may need to be further tailored to a specific antibody depending on the concentration of the antibody, and/or a desired physical property, e.g., viscosity, of the antibody formulation. Antibody storage formulations are often a significant part of the research and development process for a commercial antibody. Thus, a need exists to provide stable, aqueous antibody formulations that can overcome the challenges associated with transport and storage.

Citation or discussion of a reference herein shall not be construed as an admission that such is prior art to the present invention.

### SUMMARY OF THE INVENTION

The present invention relates to a stable, low viscosity antibody formulation, wherein the formulation comprises a high concentration of anti-IL4R antibody. In some embodiments, the invention relates in general to a stable antibody formulation comprising about 100 mg/mL to about 200 mg/mL of an antibody or fragment thereof that specifically binds human interleukin-4 receptor alpha (hIL-4Rα), about 50 mM to about 400 mM of a viscosity modifier; about 0.002% to about 0.2% of a non-ionic surfactant; and a formulation buffer.

In some embodiments, the invention is directed to a stable antibody formulation comprising: about 100 mg/mL to about 200 mg/mL of an antibody or fragment thereof that specifically binds human interleukin-4 receptor alpha (hIL-4Rα), wherein:
(I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
   HCDR1 has amino acid sequence SEQ ID NO: 193;
   HCDR2 has amino acid sequence SEQ ID NO: 194;
   HCDR3 has amino acid sequence SEQ ID NO: 195;
   LCDR1 has amino acid sequence SEQ ID NO: 198;
   LCDR2 has amino acid sequence SEQ ID NO: 199; and
   LCDR3 has amino acid sequence SEQ ID NO: 200;
(II)
   the HCDR1 has amino acid sequence SEQ ID NO: 363;
   the HCDR2 has amino acid sequence SEQ ID NO: 364;
   the HCDR3 has amino acid sequence SEQ ID NO: 365;
   the LCDR1 has amino acid sequence SEQ ID NO: 368;
   the LCDR2 has amino acid sequence SEQ ID NO: 369; and
   the LCDR3 has amino acid sequence SEQ ID NO: 370;
OR
   the HCDR1 has amino acid sequence SEQ ID NO: 233;
   the HCDR2 has amino acid sequence SEQ ID NO: 234;
   the HCDR3 has amino acid sequence SEQ ID NO: 235;
   the LCDR1 has amino acid sequence SEQ ID NO: 238;
   the LCDR2 has amino acid sequence SEQ ID NO: 239; and
   the LCDR3 has amino acid sequence SEQ ID NO: 240;
(III) the antibody comprises a VH domain wherein:
   i. the VH domain has amino acid sequence SEQ ID NO: 192;
   ii. the VH domain has amino acid sequence SEQ ID NO: 362; or
   iii. the VH domain has amino acid sequence SEQ ID NO: 232; and,
      wherein the VH domain comprises one or more amino acid substitutions at the following residues within the framework regions, using the standard numbering of Kabat:
11, 12 in HFW1;
37, 48 in HFW2;
68, 84, 85 in HFW3; or
105, 108, 113 in HFW4;
(IV) the antibody comprises a VL domain wherein:
   i. the VL domain has amino acid sequence SEQ ID NO: 197;
   ii. the VL domain has amino acid sequence SEQ ID NO: 367; or
   iii. the VL domain has amino acid sequence SEQ ID NO: 237; and,
      wherein the VL domain comprises one or more amino acid substitutions at the following residues within the framework regions, using the standard numbering of Kabat:
1, 2, 3, 9 in LFW1;
38, 42 in LFW2; or
58, 65, 66, 70, 74, 85, 87 in LFW3;
OR
(V) wherein the antibody or fragment thereof comprises a VH and a VL domain wherein:
   i. the VH domain has amino acid sequence SEQ ID NO: 192 and the VL domain has amino acid sequence SEQ ID NO: 197;
   ii. the VH domain has amino acid sequence SEQ ID NO: 362 and the VL domain has amino acid sequence SEQ ID NO: 367; or
   iii. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3;
   or any combination of (I)-(V); and about 50 mM to about 400 mM of a viscosity modifier; about 0.002% to about 0.2% of a non-ionic surfactant; and a formulation buffer.

In some embodiments, the invention is directed to a stable antibody formulation comprising: about 100 mg/mL to about 200 mg/mL of an antibody or fragment thereof that specifically binds human interleukin-4 receptor alpha (hIL-4Rα), wherein:
(I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
   HCDR1 has amino acid sequence SEQ ID NO: 193;
   HCDR2 has amino acid sequence SEQ ID NO: 194;
   HCDR3 has amino acid sequence SEQ ID NO: 195;
   LCDR1 has amino acid sequence SEQ ID NO: 198;
   LCDR2 has amino acid sequence SEQ ID NO: 199; and
   LCDR3 has amino acid sequence SEQ ID NO: 200;
(II)
   the HCDR1 has amino acid sequence SEQ ID NO: 363;
   the HCDR2 has amino acid sequence SEQ ID NO: 364;
   the HCDR3 has amino acid sequence SEQ ID NO: 365;
   the LCDR1 has amino acid sequence SEQ ID NO: 368;
   the LCDR2 has amino acid sequence SEQ ID NO: 369; and
   the LCDR3 has amino acid sequence SEQ ID NO: 370;
OR
   the HCDR1 has amino acid sequence SEQ ID NO: 233;
   the HCDR2 has amino acid sequence SEQ ID NO: 234;
   the HCDR3 has amino acid sequence SEQ ID NO: 235;
   the LCDR1 has amino acid sequence SEQ ID NO: 238;
   the LCDR2 has amino acid sequence SEQ ID NO: 239; and
   the LCDR3 has amino acid sequence SEQ ID NO: 240;
(III) the antibody comprises a VH domain wherein:
   i. the VH domain has amino acid sequence SEQ ID NO: 192;
   ii. the VH domain has amino acid sequence SEQ ID NO: 362; or
   iii. the VH domain has amino acid sequence SEQ ID NO: 232; and,
      wherein the VH domain comprises one or more amino acid substitutions at the following residues within the framework regions, using the standard numbering of Kabat:
11, 12 in HFW1;
37, 48 in HFW2;
68, 84, 85 in HFW3; or
105, 108, 113 in HFW4;
(IV) the antibody comprises a VL domain wherein:
   i. the VL domain has amino acid sequence SEQ ID NO: 197;
   ii. the VL domain has amino acid sequence SEQ ID NO: 367; or
   iii. the VL domain has amino acid sequence SEQ ID NO: 237; and,
      wherein the VL domain comprises one or more amino acid substitutions at the following residues within the framework regions, using the standard numbering of Kabat:
1, 2, 3, 9 in LFW1;
38, 42 in LFW2; or
58, 65, 66, 70, 74, 85, 87 in LFW3;
OR
(V) wherein the antibody or fragment thereof comprises a VH and a VL domain wherein:
   i. the VH domain has amino acid sequence SEQ ID NO: 192 and the VL domain has amino acid sequence SEQ ID NO: 197;
   ii. the VH domain has amino acid sequence SEQ ID NO: 362 and the VL domain has amino acid sequence SEQ ID NO: 367; or
   iii. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3;
   or any combination of (I)-(V); and about 50 mM to about 400 mM arginine; about 0.002% to about 0.2% polysorbate 80; and about 10 to about 40 mM L-histidine/L-histidine hydrochloride.

In some embodiments, the invention is directed to a stable antibody formulation comprising: about 100 mg/mL to about 200 mg/mL of an antibody or fragment thereof that specifically binds human interleukin-4 receptor alpha (hIL-4Rα), wherein:
(I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
   HCDR1 has amino acid sequence SEQ ID NO: 193;
   HCDR2 has amino acid sequence SEQ ID NO: 194;
   HCDR3 has amino acid sequence SEQ ID NO: 195;
   LCDR1 has amino acid sequence SEQ ID NO: 198;
   LCDR2 has amino acid sequence SEQ ID NO: 199; and
   LCDR3 has amino acid sequence SEQ ID NO: 200;
(II)
   the HCDR1 has amino acid sequence SEQ ID NO: 363;
   the HCDR2 has amino acid sequence SEQ ID NO: 364;
   the HCDR3 has amino acid sequence SEQ ID NO: 365;
   the LCDR1 has amino acid sequence SEQ ID NO: 368;
   the LCDR2 has amino acid sequence SEQ ID NO: 369; and
   the LCDR3 has amino acid sequence SEQ ID NO: 370;
OR
   the HCDR1 has amino acid sequence SEQ ID NO: 233;
   the HCDR2 has amino acid sequence SEQ ID NO: 234;
   the HCDR3 has amino acid sequence SEQ ID NO: 235;
   the LCDR1 has amino acid sequence SEQ ID NO: 238;
   the LCDR2 has amino acid sequence SEQ ID NO: 239; and
   the LCDR3 has amino acid sequence SEQ ID NO: 240;
(III) the antibody comprises a VH domain wherein:
   i. the VH domain has amino acid sequence SEQ ID NO: 192;
   ii. the VH domain has amino acid sequence SEQ ID NO: 362; or
   iii. the VH domain has amino acid sequence SEQ ID NO: 232; and,
      wherein the VH domain comprises one or more amino acid substitutions at the following residues within the framework regions, using the standard numbering of Kabat:
11, 12 in HFW1;
37, 48 in HFW2;
68, 84, 85 in HFW3; or
105, 108, 113 in HFW4;
(IV) the antibody comprises a VL domain wherein:
   i. the VL domain has amino acid sequence SEQ ID NO: 197;
   ii. the VL domain has amino acid sequence SEQ ID NO: 367; or
   iii. the VL domain has amino acid sequence SEQ ID NO: 237; and,
      wherein the VL domain comprises one or more amino acid substitutions at the following residues within the framework regions, using the standard numbering of Kabat:
1, 2, 3, 9 in LFW1;
38, 42 in LFW2; or
58, 65, 66, 70, 74, 85, 87 in LFW3;
OR
(V) wherein the antibody or fragment thereof comprises a VH and a VL domain wherein:
   i. the VH domain has amino acid sequence SEQ ID NO: 192 and the VL domain has amino acid sequence SEQ ID NO: 197;
   ii. the VH domain has amino acid sequence SEQ ID NO: 362 and the VL domain has amino acid sequence SEQ ID NO: 367; or
   iii. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3;
   or any combination of (I)-(V); and about 190 mM arginine; about 0.04% polysorbate 80; and about 25 mM L-histidine/L-histidine hydrochloride.

In some embodiments, the invention is directed to a pharmaceutical unit dosage form suitable for parenteral administration to a human which comprises any one of the antibody formulations described herein in a suitable container.

In some embodiments, the invention is directed to a kit comprising any antibody formulation described herein, a container as described herein, a unit dosage form as described herein, or a pre-filled syringe as described herein.

In some embodiments, the invention is directed to a method of producing a stable, aqueous antibody formulation, the method comprising:
A. purifying an antibody to about 100 mg/mL to about 200 mg/mL of an antibody or fragment thereof that specifically binds human interleukin-4 receptor alpha (hIL-4Rα), wherein:
   (I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
      HCDR1 has amino acid sequence SEQ ID NO: 193;
      HCDR2 has amino acid sequence SEQ ID NO: 194;
      HCDR3 has amino acid sequence SEQ ID NO: 195;
      LCDR1 has amino acid sequence SEQ ID NO: 198;
      LCDR2 has amino acid sequence SEQ ID NO: 199; and
      LCDR3 has amino acid sequence SEQ ID NO: 200;
   (II)
      the HCDR1 has amino acid sequence SEQ ID NO: 363;
      the HCDR2 has amino acid sequence SEQ ID NO: 364;
      the HCDR3 has amino acid sequence SEQ ID NO: 365;
      the LCDR1 has amino acid sequence SEQ ID NO: 368;
      the LCDR2 has amino acid sequence SEQ ID NO: 369; and
      the LCDR3 has amino acid sequence SEQ ID NO: 370;
   OR
      the HCDR1 has amino acid sequence SEQ ID NO: 233;
      the HCDR2 has amino acid sequence SEQ ID NO: 234;
      the HCDR3 has amino acid sequence SEQ ID NO: 235;
      the LCDR1 has amino acid sequence SEQ ID NO: 238;
      the LCDR2 has amino acid sequence SEQ ID NO: 239; and
      the LCDR3 has amino acid sequence SEQ ID NO: 240;
   (III) the antibody comprises a VH domain wherein:
      the VH domain has amino acid sequence SEQ ID NO: 192;
      the VH domain has amino acid sequence SEQ ID NO: 362; or
      the VH domain has amino acid sequence SEQ ID NO: 232; and,
         wherein the VH domain comprises one or more amino acid substitutions at the following residues within the framework regions, using the standard numbering of Kabat:
11, 12 in HFW1;
37, 48 in HFW2;
68, 84, 85 in HFW3; or
105, 108, 113 in HFW4;
   (IV) the antibody comprises a VL domain wherein:
      the VL domain has amino acid sequence SEQ ID NO: 197;
      the VL domain has amino acid sequence SEQ ID NO: 367; or
      the VL domain has amino acid sequence SEQ ID NO: 237; and,
         wherein the VL domain comprises one or more amino acid substitutions at the following residues within the framework regions, using the standard numbering of Kabat:
1, 2, 3, 9 in LFW1;
38, 42 in LFW2; or
58, 65, 66, 70, 74, 85, 87 in LFW3;
   OR
   (V) wherein the antibody or fragment thereof comprises a VH and a VL domain wherein:
      i. the VH domain has amino acid sequence SEQ ID NO: 192 and the VL domain has amino acid sequence SEQ ID NO: 197;
      ii. the VH domain has amino acid sequence SEQ ID NO: 362 and the VL domain has amino acid sequence SEQ ID NO: 367; or
      iii. the VH domain has amino acid sequence SEQ ID NO: 232 and the VL domain has amino acid sequence SEQ ID NO: 237; and,
         wherein the VH domain and VL domain comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3;
or any combination of (I)-(V); and
B. placing the isolated antibody in a stabilizing formulation to form the stable, aqueous antibody formulation, wherein the resulting stable, aqueous antibody formulation comprises:
   i. about 100 mg/mL to about 200 mg/mL of the antibody;
   ii. about 50 mM to about 400 mM of a viscosity modifier;
   iii. about 0.002% to about 0.2% of a non-ionic surfactant; and
   iv. a formulation buffer.

In some embodiments, the invention is directed to a method of treating a pulmonary disease or disorder in a subject, or inflammatory skin disorder, the method comprising administering a therapeutically effective amount of any one of the antibody formulations described herein.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 demonstrates that the addition of an ionic excipient such as Arginine-HCL or sodium chloride reduces the viscosity of an anti-IL4R antibody to <10 cP at 23° C.

FIG. 2 is a photograph of non-agitated Anti-hIL-4Rα at approximately 150 mg/ml in a formulation containing 25 mM Histidine/Histidine-HCL, 190 mM Arginine-HCL, pH 6.

FIG. 3 is a photograph of agitated Anti-hIL-4Rα at approximately 150 mg/ml in a formulation containing 25 mM Histidine/Histidine-HCL, 190 mM Arginine-HCL, pH 6.

FIG. 4 is a photograph of agitated Anti-hIL-4Rα at approximately 150 mg/ml in a formulation containing 25 mM Histidine/Histidine-HCL, 190 mM Arginine-HCL, pH 6, 0.01% polysorbate 80.

FIG. 5 is a photograph of Anti-hIL-4Rα at approximately 150 mg/ml in a formulation containing 25 mM Histidine/Histidine-HCL, 190 mM Arginine-HCL, pH 6. This sample has not been subjected to freeze thaw.

FIG. 6 is a photograph of Anti-hIL-4Rα at approximately 150 mg/ml in a formulation containing 25 mM Histidine/Histidine-HCL, 190 mM Arginine-HCL, pH 6. This sample has been subjected to 5× freeze thaw.

FIG. 7 is a photograph of Anti-hIL-4Rα approximately 150 mg/ml in a formulation containing 25 mM Histidine/Histidine-HCL, 190 mM Arginine-HCL, pH 6, 0.01% polysorbate 80. This sample has been subjected to 5× freeze thaw.

FIG. 8 is a scatter graph of total peak area absorbance (HPSEC) versus time at 40° C. for an anti-IL4R antibody formulation at (i) pH 5.5 (ii) pH 6.0, or (iii) pH 6.5.

FIG. 9 is a scatter graph of total peak area absorbance (HPSEC) versus time for an anti-IL4R antibody formulation stored at (i) 2-8° C. (ii) 25° C. or (iii) 40° C.

FIG. 10 is a scatter graph of percent total peak area reduction after 8 weeks at 40° C. (HPSEC) versus Tₘ1 for an anti-IL4R antibody formulation at (i) pH 5.5 (ii) pH 6 or (iii) pH 6.5.

FIG. 11 is a column chart for number of ≧10 μM particles/ml versus diluent for an anti-IL4R antibody formulation stored for 4 weeks (i) 2-8° C. (ii) 25° C. or (iii) 35° C. or (iv) 40° C.

FIG. 12 is a column chart for number of ≧10 μM particles/ml versus diluent for an anti-IL4R antibody formulation stored for 4 weeks at 40° C.

FIG. 13 shows the alignment of the VH domains of Antibodies 2-42 against Antibody 1 (split into sheets A, B, C, and D).

FIG. 14 shows the alignment of the VI domains of Antibodies 2-42 against Antibody 1 (split into sheets A, B, C, and D).

FIG. 15 shows the alignment of the VH domains of Antibodies 1-19 and 21-42 against Antibody 20 (split into sheets A, B, C, and D).

FIG. 16 shows the alignment of the VI domains of Antibodies 1-19 and 21-42 against Antibody 20 (split into sheets A, B, C, and D).

FIG. 17 shows samples containing >0.01% polysorbate 80 (PS 80) contained less visible particles after agitation than the lowest particle standard.

FIG. 18 shows the addition of >0.02% PS80 and <0.7% PS 80 in agitated samples is required to reduce the concentration of ≧10 μm particles to a level comparable to a sample that did not undergo agitation.

FIG. 19 shows samples containing >0.005% PS80 contained less visible particles after freeze thaw cycling relative to the lowest particle standard.

### DETAILED DESCRIPTION OF THE INVENTION

### Definitions

Before describing the present invention in detail, it is to be understood that this invention is not limited to specific compositions or process steps, as such can vary. It must be noted that, as used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A,” (alone) and “B” (alone) Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

It is understood that wherever embodiments are described herein with the language “comprising,” otherwise analogous embodiments described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The term “epitope” as used herein refers to a protein determinant capable of binding to a scaffold of the invention. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

The term “DNA” refers to a sequence of two or more covalently bonded, naturally occurring or modified deoxyribonucleotides.

A “protein sequence” or “amino acid sequence” means a linear representation of the amino acid constituents in a polypeptide in an amino-terminal to carboxyl-terminal direction in which residues that neighbor each other in the representation are contiguous in the primary structure of the polypeptide.

The term “nucleic acid” refers to any two or more covalently bonded nucleotides or nucleotide analogs or derivatives. As used herein, this term includes, without limitation, DNA, RNA, and PNA. “Nucleic acid” and “polynucleotide” are used interchangeably herein.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). The term “isolated” nucleic acid or polynucleotide refers to a nucleic acid molecule, DNA or RNA that has been removed from its native environment. For example, a recombinant polynucleotide encoding, e.g., a scaffold of the invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid can be or can include a regulatory element such as a promoter, ribosome binding site, or a transcription terminator.

By a “polypeptide” is meant any sequence of two or more amino acids linearly linked by amide bonds (peptide bonds) regardless of length, post-translation modification, or function. “Polypeptide,” “peptide,” and “protein” are used interchangeably herein. Thus, peptides, dipeptides, tripeptides, or oligopeptides are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. A polypeptide can be generated in any manner, including by chemical synthesis.

Also included as polypeptides of the present invention are fragments, derivatives, analogs, or variants of the foregoing polypeptides, and any combination thereof. Variants can occur naturally or be non-naturally occurring. Non-naturally occurring variants can be produced using art-known mutagenesis techniques. Variant polypeptides can comprise conservative or non-conservative amino acid substitutions, deletions, or additions. Also included as “derivatives” are those peptides that contain one or more naturally occurring amino acid derivatives of the twenty standard amino acids.

By “randomized” or “mutated” is meant including one or more amino acid alterations, including deletion, substitution or addition, relative to a template sequence. By “randomizing” or “mutating” is meant the process of introducing, into a sequence, such an amino acid alteration. Randomization or mutation can be accomplished through intentional, blind, or spontaneous sequence variation, generally of a nucleic acid coding sequence, and can occur by any technique, for example, PCR, error-prone PCR, or chemical DNA synthesis. The terms “randomizing”, “randomized”, “mutating”, “mutated” and the like are used interchangeably herein.

By a “cognate” or “cognate, non-mutated protein” is meant a protein that is identical in sequence to a variant protein, except for the amino acid mutations introduced into the variant protein, wherein the variant protein is randomized or mutated.

By “RNA” is meant a sequence of two or more covalently bonded, naturally occurring or modified ribonucleotides. One example of a modified RNA included within this term is phosphorothioate RNA.

The term “expression” as used herein refers to a process by which a gene produces a biochemical, for example, a scaffold of the invention or a fragment thereof. The process includes any manifestation of the functional presence of the gene within the cell including, without limitation, gene knockdown as well as both transient expression and stable expression. It includes without limitation transcription of the gene into one or more mRNAs, and the translation of such mRNAs into one or more polypeptides. If the final desired product is a biochemical, expression includes the creation of that biochemical and any precursors.

An “expression product” can be either a nucleic acid, e.g., a messenger RNA produced by transcription of a gene, or a polypeptide. Expression products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The term “vector” or “expression vector” is used herein to mean vectors used in accordance with the present invention as a vehicle for introducing into and expressing a desired expression product in a host cell. As known to those skilled in the art, such vectors can easily be selected from the group consisting of plasmids, phages, viruses and retroviruses. In general, vectors compatible with the instant invention will comprise a selection marker, appropriate restriction sites to facilitate cloning of the desired nucleic acid and the ability to enter and/or replicate in eukaryotic or prokaryotic cells.

The term “host cells” refers to cells that harbor vectors constructed using recombinant DNA techniques and encoding at least one expression product. In descriptions of processes for the isolation of an expression product from recombinant hosts, the terms “cell” and “cell culture” are used interchangeably to denote the source of the expression product unless it is clearly specified otherwise, i.e., recovery of the expression product from the “cells” means either recovery from spun down whole cells, or recovery from the cell culture containing both the medium and the suspended cells.

The terms “treat” or “treatment” as used herein refer to both therapeutic treatment and prophylactic or preventative measures, wherein the object is to prevent or slow down (lessen) an undesired physiological change or disorder in a subject, such as the progression of an inflammatory disease or condition. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

The term “treatment” also means prolonging survival as compared to expected survival if not receiving treatment. Those in need of treatment include those already with the condition or disorder as well as those prone to have the condition or disorder or those in which the condition or disorder is to be prevented.

The terms “subject,” “individual,” “animal,” “patient,” or “mammal” refer to any individual, patient or animal, in particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “target” refers to a compound recognized by a specific antibody of the invention. The terms “target” and “antigen” are used interchangeably herein. The term “specificity” as used herein, e.g., in the terms “specifically binds” or “specific binding,” refers to the relative affinity by which an antibody of the invention binds to one or more antigens via one or more antigen binding domains, and that binding entails some complementarity between one or more antigen binding domains and one or more antigens. According to this definition, an antibody of the invention is said to “specifically bind” to an epitope when it binds to that epitope more readily than it would bind to a random, unrelated epitope.

The term “affinity” as used herein refers to a measure of the strength of the binding of a certain antibody of the invention to an individual epitope.

The term “avidity” as used herein refers to the overall stability of the complex between a population of antibodies of the invention and a certain epitope, i.e., the functionally combined strength of the binding of a plurality of antibodies with the antigen. Avidity is related to both the affinity of individual antigen-binding domains with specific epitopes, and also the valency of the antibody of the invention.

The term “action on the target” refers to the binding of an antibody of the invention to one or more targets and to the biological effects resulting from such binding.

The term “immunoglobulin” and “antibody” comprises various broad classes of polypeptides that can be distinguished biochemically. Those skilled in the art will appreciate that heavy chains are classified as gamma, mu, alpha, delta, or epsilon. It is the nature of this chain that determines the “class” of the antibody as IgG, IgM, IgA IgG, or IgE, respectively. Modified versions of each of these classes are readily discernible to the skilled artisan. As used herein, the term “antibody” includes but not limited to an intact antibody, a modified antibody, an antibody VL or VL domain, a CH1 domain, a Ckappa domain, a Clambda domain, an Fc domain (see below), a CH2, or a CH3 domain.

As used herein, the term “modified antibody” includes synthetic forms of antibodies which are altered such that they are not naturally occurring, e.g., antibodies that comprise at least two heavy chain portions but not two complete heavy chains (as, e.g., domain deleted antibodies or minibodies); multispecific forms of antibodies (e.g., bispecific, trispecific, etc.) altered to bind to two or more antigens or to different epitopes of a single antigen). In addition, the term “modified antibody” includes multivalent forms of antibodies (e.g., trivalent, tetravalent, etc., antibodies that to three or more copies of the same antigen). (See, e.g., Antibody Engineering, Kontermann & Dubel, eds., 2010, Springer Protocols, Springer).

An antibody of the invention can be from any animal origin including birds and mammals. In some embodiments, the antibody of the methods of the invention are human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598 by Kucherlapati et al.

An antibody of the invention can include, e.g., native antibodies, intact monoclonal antibodies, polyclonal antibodies, multispecific antibodies (e.g., bispecific antibodies) formed from at least two intact antibodies, antibody fragments (e.g., antibody fragments that bind to and/or recognize one or more antigens), humanized antibodies, human antibodies (Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)). An antibody purified by the method of the invention can be recombinantly fused to a heterologous polypeptide at the N- or C-terminus or chemically conjugated (including covalently and non-covalently conjugations) to polypeptides or other compositions. For example, an antibody purified by the method of the present invention can be recombinantly fused or conjugated to molecules useful as labels in detection assays and effector molecules such as heterologous polypeptides, drugs, or toxins. See, e.g., PCT publications WO 92/08495; WO 91/14438; WO 89/12624; U.S. Pat. No. 5,314,995; and EP 396,387.

### IL-4Rα

IL-4Rα, is interleukin-4 receptor alpha. References to IL-4Rα are normally to human IL-4Rα unless otherwise indicated. A sequence of wild-type mature human IL-4Rα is deposited under Accession number P24394 (Swiss-Prot), which shows the full-length IL-4Rα including the signal peptide.

Cynomolgus IL-4Rα was sequenced in-house, the cDNA sequence of cynomolgus IL-4Rα is shown as SEQ ID NO: 455.

As described elsewhere herein, IL-4Rα may be recombinant, and/or may be either glycosylated or unglycosylated. IL-4Rα is expressed naturally in vivo in N-linked glycosylated form. Glycosylated IL-4Rα may also be expressed in recombinant systems, e.g. in HEK-EBNA cells. IL-4Rα may also be expressed in non-glycosylated form in E. coli cells.

### Antibody Molecule

This describes an immunoglobulin whether natural or partly or wholly synthetically produced. The term also covers any polypeptide or protein comprising an antibody antigen-binding site. It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described later. Antibody fragments that comprise an antibody antigen-binding site include, but are not limited to molecules such as Fab, Fab′, Fab′-SH, scFv, Fv, dAb, Fd; and diabodies.

Antibody molecules of the invention may be IgG, e.g. IgG1, IgG4, IgG2 or a glycosyl IgG2.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

As antibodies can be modified in a number of ways, the term “antibody molecule” should be construed as covering any binding member or substance having an antibody antigen-binding site with the required specificity and/or binding to antigen. Thus, this term covers antibody fragments and derivatives, including any polypeptide comprising an antibody antigen-binding site, whether natural or wholly or partially synthetic. Chimeric molecules comprising an antibody antigen-binding site, or equivalent, fused to another polypeptide (e.g. derived from another species or belonging to another antibody class or subclass) are therefore included. Cloning and expression of chimeric antibodies are described in EP-A-0120694 and EP-A-0125023, and a large body of subsequent literature.

Further techniques available in the art of antibody engineering have made it possible to isolate human and humanised antibodies. For example, human hybridomas can be made as described by Kontermann & Dubel (Antibody Engineering, Springer-Verlag New York, LLC; 2001, ISBN: 3540413545). Phage display, another established technique for generating antibodies has been described in detail in many publications such as WO92/01047 (discussed further below) and U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160, U.S. Pat. No. 6,521,404 and Kontermann & Dubel (supra). Transgenic mice in which the mouse antibody genes are inactivated and functionally replaced with human antibody genes while leaving intact other components of the mouse immune system, can be used for isolating human antibodies (Mendez et al. Nature Genet, 15(2): 146-156, 1997).

Synthetic antibody molecules may be created by expression from genes generated by means of oligonucleotides synthesized and assembled within suitable expression vectors, for example as described by Knappik et al. (J. Mol. Biol. 296, 57-86, 2000) or Krebs et al. (Journal of Immunological Methods, 254:67-84, 2001).

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al., Nature 341:544-546, 1989; McCafferty et al. Nature, 348:552-554, 1990; Holt et al. Trends in Biotechnology 21, 484-490, 2003), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. Science, 242, 423-426, 1988; Huston PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; Holliger et al, PNAS USA 90:6444-6448, 1993a). Fv, scFv or diabody molecules may be stabilized by the incorporation of disulphide bridges linking the VH and VL domains (Reiter et al, Nature Biotech, 14:1239-1245, 1996). Minibodies comprising a scFv joined to a CH3 domain may also be made (Hu et al, Cancer Res., 56, 3055-3061, 1996). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Antibody fragments of the invention can be obtained starting from any of the antibody molecules described herein, e.g. antibody molecules comprising VH and/or VL domains or CDRs of any of Antibodies 1 to 42, by methods such as digestion by enzymes, such as pepsin or papain and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc., or by nucleic acid synthesis and expression.

Functional antibody fragments according to the present invention include any functional fragment whose half-life is increased by a chemical modification, especially by PEGylation, or by incorporation in a liposome.

A dAb (domain antibody) is a small monomeric antigen-binding fragment of an antibody, namely the variable region of an antibody heavy or light chain (Holt et al. Trends in Biotechnology 21, 484-490, 2003). VH dAbs occur naturally in camelids (e.g. camel, llama) and may be produced by immunizing a camelid with a target antigen, isolating antigen-specific B cells and directly cloning dAb genes from individual B cells. dAbs are also producible in cell culture. Their small size, good solubility and temperature stability makes them particularly physiologically useful and suitable for selection and affinity maturation. An antibody of the present invention may be a dAb comprising a VH or VL domain substantially as set out herein, or a VH or VL domain comprising a set of CDRs substantially as set out herein.

As used herein, the phrase “substantially as set out” refers to the characteristic(s) of the relevant CDRs of the VH or VL domain of antibodies described herein will be either identical or highly similar to the specified regions of which the sequence is set out herein. As described herein, the phrase “highly similar” with respect to specified region(s) of one or more variable domains, it is contemplated that from 1 to about 12, e.g. from 1 to 8, including 1 to 7, 1 to 6, 1 to 5, 1 to 4, 1 to 3, or 1 or 2, amino acid substitutions may be made in the CDRs of the VH and/or VL domain.

Antibodies of the invention include bispecific antibodies. Bispecific or bifunctional antibodies form a second generation of monoclonal antibodies in which two different variable regions are combined in the same molecule (Holliger, P. & Winter, G. 1999 Cancer and metastasis rev. 18:411-419, 1999). Their use has been demonstrated both in the diagnostic field and in the therapy field from their capacity to recruit new effector functions or to target several molecules on the surface of tumor cells. Where bispecific antibodies are to be used, these may be conventional bispecific antibodies, which can be manufactured in a variety of ways (Holliger et al, PNAS USA 90:6444-6448, 1993), e.g. prepared chemically or from hybrid hybridomas, or may be any of the bispecific antibody fragments mentioned above. These antibodies can be obtained by chemical methods (Glennie et al., 1987 J. Immunol. 139, 2367-2375; Repp et al., J. Hemat. 377-382, 1995) or somatic methods (Staerz U. D. and Bevan M. J. PNAS 83, 1986; et al., Method Enzymol. 121:210-228, 1986) but likewise by genetic engineering techniques which allow the heterodimerization to be forced and thus facilitate the process of purification of the antibody sought (Merchand et al. Nature Biotech, 16:677-681, 1998). Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific antibodies can be constructed as entire IgG, as bispecific F(ab′)2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against IL-4Rα, then a library can be made where the other arm is varied and an antibody of appropriate specificity selected. Bispecific whole antibodies may be made by alternative engineering methods as described in Ridgeway et al, (Protein Eng., 9:616-621, 1996).

Various methods are available in the art for obtaining antibodies against IL-4Rα. The antibodies may be monoclonal antibodies, especially of human, murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein (Nature, 256:495-497, 1975).

Monoclonal antibodies can be obtained, for example, from an animal cell immunized against IL-4Rα, or one of their fragments containing the epitope recognized by said monoclonal antibodies. The IL-4Rα, or one of its fragments, can especially be produced according to the usual working methods, by genetic recombination starting with a nucleic acid sequence contained in the cDNA sequence coding for IL-4Rα or fragment thereof, by peptide synthesis starting from a sequence of amino acids comprised in the peptide sequence of the IL-4Rα and/or fragment thereof. The monoclonal antibodies can, for example, be purified on an affinity column on which IL-4Rα or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the LPS, in itself, followed or not followed by exclusion chromatography on Sepharose gel in order to eliminate the potential aggregates due to the presence of dimers or of other multimers. In one embodiment, the whole of these techniques can be used simultaneously or successively.

An antigen binding site may be provided by means of arrangement of CDRs on non-antibody protein scaffolds such as fibronectin or cytochrome B etc. (Haan & Maggos, BioCentury, 12(5):A1-A6, 2004; Koide, Journal of Molecular Biology, 284:1141-1151, 1998; Nygren et al., Current Opinion in Structural Biology, 7:463-469, 1997), or by randomising or mutating amino acid residues of a loop within a protein scaffold to confer binding specificity for a desired target. Scaffolds for engineering novel binding sites in proteins have been reviewed in detail by Nygren et al. (supra). Protein scaffolds for antibody mimics are disclosed in WO/0034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs or an HCDR and/or LCDR3, may be provided by any domain member of the immunoglobulin gene super family. The scaffold may be a human or non-human protein.

In addition to antibody sequences and/or an antigen-binding site, a antibody according to the present invention may comprise other amino acids, e.g. forming a peptide or polypeptide, such as a folded domain, or to impart to the molecule another functional characteristic in addition to ability to bind antigen. Antibodies of the invention may carry a detectable label, or may be conjugated to a toxin or a targeting moiety or enzyme (e.g. via a peptidyl bond or linker). For example, an antibody may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

The structure for carrying a CDR, e.g. CDR3, or a set of CDRs of the invention will generally be an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to Kabat (Sequences of Proteins of Immunological Interest, 4^{th} Edition. US Department of Health and Human Devices, 1987), and updates thereof, such as the 5^{th} Edition (Sequences of Proteins of Immunological Interest, 5th Edition. US Department of Health and Human Services, Public Service, NIH, Washington, 1991).

Unless indicated otherwise, the locations of particular residues, as well as CDR and framework regions, referred to herein uses the Kabat numbering system.

By CDR region or CDR, it is intended to indicate the hypervariable regions of the heavy and light chains of the immunoglobulin as defined by Kabat et al., (supra). An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

Among the six short CDR sequences, the third CDR of the heavy chain (HCDR3) has greater size variability (greater diversity essentially due to the mechanisms of arrangement of the genes which give rise to it). It can be as short as 2 amino acids although the longest size known is 26. Functionally, HCDR3 plays a role in part in the determination of the specificity of the antibody (Segal et al. PNAS, 71:4298-4302, 1974; Amit et al., Science, 233:747-753, 1986; Chothia et al. J. Mol. Biol., 196:901-917, 1987; Chothia et al. Nature, 342:877-883, 1989; et al. J. Immunol., 144:1965-1968, 1990; Sharon et al. PNAS, 87:4814-4817, 1990(a); Sharon et al. J. Immunol., 144:4863-4869, 1990; Kabat et al., et al., J. Immunol., 147:1709-1719, 1991b).
HCDR1 may be 5 amino acids long, consisting of Kabat residues 31-35.
HCDR2 may be 17 amino acids long, consisting of Kabat residues 50-65.
HCDR3 may be 7 amino acids long, consisting of Kabat residues 95-102.
LCDR1 may be 13 amino acids long, consisting of Kabat residues 24-34.
LCDR2 may be 7 amino acids long, consisting of Kabat residues 50-56.
LCDR3 may be 12 amino acids long, consisting of Kabat residues 89-97.

### Antigen-Binding Site

This describes the part of a molecule that binds to and is complementary to all or part of the target antigen. In an antibody molecule it is referred to as the antibody antigen-binding site, and comprises the part of the antibody that binds to and is complementary to all or part of the target antigen. Where an antigen is large, an antibody may only bind to a particular part of the antigen, which part is termed an epitope. An antibody antigen-binding site may be provided by one or more antibody variable domains. An antibody antigen-binding site may comprise an antibody light chain variable region (VL) and an antibody heavy chain variable region (VH).

### Isolation

This refers to the state in which antibodies of the invention, or nucleic acid encoding such antibodies, will generally be in accordance with the present invention. Thus, antibodies, including VH and/or VL domains, and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes of origin other than the sequence encoding a polypeptide with the required function. Isolated members and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practiced in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Antibodies may be glycosylated, either naturally or by systems of heterologous eukaryotic cells (e.g. CHO or NS0 (ECACC 85110503)) cells, or they may be (for example if produced by expression in a prokaryotic cell) unglycosylated.

Heterogeneous preparations comprising anti-IL-4Rα antibody molecules also form part of the invention. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

As noted above, an antibody in accordance with the present invention modulates and may neutralize a biological activity of IL-4Rα. As described herein, IL-4Rα-antibodies of the present invention may be optimized for neutralizing potency. Generally, potency optimization involves mutating the sequence of a selected antibody (normally the variable domain sequence of an antibody) to generate a library of antibodies, which are then assayed for potency and the more potent antibodies are selected. Thus selected “potency-optimized” antibodies tend to have a higher potency than the antibodies from which the library was generated. Nevertheless, high potency antibodies may also be obtained without optimization, for example a high potency antibody may be obtained directly from an initial screen e.g. a biochemical neutralization assay. A “potency optimized” antibody refers to an antibody with an optimized potency of binding or neutralization of a particular activity or downstream function of IL-4Rα. Assays and potencies are described in more detail elsewhere herein. The present invention provides both potency-optimized and non-optimized antibodies, as well as methods for potency optimization from a selected antibody. The present invention thus allows the skilled person to generate compositions having antibodies with high potency.

Although potency optimization may be used to generate higher potency antibodies from a given binding member, it is also noted that high potency antibodies may be obtained even without potency optimization.

An antibody VH domain with the amino acid sequence of an antibody VH domain of a said selected binding member may be provided in isolated form, as may an antibody comprising such a VH domain.

Ability to bind IL-4Rα and/or ability to compete with e.g. a parent antibody molecule (e.g. Antibody 1) or an optimized antibody molecule, Antibodies 2 to 42 (e.g. in scFv format and/or IgG format, e.g. IgG 1, IgG2 or IgG4) for binding to IL-4Rα, may be further tested. Ability to neutralize IL-4Rα may be tested, as discussed further elsewhere herein.

An antibody according to the present invention may bind IL-4Rα with the affinity of one of Antibodies 1 to 42, e.g. in scFv or IgG 1 or IgG2 or IgG4 format, or with an affinity that is better.

An antibody according to the present invention may neutralize a biological activity of IL-4Rα with the potency of one of Antibodies 1 to 42 e.g. in scFv or IgG 1 or IgG2 or IgG4 format, or with a potency that is better.

Binding affinity and neutralization potency of different antibodies can be compared under appropriate conditions.

Variants of antibody molecules disclosed herein may be produced and used in the present invention. Following the lead of computational chemistry in applying multivariate data analysis techniques to the structure/property-activity relationships (Wold et al Multivariate data analysis in chemistry. Chemometrics—Mathematics and Statistics in Chemistry (Ed.: B. Kowalski), D. Reidel Publishing Company, Dordrecht, Holland, 1984 (ISBN 90-277-1846-6)) quantitative activity-property relationships of antibodies can be derived using well-known mathematical techniques such as statistical regression, pattern recognition and classification (Norman et al. Applied Regression Analysis. Wiley-Interscience; 3^{rd} edition (April 1998) ISBN: 0471170828; Kandel, Abraham & Backer, Computer-Assisted Reasoning in Cluster Analysis. Prentice Hall PTR, (May 11, 1995), ISBN: 0133418847; Principles of Multivariate Analysis: A User's Perspective (Oxford Statistical Science Series, No 22 (Paper)). Oxford University Press; (December 2000), ISBN: 0198507089; Witten & Frank Data Mining: Practical Machine Learning Tools and Techniques with Java Implementations. Morgan Kaufmann; (Oct. 11, 1999), ISBN: 1558605525; Denison DGT. (Editor), Holmes, C. C. et al. Bayesian Methods for Nonlinear Classification and Regression (Wiley Series in Probability and Statistics). John Wiley & Sons; (July 2002), ISBN: 0471490369; Ghose, A K. & Viswanadhan, V N. Combinatorial Library Design and Evaluation Principles, Software, Tools, and Applications in Drug Discovery. ISBN: 0-8247-0487-8). The properties of antibodies can be derived from empirical and theoretical models (for example, analysis of likely contact residues or calculated physicochemical property) of antibody sequence, functional and three-dimensional structures and these properties can be considered singly and in combination.

The techniques required to make substitutions within amino acid sequences of CDRs, antibody VH or VL domains and antibodies generally are available in the art. Variant sequences may be made, with substitutions that may or may not be predicted to have a minimal or beneficial effect on activity, and tested for ability to bind and/or neutralize IL-4Rα and/or for any other desired property.

Variable domain amino acid sequence variants of any of the VH and VL domains whose sequences are specifically disclosed herein may be employed in accordance with the present invention, as discussed. Particular variants may include one or more amino acid sequence alterations (addition, deletion, substitution and/or insertion of an amino acid residue), may be less than about 20 alterations, less than about 15 alterations, less than about 12 alterations, less than about 10 alterations, or less than about 6 alterations, maybe 5, 4, 3, 2 or 1. Alterations may be made in one or more framework regions and/or one or more CDRs. The alterations normally do not result in loss of function, so an antibody comprising a thus-altered amino acid sequence may retain an ability to bind and/or neutralize IL-4Rα. For example, it may retain the same quantitative binding and/or neutralizing ability as an antibody in which the alteration is not made, e.g. as measured in an assay described herein. The binding member comprising a thus-altered amino acid sequence may have an improved ability to bind and/or neutralize IL-4Rα. Indeed, Antibodies 21 to 42, generated from random mutagenesis of Antibody 20, exhibits substitutions relative to Antibody 20, mostly within the various framework regions and each of these still bind and/or neutralizes IL-4Rα, indeed some show improved ability to bind and/or neutralize IL-4Rα.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Example numbers and locations of alterations in sequences of the invention are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, Biochemistry, 2nd Edition, (Wiley) 1995). Those amino acid residues that are derivatized at their N-alpha position will only be located at the N-terminus of an amino-acid sequence. Normally in the present invention an amino acid is an L-amino acid, but in some embodiments it may be a D-amino acid. Alteration may therefore comprise modifying an L-amino acid into, or replacing it with, a D-amino acid. Methylated, acetylated and/or phosphorylated forms of amino acids are also known, and amino acids in the present invention may be subject to such modification.

Amino acid sequences in antibody domains and antibodies of the invention may comprise non-natural or non-standard amino acids described above. In some embodiments non-standard amino acids (e.g. D-amino acids) may be incorporated into an amino acid sequence during synthesis, while in other embodiments the non-standard amino acids may be introduced by modification or replacement of the “original” standard amino acids after synthesis of the amino acid sequence.

Use of non-standard and/or non-naturally occurring amino acids increases structural and functional diversity, and can thus increase the potential for achieving desired IL-4Rα-binding and neutralizing properties in an antibody of the invention. Additionally, D-amino acids and analogues have been shown to have better pharmacokinetic profiles compared with standard L-amino acids, owing to in vivo degradation of polypeptides having L-amino acids after administration to an animal e.g. a human.

Novel VH or VL regions carrying CDR-derived sequences of the invention may be generated using random mutagenesis of one or more selected VH and/or VL genes to generate mutations within the entire variable domain. Such a technique is described by Gram et al. (Proc. Natl. Acad. Sci., USA, 89:3576-3580, 1992), who used error-prone PCR. In some embodiments one or two amino acid substitutions are made within an entire variable domain or set of CDRs. Another method that may be used is to direct mutagenesis to CDR regions of VH or VL genes. Such techniques are disclosed by Barbas et al. (Proc. Natl. Acad. Sci., 91:3809-3813, 1994) and Schier et al. (J. Mol. Biol. 263:551-567, 1996).

All the above-described techniques are known as such in the art and the skilled person will be able to use such techniques to provide antibodies of the invention using routine methodology in the art.

A further aspect of the invention provides a method for obtaining an antibody antigen-binding site for IL-4Rα, the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify an antibody or an antibody antigen-binding site for IL-4Rα and optionally with one or more functional properties, e.g. ability to neutralize IL-4Rα activity. Said VL domain may have an amino acid sequence which is substantially as set out herein. An analogous method may be employed in which one or more sequence variants of a VL domain disclosed herein are combined with one or more VH domains.

As noted above, a CDR amino acid sequence substantially as set out herein may be carried as a CDR in a human antibody variable domain or a substantial portion thereof. The HCDR3 sequences substantially as set out herein represent embodiments of the present invention and for example each of these may be carried as a HCDR3 in a human heavy chain variable domain or a substantial portion thereof.

Variable domains employed in the invention may be obtained or derived from any germ-line or rearranged human variable domain, or may be a synthetic variable domain based on consensus or actual sequences of known human variable domains. A variable domain can be derived from a non-human antibody. A CDR sequence of the invention (e.g. CDR3) may be introduced into a repertoire of variable domains lacking a CDR (e.g. CDR3), using recombinant DNA technology. For example, Marks et al. (Bio/Technology, 10:779-783, 1992) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide antibodies of the invention. The repertoire may then be displayed in a suitable host system such as the phage display system of WO92/01047, which is herein incorporated by reference in its entirety, or any of a subsequent large body of literature, including Kay, Winter & McCafferty (Phage Display of Peptides and Proteins: A Laboratory Manual, San Diego: Academic Press, 1996), so that suitable antibodies may be selected. A repertoire may consist of from anything from 10⁴ individual members upwards, for example at least 10⁵, at least 10⁶, at least 10⁷, at least 10⁸, at least 10⁹ or at least 10¹⁰ members. Other suitable host systems include, but are not limited to, yeast display, bacterial display, T7 display, viral display, cell display, ribosome display and covalent display.

Analogous shuffling or combinatorial techniques are also disclosed by Stemmer (Nature, 370:389-391, 1994), who describes the technique in relation to a β-lactamase gene but observes that the approach may be used for the generation of antibodies.

Again, an analogous method may be employed in which a VL CDR3 of the invention is combined with a repertoire of nucleic acids encoding a VL domain that either include a CDR3 to be replaced or lack a CDR3 encoding region.

Similarly, other VH and VL domains, sets of CDRs and sets of HCDRs and/or sets of LCDRs disclosed herein may be employed.

Similarly, one or more, or all three CDRs may be grafted into a repertoire of VH or VL domains that are then screened for an antibody or antibodies for IL-4Rα.

Alternatively, nucleic acid encoding the VH and/or VL domains of any of an antibody of the present invention, e.g. Antibodies 1-42, can be subjected to mutagenesis (e.g. targeted or random) to generate one or more mutant nucleic acids. Antibodies encoded by these sequences can then be generated.

In one embodiment, one or more of Antibodies 1 to 42 HCDR1, HCDR2 and HCDR3, or an Antibody 1 to 42 set of HCDRs, may be employed, and/or one or more of Antibodies 1 to 42 LCDR1, LCDR2 and LCDR3 or an Antibody 1 to 42 set of LCDRs may be employed.

In particular embodiments the donor nucleic acid is produced by targeted or random mutagenesis of the VH or VL domains or any CDR region therein.

In another embodiment, the product VH or VL domain is attached to an antibody constant region.

In another embodiment the product VH or VL domain and a companion VL or VH domain respectively, is comprised in an IgG, scFV or Fab antibody molecule.

In another embodiment the recovered binding member or antibody molecule is tested for ability to neutralize IL-4Rα.

In some embodiments, a substantial portion of an immunoglobulin variable domain will comprise at least the three CDR regions, together with their intervening framework regions. The portion may also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of antibodies of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including antibody constant regions, other variable domains (for example in the production of diabodies) or detectable/functional labels as discussed in more detail elsewhere herein.

Although in some aspects of the invention, antibodies comprise a pair of VH and VL domains, single binding domains based on either VH or VL domain sequences form further aspects of the invention. It is known that single immunoglobulin domains, especially VH domains, are capable of binding target antigens in a specific manner. For example, see the discussion of dAbs above.

In the case of either of the single binding domains, these domains may be used to screen for complementary domains capable of forming a two-domain binding member able to bind IL-4Rα. This may be achieved by phage display screening methods using the so-called hierarchical dual combinatorial approach as disclosed in WO92/01047, herein incorporated by reference in its entirety, in which an individual colony containing either an H or L chain clone is used to infect a complete library of clones encoding the other chain (L or H) and the resulting two-chain binding member is selected in accordance with phage display techniques such as those described in that reference. This technique is also disclosed in Marks et al. (Bio/Technology, 10:779-783, 1992).

Antibodies of the present invention may further comprise antibody constant regions or parts thereof, e.g. human antibody constant regions or parts thereof. For example, a VL domain may be attached at its C-terminal end to antibody light chain constant domains including human Cκ or Cλ chains, e.g. Cλ chains. Similarly, an antibody based on a VH domain may be attached at its C-terminal end to all or part (e.g. a CH1 domain) of an immunoglobulin heavy chain derived from any antibody isotype, e.g. IgG, IgA, IgD, IgY, IgE and IgM and any of the isotype sub-classes (e.g., IgG1, IgG2, IgG3, IgG4, IgA1 and IgA2; particularly IgG1 and IgG4). IgG1 is advantageous, due to its effector function and ease of manufacture. Any synthetic or other constant region variant that has these properties and stabilizes variable regions is also useful in embodiments of the present invention.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate ADCC in humans. Human light chain constant regions may be classified into two major classes, kappa and lambda.

### Antibody Format

The present invention also includes antibodies of the invention, and in particular the antibodies of the invention, that have modified IgG constant domains. Antibodies of the human IgG class, which have functional characteristics such a long half-life in serum and the ability to mediate various effector functions are used in certain embodiments of the invention (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. A large number of studies have so far been conducted for ADCC and CDC as effector functions of the IgG class antibody, and it has been reported that among antibodies of the human IgG class, the IgG1 subclass has the highest ADCC activity and CDC activity in humans (Chemical Immunology, 65, 88 (1997)).

Thus according to a further aspect of the invention there is provided antibodies, in particular antibodies, which have been modified so as to change, i.e. increase, decrease or eliminate, the biological effector function of the antibodies, for example antibodies with modified Fc regions. In some embodiments, the antibodies as disclosed herein can be modified to enhance their capability of fixing complement and participating in complement-dependent cytotoxicity (CDC). In other embodiments, the antibodies can be modified to enhance their capability of activating effector cells and participating in antibody-dependent cytotoxicity (ADCC). In yet other embodiments, the antibodies as disclosed herein can be modified both to enhance their capability of activating effector cells and participating in antibody-dependent cytotoxicity (ADCC) and to enhance their capability of fixing complement and participating in complement-dependent cytotoxicity (CDC).

In some embodiments, the antibodies as disclosed herein can be modified to reduce their capability of fixing complement and participating in complement-dependent cytotoxicity (CDC). In other embodiments, the antibodies can be modified to reduce their capability of activating effector cells and participating in antibody-dependent cytotoxicity (ADCC). In yet other embodiments, the antibodies as disclosed herein can be modified both to reduce their capability of activating effector cells and participating in antibody-dependent cytotoxicity (ADCC) and to reduce their capability of fixing complement and participating in complement-dependent cytotoxicity (CDC).

In one embodiment, an antibody with an Fc variant region has enhanced ADCC activity relative to a comparable molecule. In a specific embodiment, an antibody with an Fc variant region has ADCC activity that is at least 2 fold, or at least 3 fold, or at least 5 fold or at least 10 fold or at least 50 fold or at least 100 fold greater than that of a comparable molecule. In another specific embodiment, an antibody with an Fc variant region has enhanced binding to the Fc receptor FcγRIIIA and has enhanced ADCC activity relative to a comparable molecule. In other embodiments, the binding member with an Fc variant region has both enhanced ADCC activity and enhanced serum half-life relative to a comparable molecule.

In one embodiment, an antibody with an Fc variant region has reduced ADCC activity relative to a comparable molecule. In a specific embodiment, an antibody with an Fc variant region has ADCC activity that is at least 2 fold, or at least 3 fold, or at least 5 fold or at least 10 fold or at least 50 fold or at least 100 fold lower than that of a comparable molecule. In another specific embodiment, the binding member with an Fc variant region has reduced binding to the Fc receptor FcγRIIIA and has reduced ADCC activity relative to a comparable molecule. In other embodiments, the binding member with an Fc variant region has both reduced ADCC activity and enhanced serum half-life relative to a comparable molecule.

In one embodiment, the binding member with an Fc variant region has enhanced CDC activity relative to a comparable molecule. In a specific embodiment the binding member with an Fc variant region has CDC activity that is at least 2 fold, or at least 3 fold, or at least 5 fold or at least 10 fold or at least 50 fold or at least 100 fold greater than that of a comparable molecule. In other embodiments, the binding member with an Fc variant region has both enhanced CDC activity and enhanced serum half-life relative to a comparable molecule.

In one embodiment, the binding member with an Fc variant region has reduced binding to one or more Fc ligand relative to a comparable molecule. In another embodiment, the binding member with an Fc variant region has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold lower than that of a comparable molecule. In a specific embodiment, the binding member with an Fc variant region has reduced binding to an Fc receptor. In another specific embodiment, the binding member with an Fc variant region has reduced binding to the Fc receptor FcγRIIIA In a further specific embodiment, an binding member with an Fc variant region described herein has an affinity for the Fc receptor FcγRIIIA that is at least about 5 fold lower than that of a comparable molecule, wherein said Fc variant has an affinity for the Fc receptor FcγRIIB that is within about 2 fold of that of a comparable molecule. In still another specific embodiment, the binding member with an Fc variant region has reduced binding to the Fc receptor FcRn. In yet another specific embodiment, the binding member with an Fc variant region has reduced binding to C1q relative to a comparable molecule.

In one embodiment, the binding member with the Fc variant region has enhanced binding to one or more Fc ligand(s) relative to a comparable molecule. In another embodiment, the binding member with the Fc variant region has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, the binding member with the Fc variant region has enhanced binding to an Fc receptor. In another specific embodiment, the binding member with the Fc variant region has enhanced binding to the Fc receptor FcγRIIIA In a further specific embodiment, the binding member with the Fc variant region has enhanced biding to the Fc receptor FcγRIIB In still another specific embodiment, the binding member with the Fc variant region has enhanced binding to the Fc receptor FcRn. In yet another specific embodiment, the binding member with the Fc variant region has enhanced binding to C1q relative to a comparable molecule.

In one embodiment, an anti-IL-4Rα antibody of the invention comprises a variant Fc domain wherein said variant Fc domain has enhanced binding affinity to Fc gamma receptor IIB relative to a comparable non-variant Fc domain. In a further embodiment, an anti-IL-4Rα antibody of the invention comprises a variant Fc domain wherein said variant Fc domain has an affinity for Fc gamma receptor IIB that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

In one embodiment, the present invention provides an antibody with an Fc variant region or formulations comprising these, wherein the Fc region comprises a non-native amino acid residue at one or more positions selected from the group consisting of 228, 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non-native amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

By “non-native amino acid residue”, we mean an amino acid residue that is not present at the recited position in the naturally occurring protein. Typically, this will mean that the or a native/natural amino acid residue has been substituted for one or more other residues, which may comprise one of the other 20 naturally-occurring (common) amino acids or a non-classical amino acids or a chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In a specific embodiment, the present invention provides an antibody with an variant Fc region or a formulation comprising such binding member with an variant Fc region, wherein the Fc region comprises at least one non-native amino acid residue selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 2981, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 3281, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non-native amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An variant Fc protein may be an antibody, Fc fusion, or any protein or protein domain that comprises an Fc region including, but not limited to, proteins comprising variant Fc regions, which are non naturally occurring variants of an Fc.

The present invention encompasses antibodies with variant Fc regions, which have altered binding properties for an Fc ligand (e.g., an Fc receptor, C1q) relative to a comparable molecule (e.g., a protein having the same amino acid sequence except having a wild type Fc region). Examples of binding properties include but are not limited to, binding specificity, equilibrium dissociation constant (K_{D}), dissociation and association rates (k_{off} and kₒₙ respectively), binding affinity and/or avidity. It is generally understood that a binding molecule (e.g., a variant Fc protein such as an antibody) with a low K_{D} may be preferable to a binding molecule with a high K_{D}. However, in some instances the value of the kₒₙ or k_{off} may be more relevant than the value of the K_{D}. One skilled in the art can determine which kinetic parameter is most important for a given antibody application.

The affinities and binding properties of an Fc domain for its ligand may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. The serum half-life of proteins comprising Fc regions may be increased by increasing the binding affinity of the Fc region for FcRn. In one embodiment, the Fc variant protein has enhanced serum half-life relative to comparable molecule.

### Conjugation and Half Life

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

In certain embodiments, the half-life of an anti-IL-4Rα antibody or compositions and methods of the invention is at least about 4 to 7 days. In certain embodiments, the mean half-life of an anti-IL-4Rα antibody of compositions and methods of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of an anti-IL-4Rα antibody of compositions and methods of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of an anti-IL-4Rα antibody of compositions and methods of the invention can be up to about 50 days. In certain embodiments, the half-lives of antibodies of compositions and methods of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375, U.S. Pat. No. 7,083,784; and International Publication Nos. WO 98/23289 and WO 97/3461.

The serum circulation of anti-IL-4Rα antibodies in vivo may also be prolonged by attaching inert polymer molecules such as high molecular weight polyethyleneglycol (PEG) to the antibodies with or without a multifunctional linker either through site-specific conjugation of the PEG to the N- or C-terminus of the antibodies or via epsilon-amino groups present on lysyl residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Further, the antibodies of compositions and methods of the invention can be conjugated to albumin in order to make the antibody more stable in vivo or have a longer half-life in vivo. The techniques are well known in the art, see, e.g., International Publication Nos. WO 93/15199, WO 93/15200, and WO 01/77137; and European Patent No. EP 413, 622, all of which are incorporated herein by reference.

In certain embodiments, the half-life of an antibody as disclosed herein and of compositions of the invention is at least about 4 to 7 days. In certain embodiments, the mean half-life of an antibody as disclosed herein and of compositions of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of an antibody as disclosed herein and of compositions of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of an antibody as disclosed herein and of compositions of the invention can be up to about 50 days. In certain embodiments, the half-lives of antibodies and of compositions of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; U.S. Pat. No. 7,083,784; and International Publication Nos. WO 1998/23289 and WO 1997/34361.

### Mutations and Modifications

In another embodiment, the present invention provides an antibody with a variant Fc region, or a formulation comprising these, wherein the Fc region comprises at least one non-native modification at one or more positions selected from the group consisting of 239, 330 and 332, as numbered by the EU index as set forth in Kabat. In a specific embodiment, the present invention provides an Fc variant, wherein the Fc region comprises at least one non-native amino acid selected from the group consisting of 239D, 330L and 332E, as numbered by the EU index as set forth in Kabat. Optionally, the Fc region may further comprise additional non-native amino acid at one or more positions selected from the group consisting of 252, 254, and 256, as numbered by the EU index as set forth in Kabat. In a specific embodiment, the present invention provides an Fc variant, wherein the Fc region comprises at least one non-native amino acid selected from the group consisting of 239D, 330L and 332E, as numbered by the EU index as set forth in Kabat and at least one normative amino acid at one or more positions selected from the group consisting of 252Y, 254T and 256E, as numbered by the EU index as set forth in Kabat.

In another embodiment, the present invention provides an antibody with a variant Fc region, or a formulation comprising these, wherein the Fc region comprises at least one non-native amino acid at one or more positions selected from the group consisting of 234, 235 and 331, as numbered by the EU index as set forth in Kabat. In a specific embodiment, the present invention provides an Fc variant, wherein the Fc region comprises at least one non-native amino acid selected from the group consisting of 234F, 235F, 235Y, and 331, Sas numbered by the EU index as set forth in Kabat. In a further specific embodiment, an Fc variant of the invention comprises the 234F, 235F, and 331S amino acid residues, as numbered by the EU index as set forth in Kabat. In another specific embodiment, an Fc variant of the invention comprises the 234F, 235Y, and 331S amino acid residues, as numbered by the EU index as set forth in Kabat. Optionally, the Fc region may further comprise additional non-native amino acid residues at one or more positions selected from the group consisting of 252, 254, and 256, as numbered by the EU index as set forth in Kabat. In a specific embodiment, the present invention provides an Fc variant, wherein the Fc region comprises at least one non-native amino acid selected from the group consisting of 234F, 235F, 235Y, and 331S, as numbered by the EU index as set forth in Kabat; and at least one non-native amino acid at one or more positions selected from the group consisting of 252Y, 254T and 256E, as numbered by the EU index as set forth in Kabat.

In a particular embodiment, the invention provides an antibody of the present invention with a variant Fc region, wherein the variant comprises a tyrosine (Y) residue at position 252, a threonine (T) residue at position 254 and a glutamic acid (E) residue at position 256, as numbered by the EU index as set forth in Kabat.

The M252Y, S254T and T256E mutations, as numbered by the EU index as set forth in Kabat, hereinafter referred to as YTE mutations, have been reported to increase serum half-life of a particular IgG1 antibody molecule (Dall'Acqua et al. J. Biol. Chem. 281(33):23514-23524, 2006).

In a further embodiment, the invention provides an antibody of the present invention with a variant Fc region, wherein the variant comprises a tyrosine (Y) residue at position 252, a threonine (T) residue at position 254, a glutamic acid (E) residue at position 256 and a proline (P) residue at position 241, as numbered by the EU index as set forth in Kabat.

The serine228proline mutation (S228P), as numbered by the EU index as set forth in Kabat, hereinafter referred to as the P mutation, has been reported to increase the stability of a particular IgG4 molecule (Lu et al., J Pharmaceutical Sciences 97(2):960-969, 2008). Note: In Lu et al. it is referred to as position 241 because therein they use the Kabat numbering system, not the “EU index” as set forth in Kabat.

This P mutation may be combined with L235E to further knock out ADCC. This combination of mutations is hereinafter referred to as the double mutation (DM).

In a particular embodiment, the invention provides an antibody of the present invention with a variant Fc region, wherein the variant comprises a phenylalanine (F) residue at position 234, a phenylalanine (F) residue or a glutamic acid (E) residue at position 235 and a serine (S) residue at position 331, as numbered by the EU index as set forth in Kabat. Such a mutation combinations are hereinafter referred to as the triple mutant (TM).

According to a further embodiment, the invention provides an antibody of the present invention in IgG1 format with the YTE mutations in the Fc region.

According to a further embodiment, the invention provides an antibody of the present invention in IgG1 format with the TM mutations in the Fc region.

According to a further embodiment, the invention provides an antibody of the present invention in IgG1 format with the YTE mutations and the TM mutations in the Fc region.

According to embodiment, the invention provides an antibody of the present invention in IgG4 format with the YTE and P mutations in the Fc region.

According to embodiment, the invention provides an antibody of the present invention in IgG4 format with the YTE and DM mutations in the Fc region.

According to particular embodiments of the inventions there is provided an antibody of the present invention in a format selected from: IgG1 YTE, IgG1 TM, IgG1 TM+YTE, IgG4 P, IgG4 DM, IgG4 YTE, IgG4 P+YTE and IgG4 DM+YTE.

In terms of the nomenclature used, it will be appreciated that DM+YTE means that the constant domain Fc region possesses both the double mutations (S228P and L235E) and the YTE mutations (M252Y, S254T and T256E).

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492, 1985), PCR mutagenesis (Higuchi, in “PCR Protocols: A Guide to Methods and Applications”, Academic Press, San Diego, pp. 177-183, 1990), and cassette mutagenesis (Wells et al., Gene 34:315-323, 1985). Preferably, site-directed mutagenesis is performed by the overlap-extension PCR method (Higuchi, in “PCR Technology: Principles and Applications for DNA Amplification”, Stockton Press, New York, pp. 61-70, 1989). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351; WO 06/23403).

In some embodiments of the invention, the glycosylation patterns of the antibodies provided herein are modified to enhance ADCC and CDC effector function. (See Shields R L et al., (JBC. 277:26733-26740, 2002; Shinkawa T et al., JBC. 278:3466-3473, 2003; and Okazaki A et al., J. Mol. Biol., 336:1239, 2004). In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyl transferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, Nat. Biotechnol 17:176-180, 1999; Davies et al., Biotechnol Bioeng 74:288-294, 2007; Shields et al, J Biol Chem 277:26733-26740, 2002; Shinkawa et al., J Biol Chem 278:3466-3473, 2003) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (Glycart Biotechnology AG, Zurich, Switzerland). See, e.g., WO 00/061739; EA01229125; US 20030115614; Okazaki et al., JMB. 336:1239-49, 2004.

### Labeling

Antibodies of the antibody formulation of the invention may be labeled with a detectable or functional label. A label can be any molecule that produces or can be induced to produce a signal, including but not limited to fluorescers, radiolabels, enzymes, chemiluminescers or photosensitizers. Thus, binding may be detected and/or measured by detecting fluorescence or luminescence, radioactivity, enzyme activity or light absorbance.

Suitable labels include, by way of illustration and not limitation, enzymes such as alkaline phosphatase, glucose-6-phosphate dehydrogenase (“G6PDH”) and horseradish peroxidase; dyes; fluorescers, such as fluorescein, rhodamine compounds, phycoerythrin, phycocyanin, allophycocyanin, o-phthaldehyde, fluorescamine, fluorophores such as lanthanide cryptates and chelates (Perkin Elmer and C is Biointernational); chemiluminescers such as isoluminol; sensitizers; coenzymes; enzyme substrates; radiolabels including but not limited to ¹²⁵I, ¹³¹I, ³⁵S, ³²P, ¹⁴C, ³H, ⁵⁷Co, ⁹⁹Tc and ⁷⁵Se and other radiolabels mentioned herein; particles such as latex or carbon particles; metal sol; crystallite; liposomes; cells, etc., which may be further labeled with a dye, catalyst or other detectable group. Suitable enzymes and coenzymes are disclosed in U.S. Pat. No. 4,275,149 and U.S. Pat. No. 4,318,980, each of which are herein incorporated by reference in their entireties. Suitable fluorescers and chemiluminescers are also disclosed in U.S. Pat. No. 4,275,149, which is incorporated herein by reference in its entirety. Labels further include chemical moieties such as biotin that may be detected via binding to a specific cognate detectable moiety, e.g. labeled avidin or streptavidin. Detectable labels may be attached to antibodies of the invention using conventional chemistry known in the art.

There are numerous methods by which the label can produce a signal detectable by external means, for example, by visual examination, electromagnetic radiation, heat, and chemical reagents. The label can also be bound to another binding member that binds the antibody of the invention, or to a support.

The label can directly produce a signal, and therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. This second wavelength emission may also transfer energy to a labeled acceptor molecule, and the resultant energy dissipated from the acceptor molecule by emission of light for example fluorescence resonance energy transfer (FRET). Other labels that directly produce a signal include radioactive isotopes and dyes.

Alternately, the label may need other components to produce a signal, and the signal producing system would then include all the components required to produce a measurable signal, which may include substrates, coenzymes, enhancers, additional enzymes, substances that react with enzymic products, catalysts, activators, cofactors, inhibitors, scavengers, metal ions, and a specific binding substance required for binding of signal generating substances. A detailed discussion of suitable signal producing systems can be found in U.S. Pat. No. 5,185,243, which is herein incorporated herein by reference in its entirety.

The binding member, antibody, or one of its functional fragments, can be present in the form of an immunoconjugate so as to obtain a detectable and/or quantifiable signal. The immunoconjugates can be conjugated, for example, with enzymes such as peroxidase, alkaline phosphatase, alpha-D-galactosidase, glucose oxidase, glucose amylase, carbonic anhydrase, acetylcholinesterase, lysozyme, malate dehydrogenase or glucose 6-phosphate dehydrogenase or by a molecule such as biotin, digoxygenin or 5-bromodeoxyuridine. Fluorescent labels can be likewise conjugated to the immunoconjugates or to their functional fragments according to the invention and especially include fluorescein and its derivatives, fluorochrome, rhodamine and its derivatives, GFP (GFP for “Green Fluorescent Protein”), dansyl, umbelliferone, Lanthanide chelates or cryptates eg. Europium etc.

The immunoconjugates or their functional fragments can be prepared by methods known to the person skilled in the art. They can be coupled to the enzymes or to the fluorescent labels directly or by the intermediary of a spacer group or of a linking group such as a polyaldehyde, like glutaraldehyde, ethylenediaminetetraacetic acid (EDTA), diethylene-triaminepentaacetic acid (DPTA), or in the presence of coupling agents such as those mentioned above for the therapeutic conjugates. The conjugates containing labels of fluorescein type can be prepared by reaction with an isothiocyanate. Other immunoconjugates can likewise include chemoluminescent labels such as luminol and the dioxetanes, bio-luminescent labels such as luciferase and luciferin, or else radioactive labels such as iodine123, iodine125, iodine126, iodine131, iodine133, bromine77, technetium99m, indium111, indium 113m, gallium67, gallium 68, sulphur35, phosphorus32, carbon14, tritium (hydrogen3), cobalt57, selenium75, ruthenium95, ruthenium97, ruthenium103, ruthenium105, mercury107, mercury203, rhenium99m, rhenium 101, rhenium105, scandium47, tellurium121 m, tellurium122m, tellurium125m, thulium165, thulium167, thulium168, fluorine8, yttrium 199. The methods known to the person skilled in the art existing for coupling the therapeutic radioisotopes to the antibodies either directly or via a chelating agent such as EDTA, DTPA mentioned above can be used for the radioelements which can be used in diagnosis. It is likewise possible to mention labelling with Na[I 125] by the chloramine T method (Hunter and Greenwood, Nature, 194:495, 1962) or else with technetium99m by the technique of Crockford et al., (U.S. Pat. No. 4,424,200, herein incorporated by reference in its entirety) or attached via DTPA as described by Hnatowich (U.S. Pat. No. 4,479,930, herein incorporated by reference in its entirety). Further immunoconjugates can include a toxin moiety such as for example a toxin moiety selected from a group of Pseudomonas exotoxin (PE or a cytotoxic fragment or mutant thereof), Diptheria toxin or a cytotoxic fragment or mutant thereof, a botulinum toxin A through F, ricin or a cytotoxic fragment thereof, abrin or a cytotoxic fragment thereof, saporin or a cytotoxic fragment thereof, pokeweed antiviral toxin or a cytotoxic fragment thereof and bryodin 1 or a cytotoxic fragment thereof.

The present invention provides a method comprising causing or allowing binding of an antibody as provided herein to IL-4Rα. As noted, such binding may take place in vivo, e.g. following administration of an antibody, or nucleic acid encoding an antibody, or it may take place in vitro, for example in ELISA, Western blotting, immunocytochemistry, immuno-precipitation, affinity chromatography, and biochemical or cell based assays such as are described herein. The invention also provides for measuring levels of antigen directly, by employing an antibody according to the invention for example in a biosensor system.

The amount of binding of binding member to IL-4Rα may be determined. Quantification may be related to the amount of the antigen in a test sample, which may be of diagnostic interest. Screening for IL-4Rα binding and/or the quantification thereof may be useful, for instance, in screening patients for diseases or disorders associated with IL-4Rα, such as are referred to elsewhere herein. In one embodiment, among others, a diagnostic method of the invention comprises (i) obtaining a tissue or fluid sample from a subject, (ii) exposing said tissue or fluid sample to one or more antibodies of the present invention; and (iii) detecting bound IL-4Rα as compared to a control sample, wherein an increase in the amount of IL-4Rα binding as compared to the control may indicate an aberrant level of IL-4Rα expression or activity. Tissue or fluid samples to be tested include blood, serum, urine, biopsy material, tumours, or any tissue suspected of containing aberrant IL-4Rα levels. Subjects testing positive for aberrant IL-4Rα levels or activity may also benefit from the treatment methods disclosed later herein.

Those skilled in the art are able to choose a suitable mode of determining binding of the binding member to an antigen according to their preference and general knowledge, in light of the methods disclosed herein.

### IL-4Rα Antibody

This invention relates to antibodies for interleukin (IL)-4 receptor alpha (IL-4Rα, also referred to as CD 124), and their therapeutic use e.g. in treating or preventing disorders associated with IL-4Rα, IL-4 and/or IL-13, examples of which are asthma, COPD and inflammatory skin disorders, such as atopic dermatitis. See, e.g., U.S. Pat. No. 8,092,804, the entirety of which is incorporated by reference.

The human IL-4Rα subunit (Swiss Prot accession number P24394) is a 140 kDa type 1 membrane protein that binds human IL-4 with a high affinity (Andrews et al J. Biol. Chem. (2002) 277:46073-46078). The IL-4/IL-4Rα complex can dimerize with either the common gamma chain (γc, CD132) or the IL-13Ralpha1 (IL-13Rα1) subunit, via domains on IL-4, to create two different signalling complexes, commonly referred to as Type I and Type II receptors, respectively. Alternatively, IL-13 can bind IL-13Rα1 to form an IL-13/IL-13Rα1 complex that recruits the IL-4Rα subunit to form a Type II receptor complex. Thus, IL-4Rα mediates the biological activities of both IL-4 and IL-13 (reviewed by Gessner et al, Immunobiology, 201:285, 2000). In vitro studies have shown that IL-4 and IL-13 activate effector functions in a number of cell types, for example in T cells, B cells, eosinophils, mast cells, basophils, airway smooth muscle cells, respiratory epithelial cells, lung fibroblasts, and endothelial cells (reviewed by Steinke et al, Resp Res, 2:66, 2001, and by Willis-Karp, Immunol Rev, 202:175, 2004).

IL-4Rα is expressed in low numbers (100-5000 molecules/cell) on a variety of cell types (Lowenthal et al, J Immunol, 140:456, 1988), e.g. peripheral blood T cells, monocytes, airway epithelial cells, B cells and lung fibroblasts. The type I receptor predominates in hematopoietic cells, whereas the type II receptor is expressed on both hematopoietic cells and non-hematopoietic cells.

Antibodies to IL-4Rα have been described. Two examples are the neutralizing murine anti-IL-4Rα monoclonal antibodies MAB230 (clone 25463) and 16146 (clone 25463.11) which are supplied by R&D Systems (Minneapolis, Minn.) and Sigma (St Louis, Mo.), respectively. These antibodies are of the IgG2a subtype and were developed from mouse hybridomas developed from mice immunized with purified recombinant human IL-4Rα (baculovirus-derived). Two further neutralizing murine anti-IL-4Rα antibodies M57 and X2/45-12 are supplied by BD Biosciences (Franklin Lakes, N.J.) and eBioscience (San Diego, Calif.), respectively. These are IgG1 antibodies and are also produced by mouse hybridomas developed from mice immunized with recombinant soluble IL-4Rα.

Fully human antibodies are likely to be of better clinical utility than murine or chimeric antibodies. This is because human anti-mouse antibodies (HAMA) directed against the FC part of the mouse immunoglobulin are often produced, resulting in rapid clearance and possible anaphylactic reaction (Brochier et al., Int. J. Immunopharm., 17:41-48, 1995). Although chimeric antibodies (mouse variable regions and human constant regions) are less immunogenic than murine mAbs, human anti-Chimeric antibody (HACA) responses have been reported (Bell and Kamm, Aliment. Pharmacol. Ther., 14:501-514, 2000).

WO 01/92340 (Immunex) describes human monoclonal antibodies against IL-4 receptor generated by procedures involving immunization of transgenic mice with soluble IL-4R peptide and the creation of hybridoma cell lines that secrete antibodies to IL-4R, the principal antibody 12B5 is disclosed as being an IgG1 antibody and fully human. WO 05/047331 (Immunex) discloses further antibodies derived from 12B5 (renamed H1L1) via oligonucleotide mutagenesis of the VH region. Each mutated VH chain was paired with one of 6 distinct VL chains to create a small repertoire of antibody molecules.

WO 07/082,068 (Aerovance) discloses a method of treating asthma comprising administering a mutant human IL-4 protein having substitutions of R121D and Y124D. The specification teaches that such IL4 mutein administered in a pharmaceutical composition can antagonize the binding of wild type huIL-4 and wild type huIL-13 to receptors.

WO 08/054,606 (Regeneron) discloses particular antibodies against human IL-4R that were raised in transgenic mice capable of producing human antibodies.

There are advantages and benefits in the discovery and development of an antibody to human IL-4Rα that also exhibits cross-reactivity to the orthologous protein from another species, for example cynomolgus monkey. Such an antibody would facilitate the characterization of such antibodies with respect to pharmacology and safety in vivo. Potency or affinity to another species, which is for example less than 10-fold different than the human activity may be appropriate for such an evaluation. However, the human IL-4Rα protein displays a relatively little similarity to the orthologous IL-4Rα protein from other species except chimpanzee. Therefore, the discovery of high affinity and potency antibodies appropriate for clinical use with cross-reactivity to a species widely considered suitable for safety and toxicological evaluation for clinical development would be very challenging.

Through appropriately designed selection techniques and assays, the inventors have developed stable antibody compositions for IL-4Rα that inhibit the biological activity of human and cynomolgus monkey IL-4Rα.

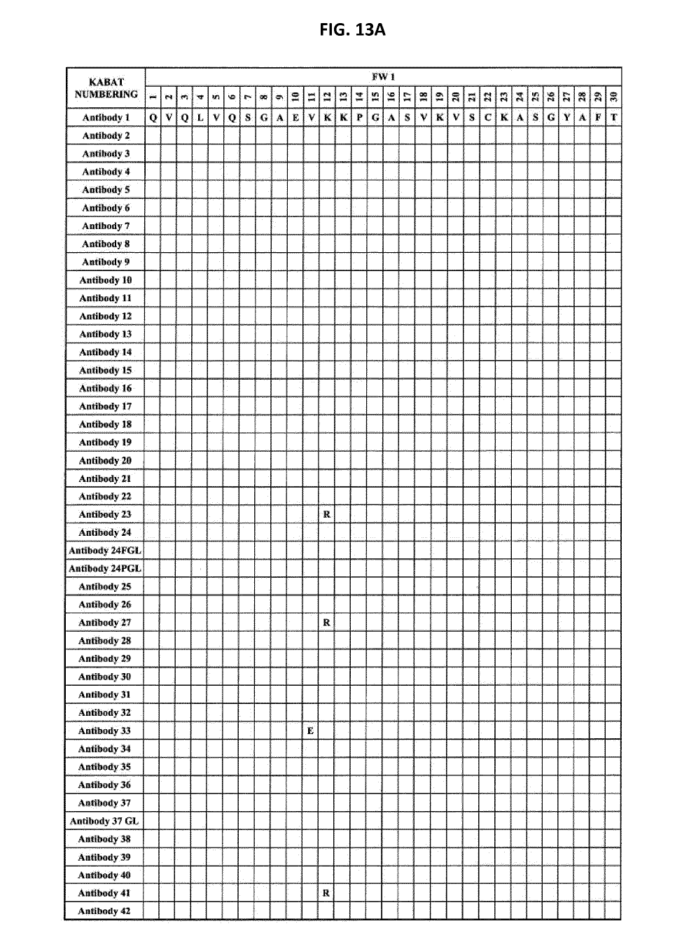

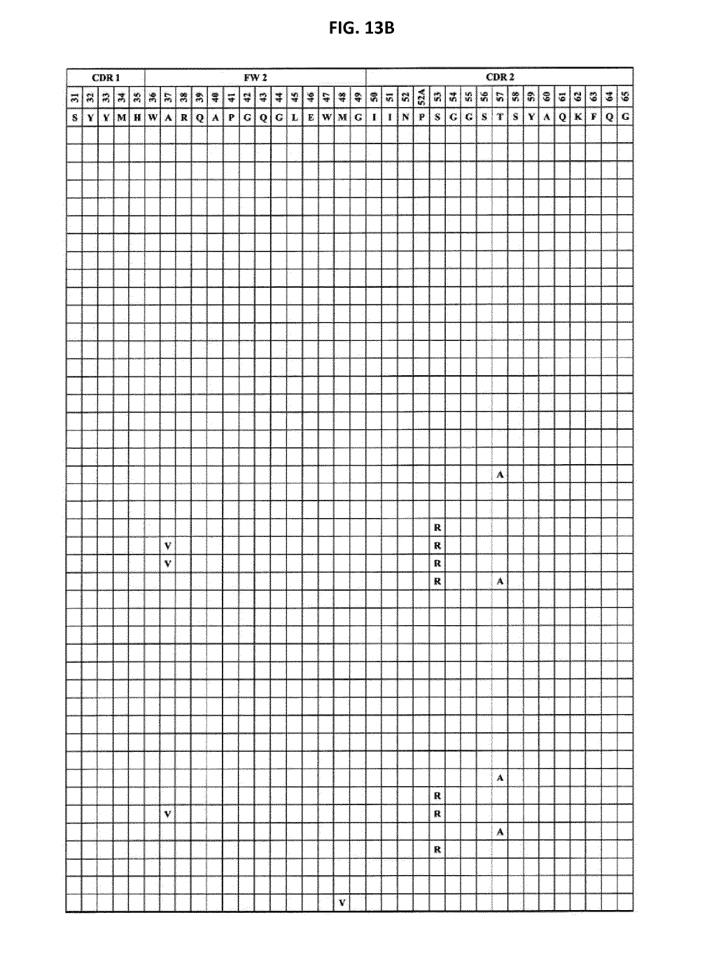

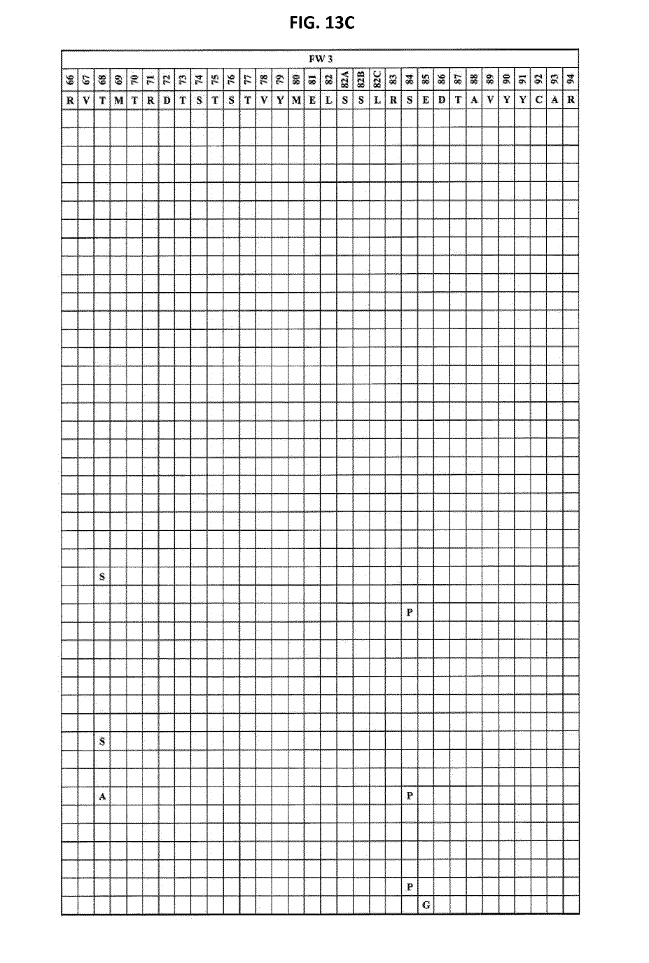

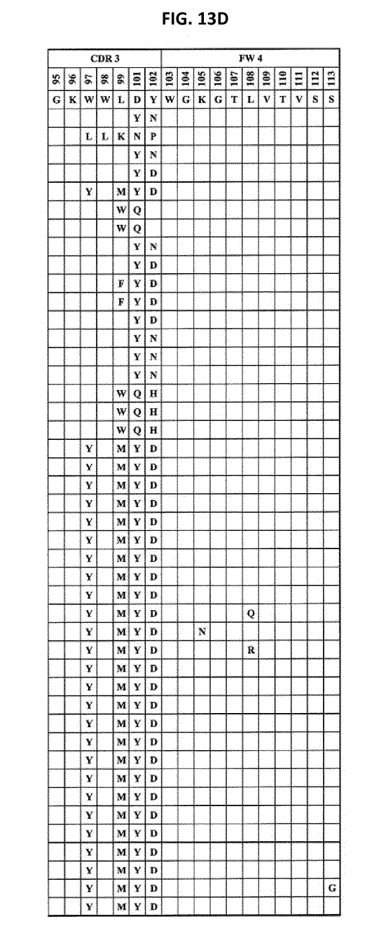

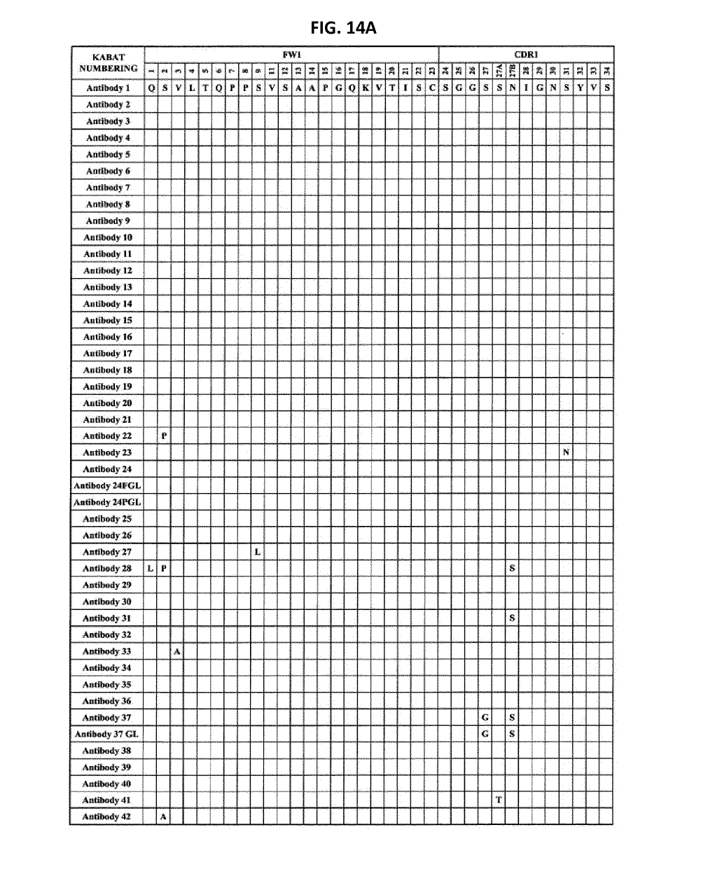

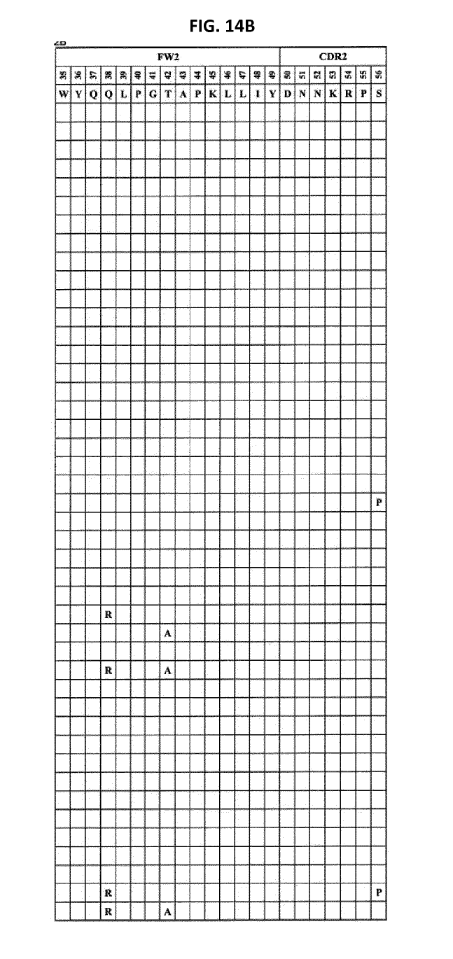

As detailed in U.S. Pat. No. 8,092,804, from an initial lead identification program a single antibody molecule to human IL-4Rα that also exhibited some, but weak, binding to and functional neutralization of, cynomolgus IL-4Rα was selected. Following a planned and defined process of targeted and random mutagenesis and further selection of mutants from this parent antibody molecule, a larger panel of antibody molecules with greatly improved properties was developed. VH and VL regions, including the complementarity determining regions (CDRs) of the parent antibody (Antibody 1), and of the optimized antibodies, are shown in FIGS. 13, 14, 15, and 16. These antibody molecules, VH, VL, and CDRs form aspects of the present invention.

In addition to wild-type IL-4Rα the antibodies of the present invention have also been found to bind 175V IL-4Rα, a common human variant.

Described herein are antibodies that neutralize the biological effects of IL-4Rα with high potency, bind IL-4Rα with high affinity and inhibit signalling induced by IL-4 and IL-13. Notably, the antibodies inhibit signalling from the high affinity complexes e.g. IL-4:IL-4Rα:γc, IL-4:IL-4Rα:IL-13Rα1, IL-13 IL-13Rα1:IL-4Rα. Such action prevents signalling of both IL-4 and IL-13. Additionally, the data indicate that the antibodies inhibit interaction and signalling of IL-4Rα type 1 and type 2 complexes. These and other properties and effects of the antibodies are described in further detail below.

The antibodies are useful for treating disorders in which IL-4Rα, IL-4 or IL-13 are expressed, e.g., one or more of the IL-4Rα-, IL-4- or IL-13-related disorders referred to elsewhere herein, such as asthma, COPD, or inflammatory skin disorders such as atopic dermatitis.

As described elsewhere herein, binding of an antibody to IL-4Rα may be determined using surface plasmon resonance e.g. BIAcore.

Surface plasmon resonance data may be fitted to a 1:1 Langmuir binding model (simultaneous ka kd) and an affinity constant KD calculated from the ratio of rate constants kd1/ka1. An antibody of the invention may have a monovalent affinity for binding human IL-4Rα that is less than 20 nM. In other embodiments the monovalent affinity for binding human IL-4Rα that is less than 10 nM, e.g. less than 8, less than 5 nM. In other embodiments the binding member also binds cynomolgus IL-4Rα. In one embodiment, an antibody of the present invention has a monovalent affinity for binding human IL-4Rα in the range 0.05 to 12 nM. In one embodiment, an antibody of the present invention has a monovalent affinity for binding human IL-4Rα in the range of 0.1 to 5 nM. In one embodiment, an antibody of the present invention has a monovalent affinity for binding human IL-4Rα in the range of 0.1 to 2 nM.

In one embodiment, an antibody of the invention may immunospecifically bind to human IL-4Rα and may have an affinity (KD) of less than 5000 pM, less than 4000 pM, less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method described herein or known to one of skill in the art (e.g., a BIAcore assay, ELISA) (Biacore International AB, Uppsala, Sweden).

In one embodiment, an anti-IL-4Rα antibody of the invention may immunospecifically bind to bind to human IL-4Rα and may have an affinity (KD) of 500 pM, 100 pM, 75 pM or 50 pM as assessed using a method described herein or known to one of skill in the art (e.g., a BIAcore assay, ELISA).

In some embodiments, antibodies according to the invention can neutralize IL-4Rα with high potency. Neutralization means inhibition of a biological activity mediated by IL-4Rα. Antibodies of the invention may neutralize one or more activities mediated by IL-4Rα. The inhibited biological activity is likely mediated by prevention of IL-4Rα forming a signalling complex with gamma chain (or IL-13Rα) and either of the associated soluble ligands, e.g. IL-4 or IL-13.

Neutralization of IL-4 or IL-13 signalling through its IL-4Rα containing receptor complex may be measured by inhibition of IL-4 or IL-13 stimulated TF-1 cell proliferation.

The epitope of human IL4Rα to which the antibodies of the invention bind was located by a combination of mutagenesis and domain swapping. Whole domain swap chimeras localized the epitope to domain 1 (D1) of human IL4Rα (residues M1-E119). Human IL-4Rα contains five loop regions, which are in close proximity to IL4 in a crystal structure (Hage et al., Cell 97:271-281, 1999). Loop swap chimeras enabled the further localization of the human IL-4Rα epitope bound by an antibody of the invention, to a major component in loop 3 (residues L89-N98) and a minor component in loop 2 (residues V65-H72). Chimeras without human loop 3 failed to inhibit human IL-4Rα binding to antibody and chimeras without loop 2 gave a 100 fold higher IC₅₀ than human IL-4Rα (Table 5). Consistent with the domain swap data both loop2 and loop3 are located in domain 1 (D1) (Hage et al., Cell 97:271-281, 1999).

The antibody epitope was located to a discontinuous epitope of 18 amino acids in two loop regions of human IL-4Rα; V65-H72 and L89-N98. The epitope can be further localized to amino acid residues L67 and L68 of loop 2 and D92 and V93 of loop3 (see SEQ ID NO: 454 or 460 for location of residues 67, 68, 92 and 93). The D92 residues was the most important, followed by V93, for the antibody tested was still capable of binding chimeric IL-4Rα that lacked the L67 and/or L68 residues in loop2. Of course it is likely that the antibodies of the invention will also bind residues of the human IL-4Rα protein in addition to one of L67, L68, D92 and V93.

According to one aspect of the invention there is provided an antibody formulation comprising an antibody capable of binding to human interleukin-4 receptor alpha (hIL-4Rα) at least one amino acid residue selected from the amino acid at position 67, 68, 92 and 93, according to the position in SEQ ID NO: 460. According to one aspect of the invention there is provided an antibody formulation comprising an isolated binding member, e.g., antibody, capable of binding to at least one of amino acid residues 67, 68, 92 and 93, according to the position in SEQ ID NO: 460, of native human interleukin-4 receptor alpha (hIL-4Rα). In a particular embodiment the isolated binding member, e.g., antibody, is capable of binding to the amino acid at position 92 of hIL-4Rα, according to the position in SEQ ID NO: 460. In another embodiment the isolated binding member, e.g., antibody, is capable of binding to D92 and at least one other residue selected from L67, L68 and V93. In another embodiment the isolated binding member, e.g., antibody, is capable of binding to D92 and V93. In another embodiment the isolated binding member, e.g., antibody, is capable of binding to D92, V93 and either of L67 or L68. In another embodiment the antibody is capable of binding to each of L67, L68, D92 and V93. Each of these embodiments refers to amino acid positions in hIL-4Rα whose locations can be identified according to the hIL-4Rα amino acid sequence (from positions 1-229) depicted in SEQ ID NO: 460. In one embodiment the binding member, e.g., antibody, is able to bind to the recited epitope residues (i.e. at least one of positions 67, 68, 92 and 93) of full-length hIL-4Rα. In one embodiment the binding member, e.g., antibody, is able to bind to the recited epitope residues (i.e. at least one of positions 67, 68, 92 and 93) of native hIL-4Rα expressed on the cell surface. In one embodiment the binding member, e.g., antibody, is able to bind to the recited epitope residues (i.e. at least one of positions 67, 68, 92 and 93) of recombinantly expressed full-length (229 amino acid) hIL-4Rα.

According to a further aspect of the invention there is provided an antibody formulation comprising an isolated binding member, e.g., antibody, capable of binding human interleukin-4 receptor alpha (hIL-4Rα). In a particular embodiment the binding member is a human antibody. In a further embodiment the binding member is also capable of binding cynomolgus monkey interleukin-4 receptor alpha (cyIL-4Rα).

According to another aspect of the invention there is provided an antibody formulation comprising an isolated binding member for human interleukin-4 receptor alpha (hIL-4Rα), which binding member has an IC₅₀ geomean for inhibition of human IL-4 (hIL-4) induced cell proliferation of less than 50 pM in TF-1 proliferation assay using 18 pM soluble human IL-4 protein and which binding member is also capable of binding cyIL-4Rα.

In particular embodiments of this aspect of the invention the binding member has an IC₅₀ geomean for inhibition of human IL-4 (hIL-4) induced cell proliferation of less than 50 pM, less than 35 pM, less than 25 pM, or less than 20 pM, in a TF-1 proliferation assay using 18 pM of soluble human IL-4. In particular embodiments the binding member of the invention has an IC₅₀ geomean for inhibition of human IL-4 (hIL-4) induced cell proliferation of between 1 to 50 pM, 1 to 35 pM, 2 to 30 pM, 2 to 25 pM, 2 to 12 pM, using 18 pM of soluble human IL-4 in a method described herein (e.g. Example 3.2.1) or known to one of skill in the art. Binding to cyIL-4Rα can be measured by any suitable means.

Similarly, antibodies within the scope of the invention have an IC₅₀ geomean for inhibition of human IL-13 (hIL-13)-mediated TF-1 proliferation (via neutralization of hIL-4Rα) of less than 200 pM using 400 pM soluble human IL-13 (hIL-13). In a particular embodiment the IC₅₀ geomean for inhibition of human IL-13 (hIL-13)-mediated TF-1 proliferation (via neutralization of hIL-4Rα) using 400 pM soluble human IL-13 (hIL-13) is between 5 and 75 pM or between 5 and 45 pM.

In particular embodiments, the antibody formulations of the invention comprise antibodies substantially incapable of binding to murine IL-4Rα. By this we mean that an antibody of the invention is capable of at least 500-fold (such as at least 500-fold, at least 1000-fold, at least 1500-fold, at least 2000-fold, at least 3000-fold, at least 4000-fold) greater binding to human interleukin-4 receptor alpha than to murine IL-4Rα (i.e. binding to murine IL-4Rα is at least 500 fold weaker than to human IL-4Rα). This can be measured, for example, by the HTRF competition assay.

Inhibition of biological activity may be partial or total. In specific embodiments, antibodies are provided that inhibit IL-4Rα biological activity by at least 95%, at least 90%, at least 85%, at least 80%, at least 75%, at least 70%, at least 60%, or at least 50% of the activity in the absence of the binding member. The degree to which an antibody neutralizes IL-4Rα is referred to as its neutralizing potency. Potency may be determined or measured using one or more assays known to the skilled person and/or as described or referred to herein. For example, potency may be assayed in:
Receptor-ligand binding assays in fluorescent (e.g. HTRF or DELFIA) or radioactive format
Fluorescent (e.g. HTRF or DELFIA) epitope competition assay
Cell-based functional assays including STATE Phosphorylation of human or cynomolgous PBMCs, proliferation of TF-1 cells, eotaxin release from human or cynomolgous fibroblast cell lines, VCAM-1 upregulation on human endothelial vein cells or proliferation of human T-cells.

Some of these assays methods are also described in the Examples of U.S. Pat. No. 8,092,804.

Neutralizing potency of an antibody as calculated in an assay using IL-4Rα from a first species (e.g. human) may be compared with neutralizing potency of the binding member in the same assay using IL-4Rα from a second species (e.g. cynomolgus monkey), in order to assess the extent of cross-reactivity of the binding member for IL-4Rα of the two species. There are great advantages in having an antibody which binds both the human target and the orthologous target from another species. A key advantage arises when the binding-member is being advanced as a therapeutic product and safety studies (e.g. toxicity) need to be conducted in another species. Potency or affinity to another species, which is for example less than 10-fold different than the human activity may be appropriate for such an evaluation.

According to a particular embodiment of the present invention, the ratio of binding of the binding member, e.g., antibody, when as a scFv to hIL-4Rα and to cyIL-4Rα measured using the receptor-ligand binding assay is at least 6:1. As used here, “at least” 6:1, includes 8:1, 10:1 etc; rather than 2:1, 1:1.

Antibodies of the invention bind human IL-4Rα and cynomolgus monkey IL-4Rα, and may have a less than 250-fold, e.g. less than 150-, 100-, 75-, 50-, 25-, 20-, 15-, 10-fold difference in potency for neutralizing human and cynomolgus IL-4Rα as determined in the receptor-ligand binding assay, with the binding member being in scFv format, as in U.S. Pat. No. 8,092,804.

In some embodiments, neutralization potency of antibodies of the invention (when in scFv format) for human and cynomolgus IL-4Rα measured using the receptor-ligand binding assay is within 25-fold. In one embodiment the neutralization potency of antibodies of the invention for human and cynomolgus IL-4Rα is within 210-fold; i.e., binding to human IL-4Rα is no greater than 210-fold that against cynomologous IL-4Rα. In another embodiment, said neutralization potency is between 5:1 and 210:1, such as between 5:1 and 100:1.

For functional cell-based assays potency is normally expressed as an IC₅₀ value, in nM unless otherwise stated. In functional assays, IC₅₀ is the molar concentration of a binding member that reduces a biological (or biochemical) response by 50% of its maximum. IC₅₀ may be calculated by plotting % of maximal biological response as a function of the log of the binding member concentration, and using a software program such as Prism (GraphPad) or Origin (Origin Labs) to fit a sigmoidal function to the data to generate IC₅₀ values.

For receptor-ligand binding assays, potency is normally expressed as Ki (the inhibition constant), the concentration of binding member that would occupy 50% of receptors if no labeled ligand were present. Whereas IC₅₀ may vary between experiments depending on ligand concentration, the Ki is an absolute value calculated from the Cheng Prusoff equation.

An antibody of the invention may have a neutralizing potency or Ki of up to 5 nM in a human IL-4RαHTRF® assay as described herein. This assay can be used to determine Ki for antibodies in scFv format. The Ki may for example be up to 5.0, 4.0, 3.0, 2.0, 1.0, 0.5, 0.2, 0.1, 0.05, or 0.02 nM.

Additionally, binding kinetics and affinity (expressed as the equilibrium dissociation constant, KD) of IL-4Rα antibodies for IL-4Rα may be determined, e.g. using surface plasmon resonance such as BIAcore®, or Kd may be estimated from pA₂ analysis.

In some embodiments, the antibodies of the invention are capable of binding to glycosylated hIL-4Rα.

In some embodiments, antibodies of the invention may optionally be specific for IL-4Rα over other structurally related molecules (e.g. other interleukin receptors) and thus bind IL-4Rα selectively. For example, antibodies of the invention may not cross-react with any of IL-13Rα1 or IL-13Rα2 and the common gamma chain (γc).

An antibody of the invention may comprise an antibody molecule, e.g. a human antibody molecule. The binding member comprises an antibody VH and/or VL domain. VH domains of antibodies are also provided as part of the invention. Within each of the VH and VL domains are complementarity determining regions, (“CDRs”), and framework regions, (“FRs”). A VH domain comprises a set of HCDRs, and a VL domain comprises a set of LCDRs. An antibody molecule may comprise an antibody VH domain comprising a VH CDR1, CDR2 and CDR3 and a framework. It may alternatively or also comprise an antibody VL domain comprising a VL CDR1, CDR2 and CDR3 and a framework. A VH or VL domain framework comprises four framework regions, FR1, FR2, FR3 and FR4, interspersed with CDRs in the following structure:
FR1-CDR1-FR2-CDR2-FR3-CDR3-FR4.

Examples of antibody VH and VL domains, FRs and CDRs according to the present invention are as listed in the appended sequence listing that forms part of the present disclosure. All VH and VL sequences, CDR sequences, sets of CDRs and sets of HCDRs and sets of LCDRs disclosed herein represent aspects and embodiments of the invention. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “full set of CDRs” includes HCDRs and LCDRs. Typically, antibodies of the invention are monoclonal antibodies.

A further aspect of the invention is an antibody molecule comprising a VH domain that has at least 75, 80, 85, 90, 95, 98 or 99% amino acid sequence identity with a VH domain of any of Antibodies 1 to 42 shown in the appended sequence listing, and/or comprising a VL domain that has at least 75, 80, 85, 90, 95, 98 or 99% amino acid sequence identity with a VL domain of any of Antibodies 1 to 42 shown in the appended sequence listing. Accelerys' “MacVector™” program may be used to calculate % identity of two amino acid sequences.

An antibody of the invention may comprise an antigen-binding site within a non-antibody molecule, normally provided by one or more CDRs e.g. an HCDR3 and/or LCDR3, or a set of CDRs, in a non-antibody protein scaffold, as discussed further below.

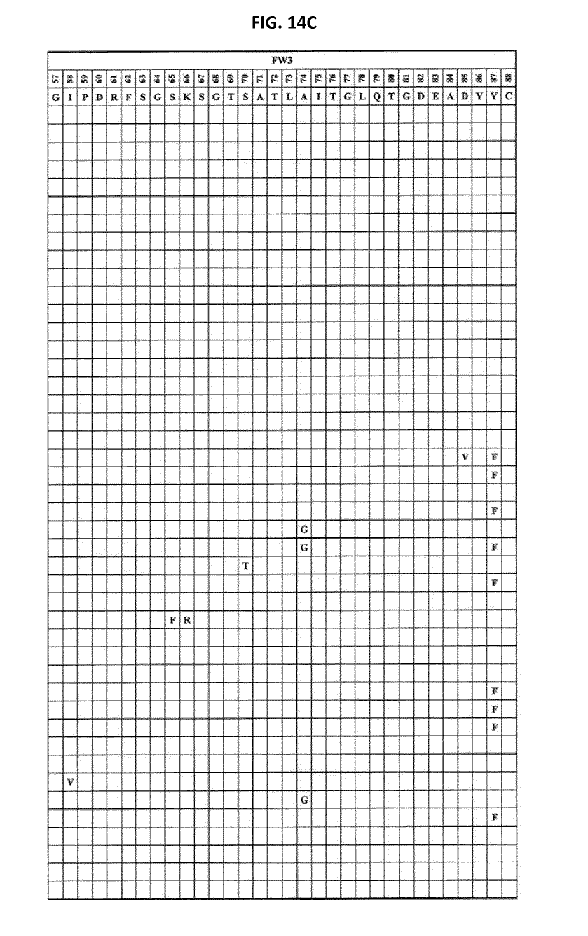

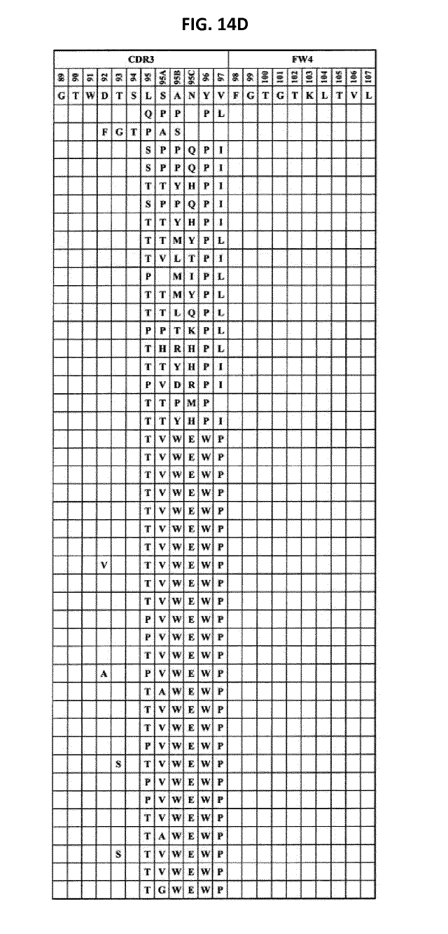

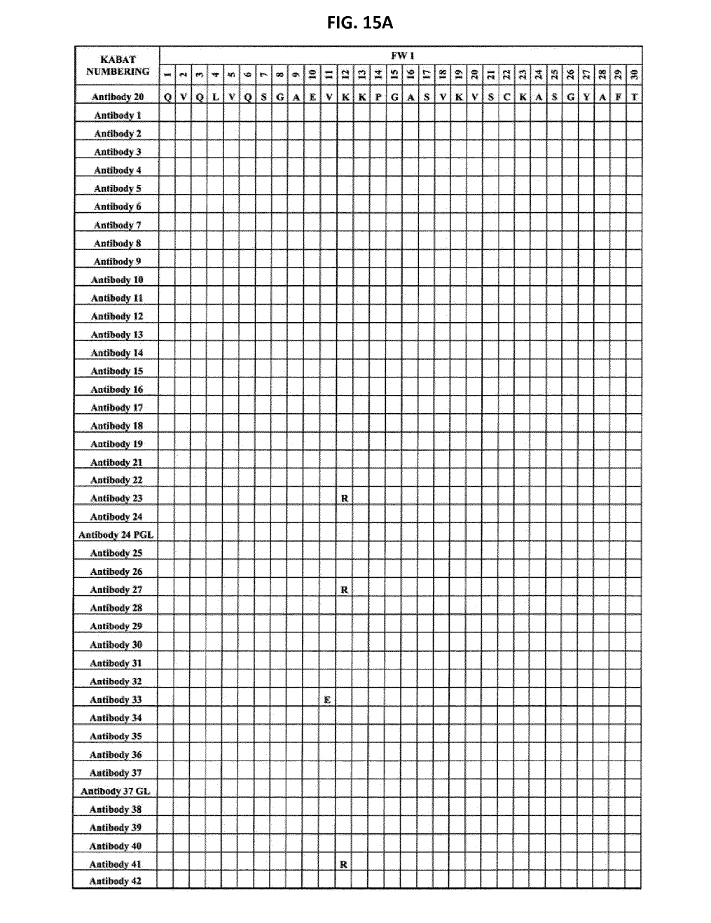

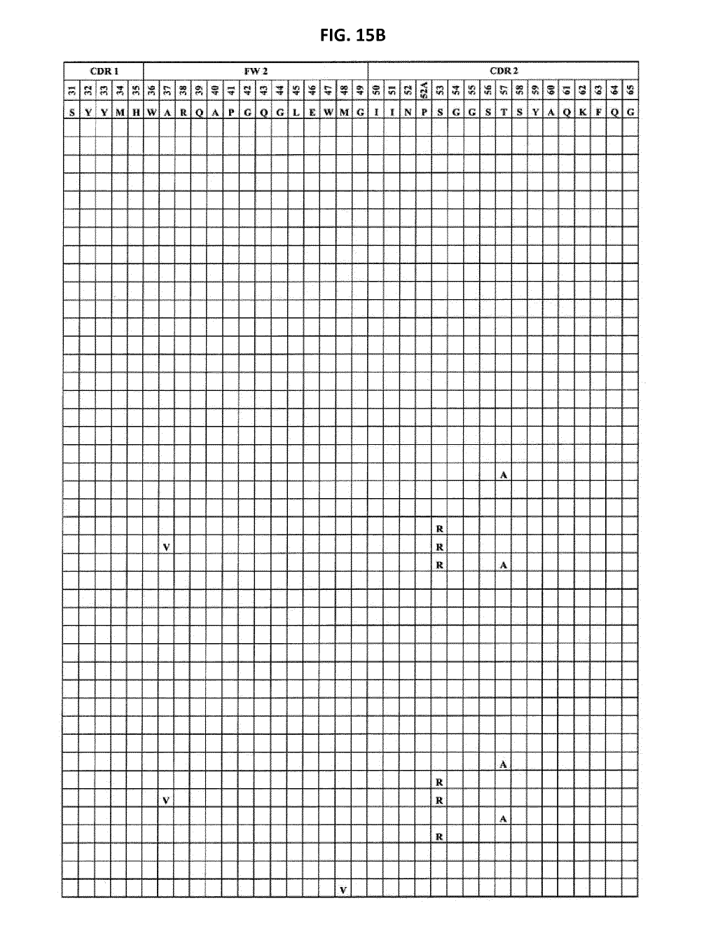

The inventors isolated a parent antibody molecule (Antibody 1) with a set of CDR sequences as shown in FIGS. 13 (VH domain) and 14 (VL domain). Through a process of optimization they generated a panel of antibody clones, including those numbered 2 to 20, with CDR3 sequences derived from the parent CDR3 sequences and having substitutions at the positions indicated in FIG. 13 (VH domain) and FIG. 14 (VL domain). Thus for example, it can be seen from FIG. 13 (A to D), that Antibody 2 has the parent HCDR1, HCDR2, LCDR1 and LCDR2 sequences, and has the parent LCDR3 sequence in which Kabat residue 95 is replaced by Q, Kabat residue 95A, 95B and 96 are each replaced by P and Kabat residue 97 is replaced by L; and has parent HCDR3 sequence in which Kabat residue 101 is replaced by Y and Kabat residue 102 is replaced by N.

The parent antibody molecule, and Antibody molecules 2 to 20, as described herein refer respectively to antibody molecules with CDRs of the parent antibody molecule and to antibody molecules with CDRs of antibody molecules 2 to 20. Through a further process of optimization the inventors generated a panel of antibody clones numbered 21-42, with additional substitutions throughout the VH and VL domains. Thus, for example, Antibody 21 has the same LCDR1, LCDR2, LCDR3, HCDR1, and HCDR3 as Antibody 20; it has the parent HCDR2 sequence of Antibody 20 but with Kabat residue 57 replaced by A; and it has Kabat residues 85 and 87 (in LFW3) replaced by V and F, respectively.

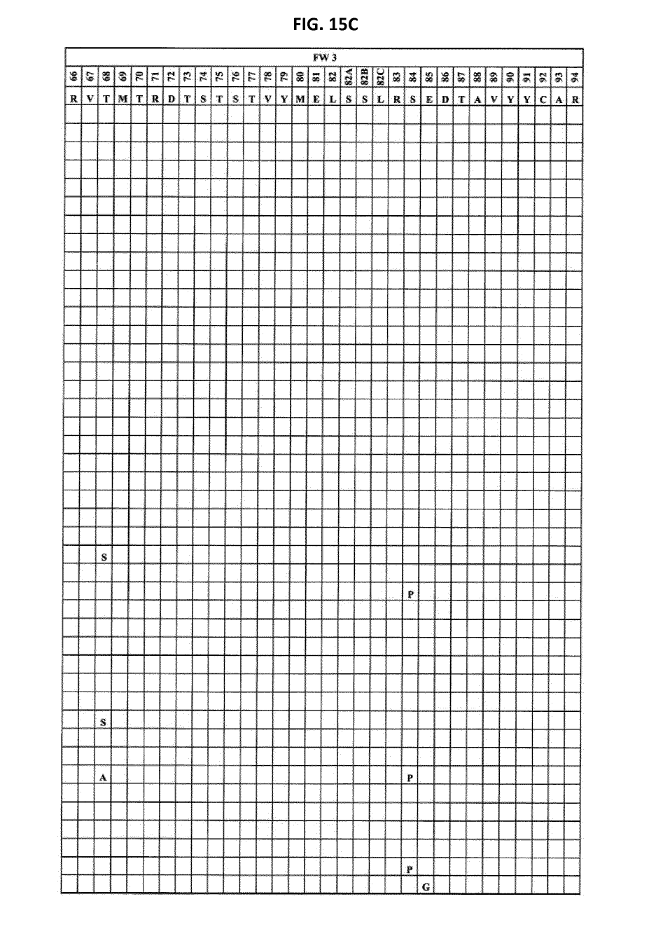

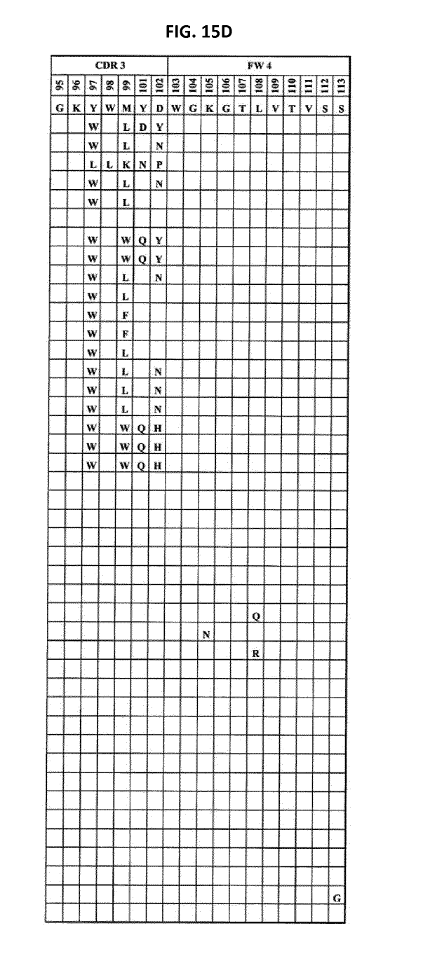

Described herein is a reference binding member comprising the Antibody 20 set of CDRs as shown in FIGS. 15 (VH) and 16 (VL), in which HCDR1 is SEQ ID NO: 193 (Kabat residues 31-35), HCDR2 is SEQ ID NO: 194 (Kabat residues 50-65), HCDR3 is SEQ ID NO: 195 (Kabat residues 95-102), LCDR1 is SEQ ID NO: 198 (Kabat residues 24-34), LCDR2 is SEQ ID NO: 199 (Kabat residues 50-56) and LCDR3 is SEQ ID NO: 200 (Kabat residues 89-97). Further antibodies can be described with reference to the sequence in the reference binding member.

The antibody formulation comprising an antibody may comprise one or more CDRs (i.e. at least one, at least 2, at least 3, at least 4 at least 5 and at least 6) as described herein, e.g. a CDR3, and optionally also a CDR1 and CDR2 to form a set of CDRs. The CDR or set of CDRs may be a parent CDR or parent set of CDRs, or may be a CDR or set of CDRs of any of Antibodies 2 to 42, or may be a variant thereof as described herein.

For example, an antibody or a VL domain according to the invention may comprise the reference LCDR3 with one or more of Kabat residues 92-97 substituted for another amino acid. Exemplary substitutions include:
Kabat residue 92 replaced by Phe (F), Val (V) or Ala (A);
Kabat residue 93 replaced by Gly (G) or Ser (S);
Kabat residue 94 replaced by Thr (T)
Kabat residue 95 replaced by Leu (L), GLn (Q), Pro (P) or Ser (S);
Kabat residue 95a replaced by Ser (S), Prol (P), Ala (A), Thr (T), H is (H) or Gly (G);
Kabat residue 95b replaced by Ala (A), Pro (P), Ser (S), Tyr (Y), Met (M), Leu (L), Thr (T), Arg (R) or Asp (D);
Kabat residue 95c replaced by Asn (N), Gln (Q), H is (H), Tyr (Y), Thr (T), Be (I), Lys (K), Arg (R) or Met (M);
Kabat residue 96 replaced by Tyr (Y) or Pro (P);
Kabat residue 97 replaced by Val (V), Leu (L) or Ile (I).

An antibody or a VH domain may comprise the reference HCDR3 with one or more of Kabat residues 97-102 substituted for another amino acid. Exemplary substitutions include:
Kabat residue 97 replaced by Trp (W) or Leu (L);
Kabat residue 98 replaced by Leu (L);
Kabat residue 99 replaced by Leu (L), Lys (K), Phe (F) or Trp (W);
Kabat residue 101 replaced by Asp (D), Asn (N) or Gln (Q);
Kabat residue 102 replaced by Tyr (Y), Asn (N), Pro (P) or H is (H).

Antibodies of the invention may comprise an HCDR1, HCDR2 and/or HCDR3 of any of Antibodies 1 to 42 and/or an LCDR1, LCDR2 and/or LCDR3 of any of Antibodies 1 to 42. An antibody may comprise a set of VH CDRs of one of these antibodies. Optionally it may also comprise a set of VL CDRs of one of these antibodies, and the VL CDRs may be from the same or a different antibody as the VH CDRs. A VH domain comprising a set of HCDRs of any of Antibodies 1 to 42, and/or a VL domain comprising a set of LCDRs of any of Antibodies 1 to 42, are also individual embodiments of the invention.

Typically, a VH domain is paired with a VL domain to provide an antibody antigen-binding site, although as discussed further below a VH or VL domain alone may be used to bind antigen. In one embodiment, the Antibody 1 VH domain is paired with the Antibody 1 VL domain, so that an antibody antigen-binding site is formed comprising both the Antibody 1 VH and VL domains. Analogous embodiments are provided for the other VH and VL domains disclosed herein. In other embodiments, the Antibody 1 VH is paired with a VL domain other than the antibody 1 VL. Light-chain promiscuity is well established in the art. Again, analogous embodiments are provided by the invention for the other VH and VL domains disclosed herein. Thus, the VH of the parent (Antibody 1) or of any of Antibodies 2 to 42 may be paired with the VL of the parent or of any of Antibodies 2 to 42.

One aspect of the invention is an antibody comprising a VH and VL domain wherein the VH domain comprises a sequence disclosed in FIG. 13 or 15.

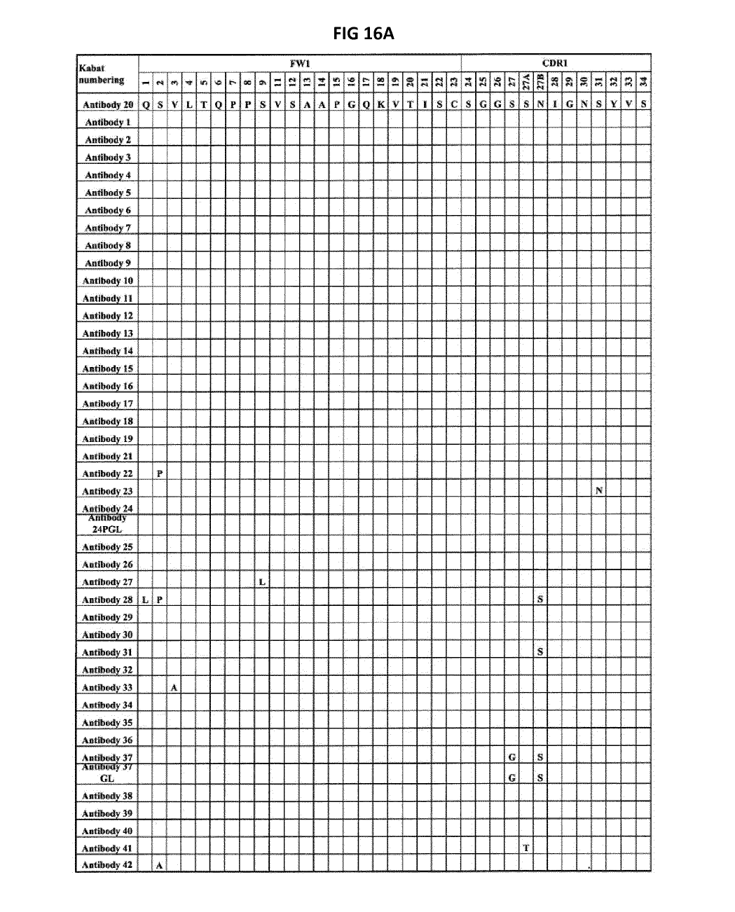

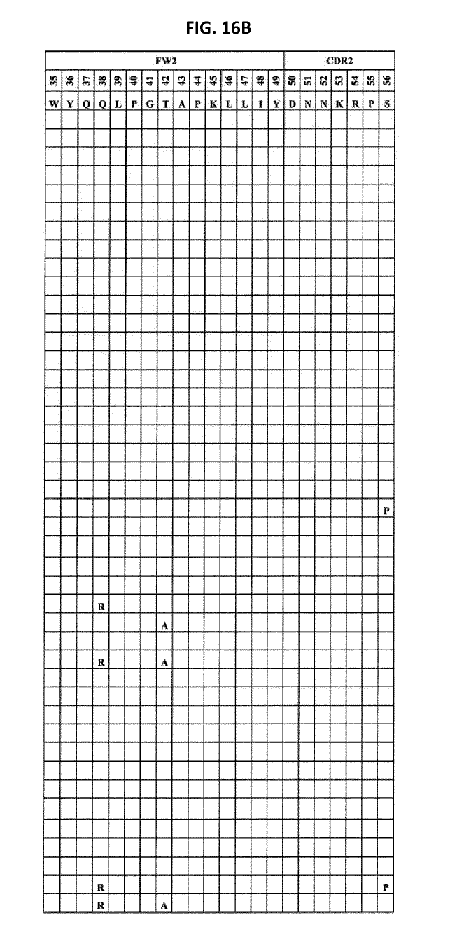

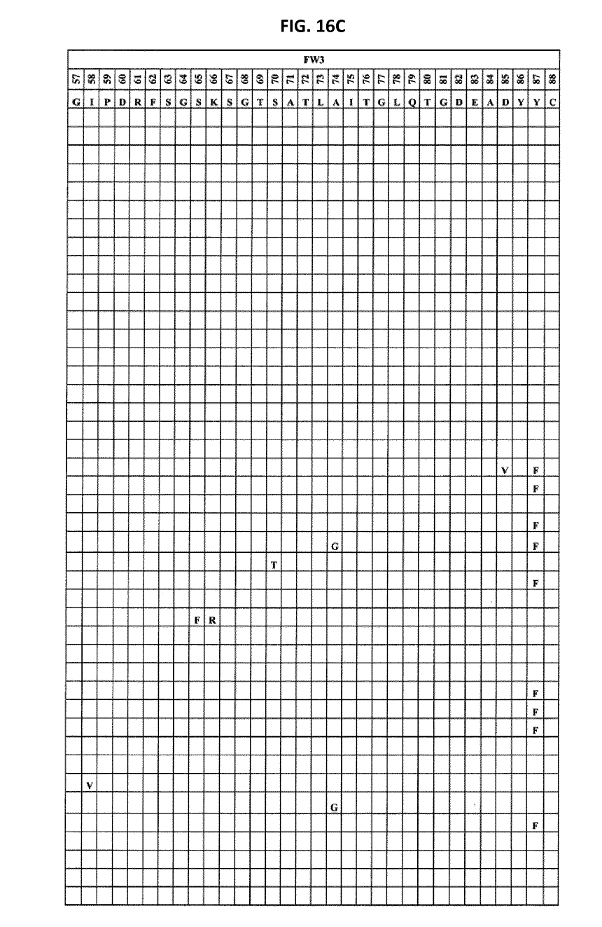

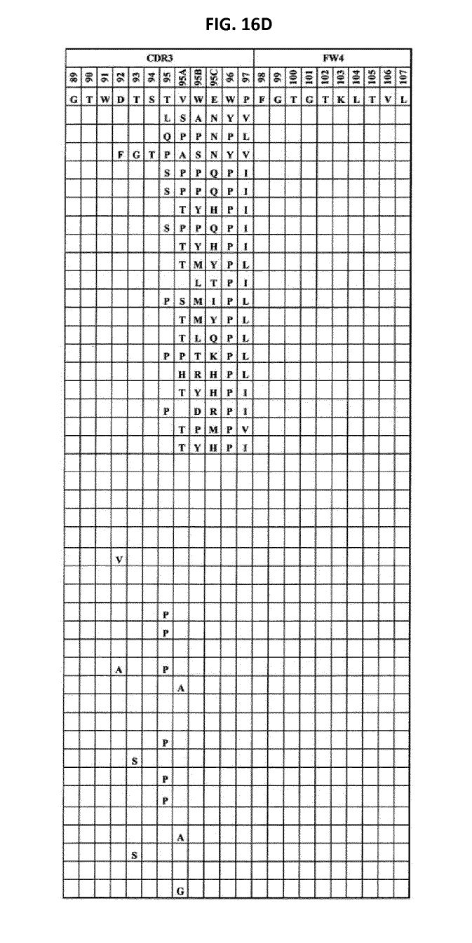

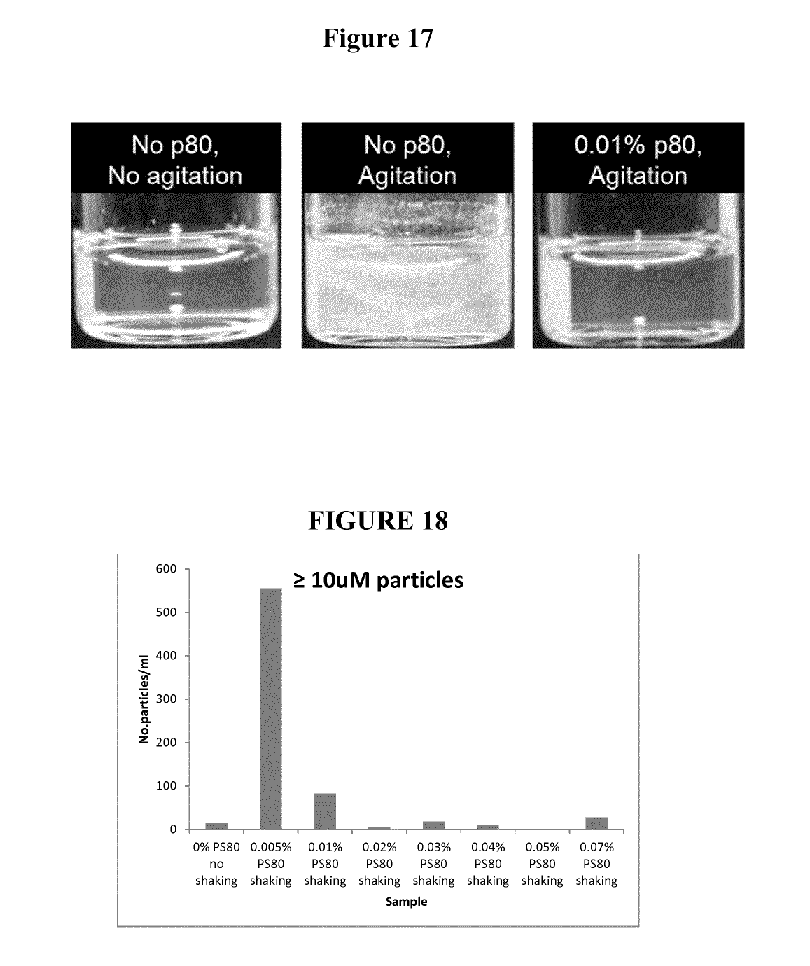

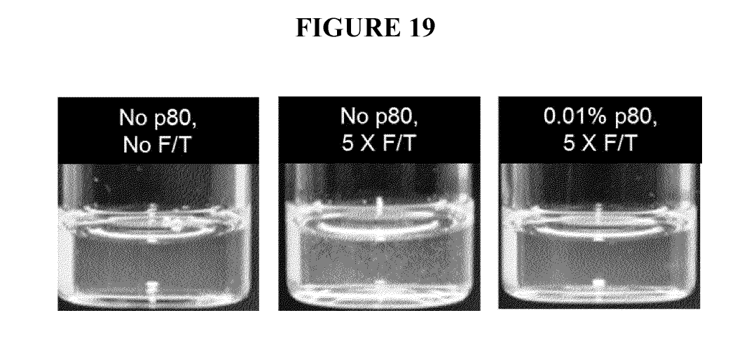

Another aspect of the invention is an antibody comprising a VH and VL domain wherein the VL domain comprises a sequence disclosed in FIG. 14 or 16.

Another aspect of the invention is an isolated antibody molecule comprising a VH domain with the VH domain amino acid sequence shown in SEQ ID NO: 362, 442, 232, 422 or 432 and a VL domain with the VL domain amino acid sequence shown in SEQ ID NOs: 367, 237, 447, 437 or 427.

An antibody may comprise a set of H and/or L CDRs of the parent antibody or any of Antibodies 2 to 42 with twelve or ten or nine or fewer, e.g. one, two, three, four or five, substitutions within the disclosed set of H and/or L CDRs. For example, an antibody of the invention may comprise the Antibody 16 or Antibody 20 set of H and/or L CDRs with 12 or fewer substitutions, e.g. seven or fewer substitutions, e.g. zero, one, two, three, four, five, or six substitutions. Substitutions may potentially be made at any residue within the set of CDRs, and may be within CDR1, CDR2 and/or CDR3.

Thus, according to one aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, wherein the set of CDRs has 12 or fewer amino acid alterations from a reference set of CDRs in which:
HCDR1 has amino acid sequence SEQ ID NO: 153;
HCDR2 has amino acid sequence SEQ ID NO: 154;
HCDR3 has amino acid sequence SEQ ID NO: 155;
LCDR1 has amino acid sequence SEQ ID NO: 158;
LCDR2 has amino acid sequence SEQ ID NO: 159; and
LCDR3 has amino acid sequence SEQ ID NO: 160.

The reference antibody in this instance is Antibody 16.

The isolated binding member may have 10 or fewer, 8 or fewer, 7 or fewer, e.g. 6, 5, 4, 3, 2, 1 or 0 amino acid alterations from the reference set of CDRs. Particular alterations are amino acid substitutions.

According to another aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, wherein the set of CDRs has 12 or fewer amino acid alterations from a reference set of CDRs in which:
HCDR1 has amino acid sequence SEQ ID NO: 193;
HCDR2 has amino acid sequence SEQ ID NO: 194;
HCDR3 has amino acid sequence SEQ ID NO: 195;
LCDR1 has amino acid sequence SEQ ID NO: 198;
LCDR2 has amino acid sequence SEQ ID NO: 199; and
LCDR3 has amino acid sequence SEQ ID NO: 200.

The reference antibody in this instance is Antibody 20.

The isolated binding member may have 10 or fewer, 8 or fewer, 7 or fewer e.g. 6, 5, 4, 3, 2, 1 or 0 amino acid alterations from the reference set of CDRs. Particular alterations are amino acid substitutions. In a particular embodiment, the isolated binding member has 4 or fewer amino acid substitutions from the reference set of CDRs identified above.

According to another aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, wherein the set of CDRs has 6 or fewer amino acid alterations from a reference set of CDRs in which:
HCDR1 has amino acid sequence SEQ ID NO: 363;
HCDR2 has amino acid sequence SEQ ID NO: 364;
HCDR3 has amino acid sequence SEQ ID NO: 365;
LCDR1 has amino acid sequence SEQ ID NO: 368;
LCDR2 has amino acid sequence SEQ ID NO: 369; and
LCDR3 has amino acid sequence SEQ ID NO: 370.

The reference antibody in this instance is Antibody 37.

Substitutions may be within CDR3, e.g. at the positions substituted in any of Antibodies 2 to 42, as shown in FIG. 13 or 15 (VH domain) and 14 or 16 (VL domain). Thus, the one or more substitutions may comprise one or more substitutions at the following residues:
Kabat residue 97, 98, 99, 101 or 102 in HCDR3; or
Kabat residue 92, 93, 94, 95, 95A, 95B, 95C, 96 or 97 in LCDR3.

Thus, a CDR3 may for example be a reference LCDR3 having one or more substitutions at Kabat residues 92, 93, 94, 95, 95A, 95B, 95C, 96 or 97.

Examples of substitutions in parent/reference CDRs are described elsewhere herein. As described, the substitutions may comprise one or more substitutions as shown in FIGS. 13 to 16.

An antibody of the invention may comprise the HCDR1, HCDR2 and/or HCDR3 of the reference Antibody 20, or with one or more of the following substitutions:
HCDR2 wherein Kabat residue 53 is Arg (R);
HCDR2 wherein Kabat residue 57 is Ala (A);
HCDR3 wherein Kabat residue 97 is Trp (W) or Leu (L); Kabat residue 98 is Leu;
Kabat residue 99 is Leu (L), Lys (K) or Trp (W); Kabat residue 101 is Asn (N) or Gln (Q); and/or Kabat residue 102 is Tyr (Y), Asn (N), Pro (P) or H is (H).

An antibody of the invention may comprise an LCDR1, LCDR2 and/or LCDR3 of the reference Antibody 20, or with one or more of the following substitutions:

LCDR1 wherein Kabat residue 27 is Gly (G);
Kabat residue 27A is Thr (T);
Kabat residue 27B is Ser (S);
Kabat residue 31 is Asn (N);
LCDR2 wherein Kabat residue 56 is Pro (P);
LCDR3 wherein Kabat residue 92 is Phe (F), Val (V) or Ala (A);
Kabat residue 93 is Gly (G) or Ser (S);
Kabat residue 94 is Thr (T);
Kabat residue 95 is Leu (L), Gln (Q), Pro (P) or Ser (S);
Kabat residue 95A is Ser (S), Pro (P), Ala (A), Thr (T), H is (H) or Gly (G);
Kabat residue 95B is Ala (A), Pro (P), Ser (S), Tyr (Y), Met (M), Leu (L), Thr (T), Asp (D) or Arg (R);
Kabat residue 95C is Asn (N), Gln (Q), H is (H), Tyr (Y), Ile (I), Lys (K), Arg (R), Thr (T) or Met (M);
Kabat residue 96 is Tyr (Y) or Pro (P);
and/or Kabat residue 97 is Val (V), Leu (L) or Ile (I).

In a particular embodiment, with reference to Antibody 20 sequence, Kabat residue 53 in HCDR2 is replaced by Arg (R); and/or Kabat residue 57 in HCDR2 is replaced by Ala (A); and/or Kabat residue 27 in LCDR1 is replaced by Gly (G); and/or Kabat residue 27B in LCDR1 is replaced by Ser (S); and/or Kabat residue 95 in LCDR3 is replaced by Pro (P).

According to a particular aspect of the invention there is provided an isolated binding member for human interleukin-4 receptor alpha (hIL-4Rα), wherein the HCDR1 has amino acid sequence SEQ ID NO: 363;
the HCDR2 has amino acid sequence SEQ ID NO: 364;
the HCDR3 has amino acid sequence SEQ ID NO: 365;
the LCDR1 has amino acid sequence SEQ ID NO: 368;
the LCDR2 has amino acid sequence SEQ ID NO: 369; and
the LCDR3 has amino acid sequence SEQ ID NO: 370.

According to another particular aspect of the invention there is provided an isolated binding member for human interleukin-4 receptor alpha (hIL-4Rα), wherein the HCDR1 has amino acid sequence SEQ ID NO: 233;
the HCDR2 has amino acid sequence SEQ ID NO: 234;
the HCDR3 has amino acid sequence SEQ ID NO: 235;
the LCDR1 has amino acid sequence SEQ ID NO: 238;
the LCDR2 has amino acid sequence SEQ ID NO: 239; and
the LCDR3 has amino acid sequence SEQ ID NO: 240;

In an antibody of the invention:
HCDR1 may be 5 amino acids long, consisting of Kabat residues 31-35;
HCDR2 may be 17 amino acids long, consisting of Kabat residues 50-65;
HCDR3 may be 9 amino acids long, consisting of Kabat residues 95-102;
LCDR1 may be 13 amino acids long, consisting of Kabat residues 24-34;
LCDR2 may be 7 amino acids long, consisting of Kabat residues 50-56; and/or,
LCDR3 may be 9 amino acids long, consisting of Kabat residues 89-97.
Kabat numbering of a set of HCDRs and LCDRs, wherein HCDR1 is Kabat residues 31-35, HCDR2 is Kabat residues 50-65, HCDR3 is Kabat residues 95-102 is shown in FIGS. 13 and 15; LCDR1 is Kabat residues 24-34, LCDR2 is Kabat residues 50-56 and LCDR3 is Kabat residues 89-97, is shown in FIGS. 14 and 16.

According to another aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, wherein the set of CDRs has 6 or fewer amino acid alterations from the reference set of CDRs present in the clone deposited at NCIMB on 9 Dec. 2008 with accession number: NCIMB 41600.

According to another aspect of the invention there is provided an isolated binding member for human interleukin-4 receptor alpha (hIL-4Rα), comprising a VH sequence as found in the clone deposited at NCIMB on 9 Dec. 2008 with accession number: NCIMB 41600.

According to another aspect of the invention there is provided an isolated binding member for human interleukin-4 receptor alpha (hIL-4Rα), comprising a VL sequence as found in the clone deposited at NCIMB on 9 Dec. 2008 with accession number: NCIMB 41600.

According to another aspect of the invention there is provided an isolated binding member for human interleukin-4 receptor alpha (hIL-4Rα), comprising a VH and VL sequence as found in the clone deposited at NCIMB on 9 Dec. 2008 with accession number: NCIMB 41600.

According to another aspect of the invention there is provided an isolated antibody or fragment of an antibody, wherein the antibody or the fragment immunospecifically binds to human interleukin-4 receptor alpha and comprises:
(a) a VH CDR1 having an amino acid sequence identical to or comprising 1, 2, or 3 amino acid residue substitutions relative to the VH CDR1 present in the clone deposited at NCIMB on 9 Dec. 2008 with accession number: NCIMB 41600;
(b) a VH CDR2 having an amino acid sequence identical to or comprising 1, 2, or 3 amino acid residue substitutions relative to the VH CDR2 present in the clone deposited at NCIMB on 9 Dec. 2008 with accession number: NCIMB 41600;
(c) a VH CDR3 having an amino acid sequence identical to or comprising 1, 2, or 3 amino acid residue substitutions relative to the VH CDR3 present in the clone deposited at NCIMB on 9 Dec. 2008 with accession number: NCIMB 41600;
(d) a VL CDR1 having an amino acid sequence identical to or comprising 1, 2, or 3 amino acid residue substitutions relative to the VL CDR1 present in the clone deposited at NCIMB on 9 Dec. 2008 with accession number: NCIMB 41600;
(e) a VL CDR2 having an amino acid sequence identical to or comprising 1, 2, or 3 amino acid residue substitutions relative to the VL CDR2 present in the clone deposited at NCIMB on 9 Dec. 2008 with accession number: NCIMB 41600; and
(f) a VL CDR3 having an amino acid sequence identical to or comprising 1, 2, or 3 amino acid residue substitutions relative to the VL CDR3 present in the clone deposited at NCIMB on 9 Dec. 2008 with accession number: NCIMB 41600.

According to another aspect of the invention there is provided an isolated antibody or fragment of an antibody, wherein the antibody or the fragment immunospecifically binds to human interleukin-4 receptor alpha and comprises:
(a) a VH sequence having an amino acid sequence identical to or comprising 1, 2, 3, 4, 5, or 6 amino acid residue substitutions relative to the VH sequence present in the clone deposited at NCIMB on 9 Dec. 2008 with accession number: NCIMB 41600;
(b) a VL sequence having an amino acid sequence identical to or comprising 1, 2, 3, 4, 5, or 6 amino acid residue substitutions relative to the VL sequence present in the clone deposited at NCIMB on 9 Dec. 2008 with accession number: NCIMB 41600.

An antibody may comprise an antibody molecule having one or more CDRs, e.g. a set of CDRs, within an antibody framework. For example, one or more CDRs or a set of CDRs of an antibody may be grafted into a framework (e.g. human framework) to provide an antibody molecule. Framework regions may comprise human germline gene segment sequences. Thus, the framework may be germlined, whereby one or more residues within the framework are changed to match the residues at the equivalent position in the most similar human germline framework. The skilled person can select a germline segment that is closest in sequence to the framework sequence of the antibody before germlining and test the affinity or activity of the antibodies to confirm that germlining does not significantly reduce antigen binding or potency in assays described herein. Human germline gene segment sequences are known to those skilled in the art and can be accessed for example from the VBase compilation (see Tomlinson. Journal of Molecular Biology. 224. 487-499, 1997).

In one embodiment, an antibody of the invention is an isolated human antibody molecule having a VH domain comprising a set of HCDRs in a human germline framework, e.g. Vh1_DP-7_(1-46). Thus, the VH domain framework regions FR1, FR2 and/or FR3 may comprise framework regions of human germline gene segment Vh1_DP-7_(1-46). FR4 may comprise a framework region of human germline j segment JH1, JH4 or JH5 (these j segments have identical amino acid sequences) or it may comprise a framework region of human germline j segment JH3. The amino acid sequence of VH FR1 may be SEQ ID NO: 442 (residues 1-30). The amino acid sequence of VH FR2 may be SEQ ID NO: 442 (residues 36-49). The amino acid sequence of VH FR3 may be SEQ ID NO: 442 (residues 66-94). The amino acid sequence of VH FR4 may be SEQ ID NO: 442 (103-113). Normally the binding member also has a VL domain comprising a set of LCDRs, e.g. in a human germline framework, e.g. Vλ1_DPL5. Thus, the VL domain framework regions FR1, FR2 and/or FR3 may comprise framework regions of human germline gene segment Vλ1_DPL5. FR4 may comprise a framework region of human germline j segment JL2 or JL3 (these j segments have identical amino acid sequences). The amino acid sequence of VL FR1 may be SEQ ID NO: 447 (residues 1-23). The amino acid sequence of VL FR2 may be SEQ ID NO: 447 (residues 35-49). The amino acid sequence of VL FR3 may be SEQ ID NO: 447 (residues 57-88). The amino acid sequence of VL FR4 may be SEQ ID NO: 447 (residues 98-107). A germlined VH or VL domain may or may not be germlined at one or more Vernier residues, but is normally not.

An antibody molecule or VH domain of the invention may comprise the following set of heavy chain framework regions:
FR1 SEQ ID NO: 442 (residues 1-30);
FR2 SEQ ID NO: 442 (residues 36-49);
FR3 SEQ ID NO: 442 (residues 66-94);
FR4 SEQ ID NO: 442 (residues 103-113);
or may comprise the said set of heavy chain framework regions with one, two, three, four, five, or six amino acid alterations, e.g. substitutions.

An antibody molecule or VL domain of the invention may comprise the following set of light chain framework regions:
FR1 SEQ ID NO: 447 (residues 1-23);
FR2 SEQ ID NO: 447 (residues 35-49);
FR3 SEQ ID NO: 447 (residues 57-88);
FR4 SEQ ID NO: 447 (residues 98-107);
or may comprise the said set of heavy chain framework regions with one, two, three, four, five, or six amino acid alterations, e.g. substitutions.

An amino acid alteration may be a substitution, an insertion (addition) or a deletion. The most common alteration is likely to be a substitution. For example, an antibody molecule of the invention may comprise a set of heavy and light chain framework regions, wherein:
heavy chain FR1 is SEQ ID NO: 192(residues 1-30);
heavy chain FR2 is SEQ ID NO: 192 (residues 36-49);
heavy chain FR3 is SEQ ID NO: 192 (residues 66-94);
heavy chain FR4 is SEQ ID NO: 192 (residues 103-113);
light chain FR1 is SEQ ID NO: 197 (residues 1-23);
light chain FR2 is SEQ ID NO: 197 (residues 35-49);
light chain FR3 is SEQ ID NO: 197 (residues 57-88);
light chain FR4 is SEQ ID NO: 197 (residues 98-107); or
may comprise the said set of heavy and light chain framework regions with seven or fewer, e.g. six or fewer, amino acid alterations, e.g. substitutions. For example there may be one or two amino acid substitutions in the set of heavy and light chain framework regions.

Antibodies 21-42 are based on Antibody 20, but with certain additional alterations within the CDRs and framework regions Like Antibody 20, Antibodies 21-42 bind hIL-4Rα and cyIL-4Rα. Such CDR and/or framework substitutions may therefore be considered as optional or additional substitutions generating antibodies with potentially greater binding.

Thus, in addition to the substitutions within any of the 6 CDR regions of the VH and VL domains, the antibodies may also comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3.

Suitable framework substitutions are shown in FIGS. 13 to 16. And an antibody of the present invention may comprise one or more of the specific substitutions shown in FIGS. 13 to 16.

An antibody molecule or VH domain of the invention may comprise a VH FR1 wherein Kabat residue 11 is Val or Glu and/or Kabat residue 12 is Lys or Arg; An antibody molecule or VH domain of the invention may comprise a VH FR2 wherein Kabat residue 37 is Ala or Val and/or Kabat residue 48 is Met or Val; An antibody molecule or VH domain of the invention may comprise a VH FR3 wherein Kabat residue 68 is Ser, Ala or Thr and/or Kabat residue 84 is Ser or Pro and/or Kabat residue 85 is Glu or Gly; An antibody molecule or VH domain of the invention may comprise a VH FR4 wherein Kabat residue 105 is Lys or Asn and/or Kabat residue 108 is Gln, Arg or Leu and/or Kabat residue 113 is Ser or Gly.

An antibody molecule or VL domain of the invention may comprise a VL FR1 wherein Kabat residue 1 is Gln or Leu and/or Kabat residue 2 is Ser or Pro or Ala and/or Kabat residue 3 is Val or Ala and/or Kabat residue 9 is Ser or Leu; An antibody molecule or VL domain of the invention may comprise a VL FR2 wherein Kabat residue 38 is Gln or Arg and/or Kabat residue 42 is Thr or Ala; An antibody molecule or VL domain of the invention may comprise a VL FR3 wherein Kabat residue 58 is Be or Val and/or Kabat residue 65 is Ser or Phe and/or Kabat residue 66 is Lys or Arg and/or Kabat residue 70 is Ser or Thr and/or Kabat residue 74 is Ala or Gly and/or Kabat residue 85 is Asp or Val and/or Kabat residue 87 is Tyr or Phe.

A non-germlined antibody has the same CDRs, but different frameworks, compared with a germlined antibody. Of the antibody sequences shown herein, VH and VL domains of Antibodies 24PGL and 37GL are germlined.

According to a further aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, which binding member has at least 73% amino acid sequence identity with the composite sequence of HCDR1, HCDR2, HCDR3, LCDR1, LCDR2 and LCDR3 in line sequence without any intervening framework sequences, of any of Antibodies 1-42. In a particular embodiment the isolated binding member has at least 78% amino acid sequence identity with the composite score of any of Antibodies 1-42.

According to a further aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, which binding member has at least 75% amino acid sequence identity with the composite sequence of HCDR1, HCDR2 and HCDR3 of any of Antibodies 1-42.

According to a further aspect of the invention there is provided an isolated binding member for human interleukin-4 receptor alpha (hIL-4Rα), comprising a set of CDRs: HCDR1, HCDR2, HCDR3, LCDR1, LCDR2 and LCDR3, which binding member has at least 65% amino acid sequence identity with the composite sequence of LCDR1, LCDR2 and LCDR3 of any of Antibodies 1-42.

An antibody of the present invention may be one which competes for binding to IL-4Rα with any binding member which both binds IL-4Rα and comprises an antibody, VH and/or VL domain, CDR e.g. HCDR3, and/or set of CDRs disclosed herein. Competition between antibodies may be assayed easily in vitro, for example using ELISA and/or by tagging a specific reporter molecule to one binding member which can be detected in the presence of one or more other untagged antibodies, to enable identification of antibodies which bind the same epitope or an overlapping epitope. Competition may be determined for example using ELISA in which IL-4Rα is immobilized to a plate and a first tagged binding member along with one or more other untagged antibodies is added to the plate. Presence of an untagged binding member that competes with the tagged binding member is observed by a decrease in the signal emitted by the tagged binding member. Such methods are readily known to one of ordinary skill in the art, and are described in more detail herein. In one embodiment, competitive binding is assayed using an epitope competition assay as described herein. An antibody of the present invention may comprise a antibody antigen-binding site that competes with an antibody molecule, for example especially an antibody molecule comprising a VH and/or VL domain, CDR e.g. HCDR3 or set of CDRs of the parent antibody or any of Antibodies 2 to 42 for binding to IL-4Rα.

Aspects of the invention provide antibodies that compete for binding to IL-4Rα with any binding member defined herein, e.g. compete with the parent antibody or any of Antibodies 2 to 42, e.g. in scFv or IgG1, IgG2 or IgG4 format. An antibody that competes for binding to IL-4Rα with any binding member defined herein may have any one or more of the structural and/or functional properties disclosed herein for antibodies of the invention.

### Methods of Treatment

Antibodies according to the invention may be used in a method of treatment or diagnosis of the human or animal body, such as a method of treatment (which may include prophylactic treatment) of a disease or disorder in a human patient which comprises administering to said patient an effective amount of an antibody of the invention. Conditions treatable in accordance with the present invention include any in which IL-4Rα, IL-4 and/or IL-13 plays a role, as discussed in detail elsewhere herein.

These and other aspects of the invention are described in further detail below.

Antibodies of the present invention may be used in methods of diagnosis or treatment in human or animal subjects, e.g. human. For instance, antibodies may be used in diagnosis or treatment of IL-4Rα-associated diseases or disorders, examples of which are referred to elsewhere herein.

Particular conditions for which an antibody of the invention may be used in treatment or diagnosis include: asthma, COPD (including chronic bronchitis, small airway disease and emphysema), inflammatory bowel disease, fibrotic conditions (including systemic sclerosis, pulmonary fibrosis, parasite-induced liver fibrosis, and cystic fibrosis, allergy (including for example atopic dermatitis and food allergy), transplantation therapy to prevent transplant rejection, as well as suppression of delayed-type hypersensitivity or contact hypersensitivity reactions, as adjuvants to allergy immunotherapy and as vaccine adjuvants. In some embodiments, the present invention is directed to a method of treating an inflammatory skin disorder by administering an antibody formulation as described herein. In some embodiments, the inflammatory skin disorder is atopic dermatitis.

In certain aspects, this disclosure provides a method of treating a patient diagnosed with a pulmonary disease or disorder (e.g., asthma, idiopathic pulmonary disease (IPF) or COPD) or a chronic inflammatory skin disease or disorder (e.g., or atopic dermatitis) comprising administering the antibody formulations described herein. In some embodiments, the invention is directed to a method of treating a chronic inflammatory skin disease or disorder, comprising administering the antibody formulations described herein. In some embodiments, the chronic inflammatory skin disease is selected from the group consisting of atopic dermatitis, allergic contact dermatitis, eczema or psoriasis.

The term “Idiopathic Pulmonary Fibrosis” (IPF) refers to a disease characterized by progressive scarring, or fibrosis, of the lungs. It is a specific type of interstitial lung disease in which the alveoli gradually become replaced by fibrotic tissue. With IPF, progressive scarring causes the normally thin and pliable tissue to thicken and become stiff, making it more difficult for the lungs to expand, preventing oxygen from readily getting into the bloodstream. See, e.g., Am. J. Respir. Crit. Care Med. 2000. 161:646-664.

Atopic dermatitis is a common chronic inflammatory skin disease that is often associated with other atopic disorders such as allergic rhinitis and asthma (Bieber, New England Journal of Medicine, 2008, 358: 1483-1494). Upregulation of IL-13 mRNA has been observed in subacute and chronic lesions of atopic dermatitis (Tazawa et al., Arch. Dermatol. Res., 2004, 295:459-464; Purwar et al, J. Invest. Derm., 2006, 126, 1043-1051; Oh et al., J Immunol., 2011, 186:7232-42).

As used herein, the term “atopic dermatitis” refers to a chronic inflammatory, relapsing, non-contagious and itchy skin disorder that is often associated with other atopic disorders such as allergic rhinitis and asthma (Bieber, New England Journal of Medicine, 2008, 358: 1483-1494). The term “atopic dermatitis” is equivalent to “neurodermatitis”, “atopic eczema” or “endogenous eczema”. Particular forms of atopic dermatitis, which get their names from the place where they occur or from their appearance or from the stress factors which provoke them, are, according to the present disclosure also comprised by the term “atopic dermatitis”. These include, but are not limited to, eczema flexurarum, eczema mulluscatum, eczema verrucatum, eczema vaccinatum, eczema dyskoides, dyshydrotic eczema, microbial eczema, nummular eczema, seborrhobic eczema and other forms of eczema; perioral dermatitis and periorbital dermatitis. As used herein, the term atopic dermatitis also comprises the frequently occurring bacterial secondary infections such as those due to e.g. Staphylococcus aureus infections, pyodermas such as impetigo contagiosa and its derivatives as well as the follicularis barbae or viral secondary infections. IL-13 is involved in the pathogenesis of the disease and is an important in vivo inducer. See, e.g., Oh et al., J. Immunol. 186:7232-42 (2011); Tazawa et al., Arch. Dermatol. Res. 295:459-464 (2004); Metwally et al. Egypt J. Immunol. 11:171-7 (2004).

Thus, antibodies of the invention are useful as therapeutic agents in the treatment of conditions involving IL-4, IL-13 or IL-4Rα expression and/or activity. One embodiment, among others, is a method of treatment comprising administering an effective amount of an antibody of the invention to a patient in need thereof, wherein functional consequences of IL-4Rα activation are decreased. Another embodiment, among others, is a method of treatment comprising (i) identifying a patient demonstrating IL-4, IL-13 or IL-4Rα expression or activity, for instance using the diagnostic methods described above, and (ii) administering an effective amount of an antibody of the invention to the patient, wherein the functional consequences of IL-4Rα activation are attenuated. An effective amount according to the invention is an amount that modulates (e.g. decreases) the functional consequences of IL-4Rα activation so as to modulate (e.g. decrease or lessen) the severity of at least one symptom of the particular disease or disorder being treated, but not necessarily cure the disease or disorder. Accordingly, one embodiment of the invention is a method of treating or reducing the severity of at least one symptom of any of the disorders referred to herein, comprising administering to a patient in need thereof an effective amount of one or more antibodies of the present invention alone or in a combined therapeutic regimen with another appropriate medicament known in the art or described herein such that the severity of at least one symptom of any of the disorders is reduced. Another embodiment of the invention, among others, is a method of antagonizing at least one effect of IL-4Rα comprising contacting with or administering an effective amount of one or more antibodies of the present invention such that said at least one effect of IL-4Rα is antagonized, e.g. the ability of IL-4Rα to form a complex (the precursor to active signalling) with IL-4.

Accordingly, further aspects of the invention provide methods of treatment comprising administration of an antibody as provided, or pharmaceutical compositions comprising such an antibody, and/or use of such an antibody in the manufacture of a medicament for administration, for example in a method of making a medicament or pharmaceutical composition comprising formulating the binding member with a pharmaceutically acceptable excipient. A pharmaceutically acceptable excipient may be a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable vehicles are well known and will be adapted by the person skilled in the art as a function of the nature and of the mode of administration of the active compound(s) chosen.

### Antibody Formulations

Further aspects of the present invention provide for antibody formulations containing antibodies of the invention, and their use in methods of inhibiting and/or neutralizing IL-4Rα, including methods of treatment of the human or animal body by therapy.

In some embodiments, the antibody formulations are pharmaceutically acceptable. The term “pharmaceutically acceptable” refers to a compound or protein that can be administered to an animal (for example, a mammal) without significant adverse medical consequences.

In some embodiments, the antibody formulation comprises a physiologically acceptable carrier. The term “physiologically acceptable carrier” refers to a carrier which does not have a significant detrimental impact on the treated host and which retains the therapeutic properties of the compound with which it is administered. One exemplary physiologically acceptable carrier is physiological saline. Other physiologically acceptable carriers and their formulations are known to one skilled in the art and are described, for example, in Remington's Pharmaceutical Sciences, (18th edition), ed. A. Gennaro, 1990, Mack Publishing Company, Easton, Pa., incorporated herein by reference.

Antibodies of the present invention will usually be administered in the form of a pharmaceutical composition, which may comprise at least one component in addition to the antibody. Thus pharmaceutical compositions according to the present invention, and for use in accordance with the present invention, may comprise, in addition to antibody, one or more of a viscosity modifier, a non-ionic surfactant, a formulation buffer, a pharmaceutically acceptable excipient, carrier, buffer, stabilizer or other materials known to those skilled in the art. Such materials should be non-toxic and should not interfere with the efficacy of the antibody. The precise nature of the carrier or other material will depend on the route of administration, which may be oral, inhaled or by injection, e.g. intravenous. In one embodiment the composition is sterile.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be employed, as required, including buffers such as phosphate, citrate, histidine and other organic acids; antioxidants such as ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3′-pentanol; and m-cresol); low molecular weight polypeptides; proteins such as serum albumin, gelatin or immunoglobulins; hydrophilic polymers such as polyvinylpyrrolidone; amino acids such as glycine, glutamine, asparagines, histidine, arginine, or lysine; monosaccharides, disaccharides and other carbohydrates including glucose, mannose or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Antibodies of the present invention may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Formulations of anti-IL-4Rα will depend upon the intended route of delivery: for example, formulations for pulmonary delivery may consist of particles with physical properties that ensure penetration into the deep lung upon inhalation; topical formulations may include viscosity modifying agents, which prolong the time that the drug is resident at the site of action. In certain embodiments, the binding member may be prepared with a carrier that will protect the binding member against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known to those skilled in the art. See, e.g., Robinson, 1978.

Anti-IL-4Rα treatment with an antibody of the invention may be given orally (for example nanobodies) by injection (for example, subcutaneously, intra-articular, intravenously, intraperitoneal, intra-arterial or intramuscularly), by inhalation, by the intravesicular route (instillation into the urinary bladder), or topically (for example intraocular, intranasal, rectal, into wounds, on skin). The treatment may be administered by pulse infusion, particularly with declining doses of the binding member. The route of administration can be determined by the physicochemical characteristics of the treatment, by special considerations for the disease or by the requirement to optimize efficacy or to minimize side-effects. One particular route of administration is intravenous. Another route of administering pharmaceutical compositions of the present invention is subcutaneously. It is envisaged that anti-IL-4Rα treatment will not be restricted to use in hospitals or doctor's offices but rather may include homes and places of work. Therefore, subcutaneous injection using a needle-free device is advantageous.

In some embodiments of the invention, the antibody formulation contains a high concentration of antibody. In some embodiments, the antibody concentration in the antibody formulation is greater than 100 mg/mL of antibody. In some embodiments, the antibody concentration is about 100 mg/mL to about 200 mg/mL, about 120 mg/mL to about 180 mg/mL, about 140 mg/mL to about 160 mg/mL, or about 150 mg/mL.

In some embodiments, the antibody formulation contains a lower concentration of antibody, e.g., about 10 mg/mL to about 100 mg/mL. In some embodiments, the antibody concentration in the antibody formation is about 20 mg/mL to about 80 mg/mL, about 30 mg/mL to about 70 mg/mL, about 40 mg/mL to about 60 mg/mL, or about 50 mg/mL In some embodiments, antibody formulations comprising the lower concentrations of antibodies further comprise an excipient. The term excipient refers to a pharmacologically inactive substance formulated with the antibody as described herein. In some embodiments, the excipient can assist in the prevention of denaturation or otherwise assist in stabilizing the antibody at lower concentrations.

Suitable excipients that may be used in the pharmaceutical compositions are known in the art. Examples can be taken e.g. from the handbook: Gennaro, Alfonso R.: “Remington's Pharmaceutical Sciences”, Mack Publishing Company, Easton, Pa., 1990. In some embodiments, the excipient is an “uncharged” excipient, i.e., the excipient does not carry either a positive “+” or negative “−” charge. In some embodiments, the excipient is selected from the group consisting of fructose, glucose, mannose, sorbose, xylose, lactose, maltose, sucrose, dextran, pullulan, dextrin, cyclodextrins, soluble starch, trehalose, sorbitol, erythritol, isomalt, lactitol, maltitol, xylitol, glycerol, lactitol, hydroxyethyl starch, water-soluble glucans. In some embodiments, the excipient is trehalose.

In some embodiments, the trehalose is about 50 mM to about 800 mM, about 100 mM to about 500 mM, about 150 mM to about 400 mM, about 200 mM, about 400 mM, about 200 mM, about 300 mM, or about 250 mM in the antibody formulation, e.g., an antibody formulation comprising 20 to 100 mg/mL antibody. In one embodiment, the trehalose is about 250 mM in the antibody formulation.

In some embodiments, the formulation buffer is essentially free of phosphate. The term “essentially free of phosphate” when referring to a formulation buffer refers to a buffer system wherein the phosphate ion is not used to buffer the pH. Thus, a buffer essentially free of phosphate could have the phosphate moiety present (i.e., covalently bonded) on a compound at the working pH, but no phosphate ions would be present. In some embodiments, the antibody formulation is essentially free of phosphate. The term “essentially free of phosphate” when referring to an antibody formulation refers to a buffer system wherein the phosphate ion is not used to buffer the pH in the antibody formulation.

In some embodiments, the antibody formulation comprises a viscosity modifier. In some instances, the antibody formulation has high viscosity due to the high concentration of antibody. Various viscosity modifiers are known to those in the art. In some embodiments, the viscosity modifier is selected from the group consisting of histidine, arginine, lysine, polyvinyl alcohol, polyalkyl cellulose, hydroxyalkyl cellulose, glycerin, polyethylene glycol, glucose, dextrose, and sucrose. In some embodiments, the viscosity modifier is lysine, arginine, or histidine. In some embodiments, the viscosity modifier is arginine. In some embodiments, the viscosity modifier comprises a salt form, for example a salt of arginine, lysine or histidine. In some embodiments, the viscosity modifier is an amino acid, e.g., an L-form amino acid such as L-arginine, L-lysine, or L-histidine. In some embodiments, the viscosity modifier is in a concentration of about 50 mM to about 400 mM, or about 100 mM to about 250 mM. In some embodiments, the viscosity modifier is in a concentration of about 190 mM. In some embodiments, the viscosity modifier is arginine in a concentration of about 100 mM to about 250 mM. In some embodiments, the viscosity modifier is arginine-HCl in a concentration of about 100 mM to about 250 mM. In some embodiments, the viscosity modifier is arginine in a concentration of about 190 mM. In some embodiments, the viscosity modifier is arginine-HCl in a concentration of about 190 mM.

In some embodiments, viscosity modifier is added in an amount to obtain a viscosity of less than about 40 cP at 23° C., less than about 30 cP at 23° C., less than about 25 cP at 23° C., or less than about 20 cP at 23° C., In some embodiments, viscosity modifier is added in an amount to obtain a viscosity of about 1 cP to about 40 cP at 23° C., about 2 cP to about 30 cP at 23° C., about 5 cP to about 25 cP at 23° C., or about 10 cP to about 20 cP at 23° C.

In some embodiments, a surfactant is present in the antibody formation. Various surfactants are known to those in the art. In some embodiments, the surfactant is a non-ionic surfactant. In some embodiments, the non-ionic surfactant is selected from the group consisting of Triton X-100, Tween 80, polysorbate 20, polysorbate 80, nonoxynol-9, polyoxamer, stearyl alcohol, or sorbitan monostearate. In some embodiments, the non-ionic surfactant is polysorbate 80. The inventors have found that when formulating an IL-4Rα antibody, in some embodiments, the formulation comprises about 0.002% to about 0.4%, 0.005% to about 0.15%, about 0.002% to about 0.2%, about 0.01% to about 0.1%, or about 0.02% to about 0.08% of a non-ionic surfactant. In some embodiments, formulation comprises about 0.04% of a non-ionic surfactant. In some embodiments, the formulation comprises about 0.002% to about 0.4%, about 0.002% to about 0.2%, about 0.005% to about 0.15%, about 0.01% to about 0.1%, or about 0.02% to about 0.08% of a polysorbate 80. In some embodiments, formulation comprises about 0.04% of a polysorbate 80. In some embodiments, the non-ionic surfactant is at or above the CMC value, up to 0.5%. In some embodiments, the concentration of the non-ionic surfactant is sufficient to prevent or inhibit aggregation. In some embodiments, aggregration is determined by visual analysis.

In some embodiments, the antibody formulation comprises a formulation buffer. In some embodiments, the formulation buffer is an acetate buffer, TRIS buffer, HEPES buffer, hydrochloride buffer, arginine buffer, glycine buffer, citrate buffer, or TES buffer. In some embodiments, the formulation buffer is an arginine buffer. In some embodiments, the arginine buffer comprises arginine hydrochloride. In some embodiments, the arginine buffer further comprises histidine. In some embodiments, the histidine is L-histidine/L-histidine hydrochloride.

The formulation buffer can comprise various concentrations of arginine. In some embodiments, the formulation buffer comprises about 10 mM to about 40 mM L-histidine/L-histidine hydrochloride. In some embodiments, the formulation buffer comprises about 25 mM L-histidine/L-histidine hydrochloride.

Various additional excipients can be found in the antibody formulation. In some embodiments, the formulation further comprises a salt, e.g., a NaCl, or KCl salt. In some embodiments, the salt is about 100 mM to about 200 mM NaCl.

The antibody formulation can have various pH levels. In some embodiments, the formulation has a pH of about 5 to about 8, about 5.5 to about 8, about 6 to about 8, about 6.5 to about 8, about 7 to about 8. In some embodiments, the formulation has a pH of about 7.0, about 7.1, about 7.2, about 7.3, about 7.4, about 7.5, about 7.6, about 7.7, about 7.8 or about 7.9. In some embodiments, the formulation has a pH of about 7.2 to about 7.6, or about 7.4. In some embodiments, the formulation has a pH of about 5.5 to about 6.5. In some embodiments, the formulation has a pH of about 5.6, about 5.7, about 5.8, about 5.9, about 6.0, about 6.1, about 6.2, about 6.3, or about 6.4. In some embodiments, the formulation has a pH of about 6.0.

In some embodiments, the antibody formulation is a liquid formulation, suitable for a subcutaneous administration. In some embodiments, the antibody formulation is a lyophilized formulation. In some embodiments, the lyophilized formulation is reconstituted to a liquid (e.g., aqueous) form prior to administration. In some embodiments, the antibody has not been subjected to lyophilization.

The antibody formulation of the invention may be suitable for storage for extended periods of time. In some embodiments, the formulation is stable upon storage at about 40° C. for at least about 1 week, at least about 2 week, at least about 3 weeks, at least about 1 month, at least about 2 months, at least about 3 months, at least about 6 months, at least about 1 year, or at least about 18 months. In some embodiments, the antibody formulation is stable upon storage at about 40° C. for about 2 weeks to about 1 year, about 1 month to about 1 year, about 2 months to about 1 year, or about 3 months to about 1 year. In some embodiments, the antibody formulation is stable upon storage at about 40° C. for about 2 weeks to about 6 months, about 1 month to about 6 months, about 2 months to about 6 months, or about 3 months to about 6 months.

In some embodiments, the antibody formulation described herein has reduced particle formation during agitation. Particle formulation analysis is described herein in Example 5. In some embodiments, the antibody formulation has less than 1,000 “≧10 μm particles”/mL when exposed to the agitation experiment of Example 5. In some embodiments, the antibody formulation has less than 500 “≧10 μm particles”/mL when exposed to the agitation experiment of Example 5. In some embodiments, the antibody formulation has less than 100 “≧10 μm particles”/mL when exposed to the agitation experiment of Example 5. In some embodiments, the antibody formulation has less than 1,000 “≧10 μm particles”/mL when exposed to the agitation experiment of Example 5. In some embodiments, the antibody formulation has less than 500 “≧10 μm particles”/mL when exposed to the agitation experiment of Example 5. In some embodiments, the antibody formulation has less than 100 “≧10 μm particles”/mL when exposed to the agitation experiment of Example 5.

In some embodiments, the formulation is stable upon storage at about 25° C. for at least 3 months, at least 6 months, at least 9 months, or at least 1 year. In some embodiments, the formulation is stable upon storage at about 5° C. for at least 18 months, at least 24 months, or at least 36 months.

In some embodiments, the antibody stored at about 40° C. for at least 1 month retains at least 50%, at least 60%, at least 70%, at least 80%, at least 90%, or at least 95% of binding ability to an hIL-4Rα polypeptide compared to a reference antibody which has not been stored. In some embodiments, the antibody stored at about 5° C. for at least 6 months retains at least 50%, at least 60%, at least 70%, at least 80%, at least 90%, or at least 95% of binding ability to an hIL-4Rα polypeptide compared to a reference antibody which has not been stored. In some embodiments, the antibody stored at about 40° C. for at least 1 month retains at least 50% or at least 95% of binding ability to an hIL-4Rα polypeptide compared to a reference antibody which has not been stored. In some embodiments, the antibody stored at about 5° C. for at least 6 months retains at least 50% or at least 95% of binding ability to an hIL-4Rα polypeptide compared to a reference antibody which has not been stored.

In some embodiments, the formulation is an injectable formulation. In some embodiments, the formulation is suitable for intravenous, subcutaneous, or intramuscular administration.

The antibody formulation of the present invention can be placed in a sealed container for transport, storage and/or administration. In some embodiments, the sealed container is a sealed vial or a sealed syringe. In some embodiments, the container is a pre-filled syringe. In some embodiments, the container is a single use container which contains one dosage of the antibody. In some embodiments, the invention is directed to a pharmaceutical unit dosage form suitable for parenteral administration to a human which comprises the antibody formulation in a suitable container.

The invention can also be directed to a kit comprising the antibody formulation described herein, the container described herein, the unit dosage form described herein, and/or the pre-filled syringe described herein.

A composition may be administered alone or in combination with other treatments, concurrently or sequentially or as a combined preparation with another therapeutic agent or agents, dependent upon the condition to be treated.

An antibody for IL-4Rα may be used as part of a combination therapy in conjunction with an additional medicinal component. Combination treatments may be used to provide significant synergistic effects, particularly the combination of an anti-IL-4Rα binding member with one or more other drugs. An antibody for IL-4Rα may be administered concurrently or sequentially or as a combined preparation with another therapeutic agent or agents, for the treatment of one or more of the conditions listed herein.

In some embodiments, the antibody composition of the present invention may comprise the antibody described herein in combination or addition with one or more of the following agents:
a cytokine or agonist or antagonist of cytokine function (e.g. an agent which acts on cytokine signalling pathways, such as a modulator of the SOCS system), such as an alpha-, beta- and/or gamma-interferon; insulin-like growth factor type I (IGF-1), its receptors and associated binding proteins; interleukins (IL), e.g. one or more of IL-1 to -33, and/or an interleukin antagonist or inhibitor, such as anakinra; inhibitors of receptors of interleukin family members or inhibitors of specific subunits of such receptors, a tumour necrosis factor alpha (TNF-α) inhibitor, such as an anti-TNF monoclonal antibodies (for example infliximab, adalimumab and/or CDP-870) and/or a TNF receptor antagonist, e.g. an immunoglobulin molecule (such as etanercept) and/or a low-molecular-weight agent, such as pentoxyfylline;
a modulator of B cells, e.g. a monoclonal antibody targeting B-lymphocytes (such as CD20 (rituximab) or MRA-aIL16R) or T-lymphocytes (e.g. CTLA4-Ig or Abatacept);
a modulator that inhibits osteoclast activity, for example an antibody to RANKL;
a modulator of chemokine or chemokine receptor function, such as an antagonist of CCR1, CCR2, CCR2A, CCR2B, CCR3, CCR4, CCR5, CCR6, CCR7, CCR8, CCR9, CCR10 or CCR11 (for the C-C family); CXCR1, CXCR2, CXCR3, CXCR4, CXCR5, CXCR6 or CXCL13 (for the C-X-C family) or CX₃CR1 (for the C-X₃-C family);
an inhibitor of matrix metalloproteases (MMPs), i.e. one or more of the stromelysins, the collagenases and the gelatinases as well as aggrecanase, especially collagenase-1 (MMP-1), collagenase-2 (MMP-8), collagenase-3 (MMP-13), stromelysin-1 (MMP-3), stromelysin-2 (MMP-10) and/or stromelysin-3 (MMP-11) and/or MMP-9 and/or MMP-12, e.g. an agent such as doxycycline;
a leukotriene biosynthesis inhibitor, 5-lipoxygenase (5-LO) inhibitor or 5-lipoxygenase activating protein (FLAP) antagonist, such as zileuton; ABT-761; fenleuton; tepoxalin; Abbott-79175; Abbott-85761; N-(5-substituted)-thiophene-2-alkylsulfonamides; 2,6-di-tert-butylphenolhydrazones; methoxytetrahydropyrans such as Zeneca ZD-2138; the compound SB-210661; a pyridinyl-substituted 2-cyanonaphthalene compound, such as L-739,010; a 2-cyanoquinoline compound, such as L-746,530; indole and/or a quinoline compound, such as MK-591, MK-886 and/or BAY x 1005;
a receptor antagonist for leukotrienes (LT) B4, LTC4, LTD4, and LTE4, selected from the group consisting of the phenothiazin-3-1s, such as L-651,392; amidino compounds, such as CGS-25019c; benzoxalamines, such as ontazolast; benzenecarboximidamides, such as BIIL 284/260; and compounds, such as zafirlukast, ablukast, montelukast, pranlukast, verlukast (MK-679), RG-12525, Ro-245913, iralukast (CGP 45715A) and BAY x 7195;
a phosphodiesterase (PDE) inhibitor, such as a methylxanthanine, e.g. theophylline and/or aminophylline; and/or a selective PDE isoenzyme inhibitor, e.g. a PDE4 inhibitor and/or inhibitor of the isoform PDE4D and/or an inhibitor of PDE5;
a histamine type 1 receptor antagonist, such as cetirizine, loratadine, desloratadine, fexofenadine, acrivastine, terfenadine, astemizole, azelastine, levocabastine, chlorpheniramine, promethazine, cyclizine, and/or mizolastine (generally applied orally, topically or parenterally);
a proton pump inhibitor (such as omeprazole) or gastroprotective histamine type 2 receptor antagonist;
an antagonist of the histamine type 4 receptor;
an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride and ethylnorepinephrine hydrochloride;
an anticholinergic agent, e.g. a muscarinic receptor (e.g. M1, M2, M3, M4 or M5) antagonist, such as atropine, hyoscine, glycopyrrrolate, ipratropium bromide, tiotropium bromide, oxitropium bromide, pirenzepine and telenzepine;
a beta-adrenoceptor agonist (including beta receptor subtypes 1-4), such as isoprenaline, salbutamol, formoterol, salmeterol, terbutaline, orciprenaline, bitolterol mesylate and/or pirbuterol, e.g. a chiral enantiomer thereof,
a chromone, e.g. sodium cromoglycate and/or nedocromil sodium;
a glucocorticoid, such as flunisolide, triamcinolone acetonide, beclomethasone dipropionate, budesonide, fluticasone propionate, ciclesonide, and/or mometasone furoate;
an agent that modulate nuclear hormone receptors, such as a PPAR;
an immunoglobulin (Ig) or Ig preparation or an antagonist or antibody modulating Ig function, such as anti-IgE (e.g. omalizumab);
other systemic or topically-applied anti-inflammatory agent, e.g. thalidomide or a derivative thereof, a retinoid, dithranol and/or calcipotriol;
combinations of aminosalicylates and sulfapyridine, such as sulfasalazine, mesalazine, balsalazide, and olsalazine; and immunomodulatory agents, such as the thiopurines; and corticosteroids, such as budesonide;
an antibacterial agent, e.g. a penicillin derivative, a tetracycline, a macrolide, a beta-lactam, a fluoroquinolone, metronidazole and/or an inhaled aminoglycoside; and/or an antiviral agent, e.g. acyclovir, famciclovir, valaciclovir, ganciclovir, cidofovir; amantadine, rimantadine; ribavirin; zanamavir and/or oseltamavir; a protease inhibitor, such as indinavir, nelfinavir, ritonavir and/or saquinavir; a nucleoside reverse transcriptase inhibitor, such as didanosine, lamivudine, stavudine, zalcitabine, zidovudine; a non-nucleoside reverse transcriptase inhibitor, such as nevirapine, efavirenz;
a cardiovascular agent, such as a calcium channel blocker, beta-adrenoceptor blocker, angiotensin-converting enzyme (ACE) inhibitor, angiotensin-2 receptor antagonist; lipid lowering agent, such as a statin and/or fibrate; a modulator of blood cell morphology, such as pentoxyfylline; a thrombolytic and/or an anticoagulant, e.g. a platelet aggregation inhibitor;
a CNS agent, such as an antidepressant (such as sertraline), anti-Parkinsonian drug (such as deprenyl, L-dopa, ropinirole, pramipexole; MAOB inhibitor, such as selegine and rasagiline; comP inhibitor, such as tasmar; A-2 inhibitor, dopamine reuptake inhibitor, NMDA antagonist, nicotine agonist, dopamine agonist and/or inhibitor of neuronal nitric oxide synthase) and an anti-Alzheimer's drug, such as donepezil, rivastigmine, tacrine, COX-2 inhibitor, propentofylline or metrifonate;
an agent for the treatment of acute and chronic pain, e.g. a centrally or peripherally-acting analgesic, such as an opioid analogue or derivative, carbamazepine, phenyloin, sodium valproate, amitryptiline or other antidepressant agent, paracetamol, or non-steroidal anti-inflammatory agent;
a parenterally or topically-applied (including inhaled) local anaesthetic agent, such as lignocaine or an analogue thereof;
an anti-osteoporosis agent, e.g. a hormonal agent, such as raloxifene, or a biphosphonate, such as alendronate;
(i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitors including VLA-4 antagonist; (vi) a cathepsin; (vii) a kinase inhibitor, e.g. an inhibitor of tyrosine kinases (such as Btk, Itk, Jak3 MAP examples of inhibitors might include Gefitinib, Imatinib mesylate), a serine/threonine kinase (e.g. an inhibitor of MAP kinase, such as p38, JNK, protein kinases A, B and C and IKK), or a kinase involved in cell cycle regulation (e.g. a cylin dependent kinase); (viii) a glucose-6 phosphate dehydrogenase inhibitor; (ix) a kinin-B.sub1.- and/or B.sub2.-receptor antagonist; (x) an anti-gout agent, e.g. colchicine; (xi) a xanthine oxidase inhibitor, e.g. allopurinol; (xii) a uricosuric agent, e.g. probenecid, sulfinpyrazone, and/or benzbromarone; (xiii) a growth hormone secretagogue; (xiv) transforming growth factor (TGFβ); (xv) platelet-derived growth factor (PDGF); (xvi) fibroblast growth factor, e.g. basic fibroblast growth factor (bFGF); (xvii) granulocyte macrophage colony stimulating factor (GM-CSF); (xviii) capsaicin cream; (xix) a tachykinin NK.sub 1. and/or NK.sub3. receptor antagonist, such as NKP-608C, SB-233412 (talnetant) and/or D-4418; (xx) an elastase inhibitor, e.g. UT-77 and/or ZD-0892; (xxi) a TNF-alpha converting enzyme inhibitor (TACE); (xxii) induced nitric oxide synthase (iNOS) inhibitor or (xxiii) a chemoattractant receptor-homologous molecule expressed on TH2 cells (such as a CRTH2 antagonist); (xxiv) an inhibitor of a P38 (xxv) agent modulating the function of Toll-like receptors (TLR) and (xxvi) an agent modulating the activity of purinergic receptors, such as P2×7; (xxvii) an inhibitor of transcription factor activation, such as NFkB, API, and/or STATS.

An inhibitor may be specific or may be a mixed inhibitor, e.g. an inhibitor targeting more than one of the molecules (e.g. receptors) or molecular classes mentioned above.

The binding member could also be used in association with a chemotherapeutic agent or another tyrosine kinase inhibitor in co-administration or in the form of an immunoconjugate. Fragments of said antibody could also be use in bispecific antibodies obtained by recombinant mechanisms or biochemical coupling and then associating the specificity of the above described antibody with the specificity of other antibodies able to recognize other molecules involved in the activity for which IL-4Rα is associated.

For treatment of an inflammatory disease, e.g. rheumatoid arthritis, osteoarthritis, asthma, allergic rhinitis, chronic obstructive pulmonary disease (COPD), inflammatory skin disease such as atopic dermatitis, or psoriasis, an antibody of the invention may be combined with one or more agents, such as non-steroidal anti-inflammatory agents (hereinafter NSAIDs) including non-selective cyclo-oxygenase (COX)-1/COX-2 inhibitors whether applied topically or systemically, such as piroxicam, diclofenac, propionic acids, such as naproxen, flurbiprofen, fenoprofen, ketoprofen and ibuprofen, fenamates, such as mefenamic acid, indomethacin, sulindac, azapropazone, pyrazolones, such as phenylbutazone, salicylates, such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intra-muscular, intra-venous or intra-articular routes); methotrexate, leflunomide; hydroxychloroquine, d-penicillamine, auranofin or other parenteral or oral gold preparations; analgesics; diacerein; intra-articular therapies, such as hyaluronic acid derivatives; and nutritional supplements, such as glucosamine.

An antibody of the invention can also be used in combination with an existing therapeutic agent for the treatment of cancer. Suitable agents to be used in combination include:

(i) antiproliferative/antineoplastic drugs and combinations thereof, as used in medical oncology, such as Gleevec (imatinib mesylate), alkylating agents (for example cis-platin, carboplatin, cyclophosphamide, nitrogen mustard, melphalan, chlorambucil, busulphan and nitrosoureas); antimetabolites (for example antifolates, such as fluoropyrimidines like 5-fluorouracil and tegafur, raltitrexed, methotrexate, cytosine arabinoside, hydroxyurea, gemcitabine and paclitaxel); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecins);

(ii) cytostatic agents, such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase, such as finasteride;

(iii) Agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors, such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents, such as those which inhibit the effects of vascular endothelial growth factor (for example the anti-vascular endothelial cell growth factor antibody bevacizumab, compounds, such as those disclosed in International Patent Applications WO 97/22596, WO 97/30035, WO 97/32856 and WO 98/13354, each of which is incorporated herein in its entirety) and compounds that work by other mechanisms (for example linomide, inhibitors of integrin αvβ3 function and angiostatin);

(vi) vascular damaging agents, such as combretastatin A4 and compounds disclosed in International Patent Applications WO 99/02166, WO 00/40529, WO 00/41669, WO 01/92224, WO 02/04434 and WO 02/08213 (each of which is incorporated herein in its entirety);

(vii) antisense therapies, for example those which are directed to the targets listed above, such as ISIS 2503, an anti-ras antisense;

(viii) gene therapy approaches, including for example approaches to replace aberrant genes, such as aberrant p53 or aberrant BRCA1 or BRCA2, GDEPT (gene directed enzyme pro-drug therapy) approaches, such as those using cytosine deaminase, thymidine kinase or a bacterial nitroreductase enzyme and approaches to increase patient tolerance to chemotherapy or radiotherapy, such as multi-drug resistance gene therapy; and

(ix) immunotherapeutic approaches, including for example ex vivo and in vivo approaches to increase the immunogenicity of patient tumour cells, such as transfection with cytokines, such as interleukin 2, interleukin 4 or granulocyte macrophage colony stimulating factor, approaches to decrease T-cell anergy, approaches using transfected immune cells, such as cytokine-transfected dendritic cells, approaches using cytokine-transfected tumour cell lines and approaches using anti-idiotypic antibodies.

An antibody of the invention and one or more of the above additional medicinal components may be used in the manufacture of a medicament. The medicament may be for separate or combined administration to an individual, and accordingly may comprise the binding member and the additional component as a combined preparation or as separate preparations. Separate preparations may be used to facilitate separate and sequential or simultaneous administration, and allow administration of the components by different routes e.g. oral and parenteral administration.

In accordance with the present invention, compositions provided may be administered to mammals. Administration may be in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the type of binding member, the method of administration, the scheduling of administration and other factors known to medical practitioners. Prescription of treatment, e.g. decisions on dosage etc, is within the responsibility of general practitioners and other medical doctors, and may depend on the severity of the symptoms and/or progression of a disease being treated. Appropriate doses of antibody are well known in the art (Ledermann et al. Int. J. Cancer 47:659-664, 1991; Bagshawe et al. Antibody, Immunoconjugates and Radiopharmaceuticals 4:915-922, 1991). Specific dosages indicated herein, or in the Physician's Desk Reference (2003) as appropriate for the type of medicament being administered, may be used. A therapeutically effective amount or suitable dose of an antibody of the invention can be determined by comparing it's in vitro activity and in vivo activity in an animal model. Methods for extrapolation of effective dosages in mice and other test animals to humans are known. The precise dose will depend upon a number of factors, including whether the antibody is for diagnosis, prevention or for treatment, the size and location of the area to be treated, the precise nature of the antibody (e.g. whole antibody, fragment or diabody), and the nature of any detectable label or other molecule attached to the antibody. A typical antibody dose will be in the range 100 μg to 1 g for systemic applications, and 1 μg to 1 mg for topical applications. An initial higher loading dose, followed by one or more lower doses, may be administered. Typically, the antibody will be a whole antibody, e.g. the IgG1 isotype. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician. Treatments may be every two to four weeks for subcutaneous administration and every four to eight weeks for intravenous administration. In some embodiments of the present invention, treatment is periodic, and the period between administrations is about two weeks or more, e.g. about three weeks or more, about four weeks or more, or about once a month. In other embodiments of the invention, treatment may be given before, and/or after surgery, and may be administered or applied directly at the anatomical site of surgical treatment.

The invention is also directed to a method of producing a stable, aqueous antibody formulation, the method comprising: purifying an antibody to about 100 mg/mL to about 200 mg/mL of an antibody or fragment thereof that specifically binds human interleukin-4 receptor alpha (hIL-4Rα) as described herein, then placing the isolated antibody in a stabilizing formulation to form the stable, aqueous antibody formulation, wherein the resulting stable, aqueous antibody formulation comprises: (1) about 100 mg/mL to about 200 mg/mL of the antibody; (2) about 50 mM to about 400 mM of a viscosity modifier; (3) about 0.01% to about 0.2% of a non-ionic surfactant; and (4) a formulation buffer. In some embodiments, the antibody is concentrated in the presence of trehalose, arginine, or combinations thereof. In some embodiments, the trehalose, arginine, or combinations thereof is added to aid the tangential flow filtration process.

In certain embodiments the invention is directed to the following:
1. A stable antibody formulation comprising:
   a. about 100 mg/mL to about 200 mg/mL of an antibody or fragment thereof that specifically binds human interleukin-4 receptor alpha (hIL-4Rα), wherein:
      (I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
         HCDR1 has amino acid sequence SEQ ID NO: 193;
         HCDR2 has amino acid sequence SEQ ID NO: 194;
         HCDR3 has amino acid sequence SEQ ID NO: 195;
         LCDR1 has amino acid sequence SEQ ID NO: 198;
         LCDR2 has amino acid sequence SEQ ID NO: 199; and
         LCDR3 has amino acid sequence SEQ ID NO: 200;
      (II)
         the HCDR1 has amino acid sequence SEQ ID NO: 363;
         the HCDR2 has amino acid sequence SEQ ID NO: 364;
         the HCDR3 has amino acid sequence SEQ ID NO: 365;
         the LCDR1 has amino acid sequence SEQ ID NO: 368;
         the LCDR2 has amino acid sequence SEQ ID NO: 369; and
         the LCDR3 has amino acid sequence SEQ ID NO: 370;
      OR
         the HCDR1 has amino acid sequence SEQ ID NO: 233;
         the HCDR2 has amino acid sequence SEQ ID NO: 234;
         the HCDR3 has amino acid sequence SEQ ID NO: 235;
         the LCDR1 has amino acid sequence SEQ ID NO: 238;
         the LCDR2 has amino acid sequence SEQ ID NO: 239; and
         the LCDR3 has amino acid sequence SEQ ID NO: 240;
      (III) the antibody comprises a VH domain wherein:
         i. the VH domain has amino acid sequence SEQ ID NO: 192;
         ii. the VH domain has amino acid sequence SEQ ID NO: 362; or
         iii. the VH domain has amino acid sequence SEQ ID NO: 232; and,
      wherein the VH domain comprises one or more amino acid substitutions at the following residues within the framework regions, using the standard numbering of Kabat:
         11, 12 in HFW1;
         37, 48 in HFW2;
         68, 84, 85 in HFW3; or
         105, 108, 113 in HFW4;
      (IV) the antibody comprises a VL domain wherein:
         i. the VL domain has amino acid sequence SEQ ID NO: 197;
         ii. the VL domain has amino acid sequence SEQ ID NO: 367; or
         iii. the VL domain has amino acid sequence SEQ ID NO: 237; and,
      wherein the VL domain comprises one or more amino acid substitutions at the following residues within the framework regions, using the standard numbering of Kabat:
         1, 2, 3, 9 in LFW1;
         38, 42 in LFW2; or
         58, 65, 66, 70, 74, 85, 87 in LFW3;
      OR
      (V) wherein the antibody or fragment thereof comprises a VH and a VL domain wherein:
         i. the VH domain has amino acid sequence SEQ ID NO: 192 and the VL domain has amino acid sequence SEQ ID NO: 197;
         ii. the VH domain has amino acid sequence SEQ ID NO: 362 and the VL domain has amino acid sequence SEQ ID NO: 367; or
         iii. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
         11, 12 in HFW1;
         37, 48 in HFW2;
         68, 84, 85 in HFW3;
         105, 108, 113 in HFW4;
         1, 2, 3, 9 in LFW1;
         38, 42 in LFW2; or
         58, 65, 66, 70, 74, 85, 87 in LFW3;
      or any combination of (I)-(V); and
   b. about 50 mM to about 400 mM of a viscosity modifier;
   c. about 0.002% to about 0.2% of a non-ionic surfactant; and
   d. a formulation buffer.
2. The antibody formulation of claim 1, wherein the formulation buffer is essentially free of phosphate.
3. The antibody formulation of claim 2, wherein the viscosity modifier is selected from the group consisting of histidine, arginine, lysine, polyvinyl alcohol, polyalkyl cellulose, hydroxyalkyl cellulose, glycerin, polyethylene glycol, glucose, dextrose, and sucrose.
4. The antibody formulation of any one of claims 1 to 3, wherein the viscosity modifier is lysine, arginine, or histidine.
5. The antibody formulation of claim 4, wherein the viscosity modifier is arginine.
6. The antibody formulation of any one of claims 1 to 4, wherein the viscosity modifier is in a concentration of about 100 mM to about 250 mM.
7. The antibody formulation of any one of claims 1 to 5, wherein the viscosity modifier is in a concentration of about 190 mM.
8. The antibody formulation of any one of claims 1 to 7, wherein the non-ionic surfactant is selected from the group consisting of Triton X-100, Tween 80, polysorbate 20, polysorbate 80, nonoxynol-9, polyoxamer, stearyl alcohol, or sorbitan monostearate.
9. The antibody formulation of claim 8, wherein the non-ionic surfactant is polysorbate 80.
10. The antibody formulation of any one of claims 1 to 9, wherein formulation comprises about 0.02% to about 0.08% of a non-ionic surfactant.
11. The antibody formulation of claim 10, wherein formulation comprises about 0.04% of a non-ionic surfactant.
12. The antibody formulation of any one of claims 1 to 11, wherein the formulation buffer is an acetate buffer, TRIS buffer, HEPES buffer, hydrochloride buffer, arginine buffer, glycine buffer, citrate buffer, or TES buffer.
13. The antibody formulation of claim 12, wherein the formulation buffer is an arginine buffer.
14. The antibody formulation of claim 13, wherein the arginine buffer comprises arginine hydrochloride.
15. The antibody formulation of claim 14, wherein the arginine buffer further comprises histidine.
16. The antibody formulation of claim 15, wherein the histidine is L-histidine/L-histidine hydrochloride.
17. The antibody formulation of claim 16, wherein the arginine buffer comprises about 10 mM to about 40 mM L-histidine/L-histidine hydrochloride.
18. The antibody formulation of claim 17, wherein the arginine buffer comprises about 25 mM L-histidine/L-histidine hydrochloride.
19. The antibody formulation of any one of claims 1 to 18, wherein the formulation further comprises about 100 mM to about 200 mM NaCl.
20. The antibody formulation of any one of claims 1 to 19, wherein the formulation has a pH of about 5 to about 8.
21. The antibody formulation of claim 20, wherein the formulation has a pH of about 6.
22. The antibody formulation of claim 1, wherein the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
   HCDR1 has amino acid sequence SEQ ID NO: 193;
   HCDR2 has amino acid sequence SEQ ID NO: 194;
   HCDR3 has amino acid sequence SEQ ID NO: 195;
   LCDR1 has amino acid sequence SEQ ID NO: 198;
   LCDR2 has amino acid sequence SEQ ID NO: 199; and
   LCDR3 has amino acid sequence SEQ ID NO: 200.
23. The antibody formulation of claim 22, wherein the amino acid substitutions comprise one or more substitutions as shown in FIGS. 15 and 16.
24. The antibody formulation of claim 22, wherein the amino acid substitutions comprise an amino acid substitution at one or more of the following residues within the CDRs, using the standard numbering of Kabat:
   53, 57, in HCDR2;
   97, 98, 99, 101, 102 in HCDR3;
   27, 27A, 27B, 31 in LCDR1;
   56 in LCDR2; or
   92, 93, 94, 95, 95A 95B, 95C, 96, 97 in LCDR3.
25. The antibody formulation of claim 22, which in addition comprises one or more amino acid substitutions at the following residues within the framework regions, using the standard numbering of Kabat:
   11, 12 in HFW1;
   37, 48 in HFW2;
   68, 84, 85 in HFW3;
   105, 108, 113 in HFW4;
   1, 2, 3, 9 in LFW1;
   38, 42 in LFW2; or
   58, 65, 66, 70, 74, 85, 87 in LFW3.
26. The antibody formulation of claim 25, wherein the amino acid substitutions in the framework regions comprise one or more substitutions as shown in FIGS. 15 and 16.
27. The antibody formulation of claim 1, wherein the antibody or fragment thereof specifically binds human interleukin-4 receptor alpha (hIL-4Rα), wherein:
   (I)
      the HCDR1 has amino acid sequence SEQ ID NO: 363;
      the HCDR2 has amino acid sequence SEQ ID NO: 364;
      the HCDR3 has amino acid sequence SEQ ID NO: 365;
      the LCDR1 has amino acid sequence SEQ ID NO: 368;
      the LCDR2 has amino acid sequence SEQ ID NO: 369; and
      the LCDR3 has amino acid sequence SEQ ID NO: 370;
   OR
   (II)
      the HCDR1 has amino acid sequence SEQ ID NO: 233;
      the HCDR2 has amino acid sequence SEQ ID NO: 234;
      the HCDR3 has amino acid sequence SEQ ID NO: 235;
      the LCDR1 has amino acid sequence SEQ ID NO: 238;
      the LCDR2 has amino acid sequence SEQ ID NO: 239; and
      the LCDR3 has amino acid sequence SEQ ID NO: 240.
28. The antibody formulation of claim 22 or 27, wherein the antibody or fragment thereof comprises an antibody VH domain and an antibody VL domain, wherein the VH domain comprises HCDR1, HCDR2, HCDR3 and a first framework and the VL domain comprises LCDR1, LCDR2, LCDR3 and a second framework.
29. The antibody formulation of any one of claims 1 to 28, wherein the antibody is an scFv.
30. The antibody formulation of any one of claims 1 to 29, wherein the antibody comprises an antibody constant region.
31. The antibody formulation of any one of claims 1 to 30, wherein the antibody molecule is an IgG1, IgG2 or IgG4 molecule.
32. The antibody formulation of claim 1, wherein the antibody or fragment thereof comprises a VH domain wherein:
   a. the VH domain has amino acid sequence SEQ ID NO: 192;
   b. the VH domain has amino acid sequence SEQ ID NO: 362; or
   c. the VH domain has amino acid sequence SEQ ID NO: 232; and,
      wherein the VH domain comprises one or more amino acid substitutions at the following residues within the framework regions, using the standard numbering of Kabat:
   11, 12 in HFW1;
   37, 48 in HFW2;
   68, 84, 85 in HFW3; or
   105, 108, 113 in HFW4.
33. The antibody formulation of claim 32, wherein the amino acid substitutions in the framework regions comprise one or more substitutions as shown in FIGS. 15 and 16.
34. The antibody formulation of claim 32, wherein the antibody is an scFv.
35. The antibody formulation of claim 32, wherein the antibody comprises an antibody constant region.
36. The antibody formulation of claim 32, wherein the antibody molecule is an IgG1, IgG2 or IgG4 molecule.
37. The antibody formulation of claim 1, wherein the antibody or fragment thereof comprises a VL domain wherein:
   a. the VL domain has amino acid sequence SEQ ID NO: 197;
   b. the VL domain has amino acid sequence SEQ ID NO: 367; or
   c. the VL domain has amino acid sequence SEQ ID NO: 237; and,
      wherein the VL domain comprises one or more amino acid substitutions at the following residues within the framework regions, using the standard numbering of Kabat:
   1, 2, 3, 9 in LFW1;
   38, 42 in LFW2; or
   58, 65, 66, 70, 74, 85, 87 in LFW3.
38. The antibody formulation of claim 37, wherein the antibody molecule is an scFv.
39. The antibody formulation of claim 37, wherein the antibody molecule comprises an antibody constant region.
40. The antibody formulation of claim 37, wherein the antibody molecule is an IgG1, IgG2 or IgG4 molecule.
41. The antibody formulation of claim 1, wherein the antibody or fragment thereof comprises a VH and a VL domain wherein:
   a. the VH domain has amino acid sequence SEQ ID NO: 192 and the VL domain has amino acid sequence SEQ ID NO: 197;
   b. the VH domain has amino acid sequence SEQ ID NO: 362 and the VL domain has amino acid sequence SEQ ID NO: 367; or
   c. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
   11, 12 in HFW1;
   37, 48 in HFW2;
   68, 84, 85 in HFW3;
   105, 108, 113 in HFW4;
   1, 2, 3, 9 in LFW1;
   38, 42 in LFW2; or
   58, 65, 66, 70, 74, 85, 87 in LFW3.
42. The antibody formulation of claim 41, wherein the amino acid substitutions in the framework regions comprise one or more substitutions as shown in FIGS. 15 and 16.
43. The antibody formulation of claim 41, wherein the antibody molecule is an scFv.
44. The antibody formulation of claim 41, wherein the antibody molecule comprises an antibody constant region.
45. The antibody formulation of claim 41, wherein the antibody molecule is an IgG1, IgG2 or IgG4 molecule.
46. The antibody formulation of any one of claims 1 to 45, wherein said antibody was not subjected to lyophilization.
47. The antibody formulation of any one of claims 1 to 46, wherein said formulation is stable upon storage at about 40° C. for at least 1 month.
48. The antibody formulation of any one of claims 1 to 47, wherein the formulation has less than 1000 “≧10 μm particles”/mL after storage at about 40° C. for 1 month.
49. The antibody formulation of any one of claims 1 to 48, wherein the formulation has a viscosity of less than 20 cP at 23° C.
50. A stable antibody formulation comprising:
   a. about 100 mg/mL to about 200 mg/mL of an antibody or fragment thereof that specifically binds human interleukin-4 receptor alpha (hIL-4Rα), wherein:
      (I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
         HCDR1 has amino acid sequence SEQ ID NO: 193;
         HCDR2 has amino acid sequence SEQ ID NO: 194;
         HCDR3 has amino acid sequence SEQ ID NO: 195;
         LCDR1 has amino acid sequence SEQ ID NO: 198;
         LCDR2 has amino acid sequence SEQ ID NO: 199; and
         LCDR3 has amino acid sequence SEQ ID NO: 200;
      (II)
         the HCDR1 has amino acid sequence SEQ ID NO: 363;
         the HCDR2 has amino acid sequence SEQ ID NO: 364;
         the HCDR3 has amino acid sequence SEQ ID NO: 365;
         the LCDR1 has amino acid sequence SEQ ID NO: 368;
         the LCDR2 has amino acid sequence SEQ ID NO: 369; and
         the LCDR3 has amino acid sequence SEQ ID NO: 370;
      OR
         the HCDR1 has amino acid sequence SEQ ID NO: 233;
         the HCDR2 has amino acid sequence SEQ ID NO: 234;
         the HCDR3 has amino acid sequence SEQ ID NO: 235;
         the LCDR1 has amino acid sequence SEQ ID NO: 238;
         the LCDR2 has amino acid sequence SEQ ID NO: 239; and
         the LCDR3 has amino acid sequence SEQ ID NO: 240;
      (III) the antibody comprises a VH domain wherein:
         i. the VH domain has amino acid sequence SEQ ID NO: 192;
         ii. the VH domain has amino acid sequence SEQ ID NO: 362; or
         iii. the VH domain has amino acid sequence SEQ ID NO: 232; and,
      wherein the VH domain comprises one or more amino acid substitutions at the following residues within the framework regions, using the standard numbering of Kabat:
         11, 12 in HFW1;
         37, 48 in HFW2;
         68, 84, 85 in HFW3; or
         105, 108, 113 in HFW4;
      (IV) the antibody comprises a VL domain wherein:
         iv. the VL domain has amino acid sequence SEQ ID NO: 197;
         v. the VL domain has amino acid sequence SEQ ID NO: 367; or
         vi. the VL domain has amino acid sequence SEQ ID NO: 237; and,
      wherein the VL domain comprises one or more amino acid substitutions at the following residues within the framework regions, using the standard numbering of Kabat:
         1, 2, 3, 9 in LFW1;
         38, 42 in LFW2; or
         58, 65, 66, 70, 74, 85, 87 in LFW3;
      OR
      (V) wherein the antibody or fragment thereof comprises a VH and a VL domain wherein:
         iv. the VH domain has amino acid sequence SEQ ID NO: 192 and the VL domain has amino acid sequence SEQ ID NO: 197;
         v. the VH domain has amino acid sequence SEQ ID NO: 362 and the VL domain has amino acid sequence SEQ ID NO: 367; or
         vi. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
         11, 12 in HFW1;
         37, 48 in HFW2;
         68, 84, 85 in HFW3;
         105, 108, 113 in HFW4;
         1, 2, 3, 9 in LFW1;
         38, 42 in LFW2; or
         58, 65, 66, 70, 74, 85, 87 in LFW3;
      or any combination of (I)-(V); and
   b. about 50 mM to about 400 mM arginine;
   c. about 0.002% to about 0.2% polysorbate 80; and
   d. about 10 to about 40 mM L-histidine/L-histidine hydrochloride.
51. A stable antibody formulation comprising:
   a. about 100 mg/mL to about 200 mg/mL of an antibody or fragment thereof that specifically binds human interleukin-4 receptor alpha (hIL-4Rα), wherein:
      (I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
         HCDR1 has amino acid sequence SEQ ID NO: 193;
         HCDR2 has amino acid sequence SEQ ID NO: 194;
         HCDR3 has amino acid sequence SEQ ID NO: 195;
         LCDR1 has amino acid sequence SEQ ID NO: 198;
         LCDR2 has amino acid sequence SEQ ID NO: 199; and
         LCDR3 has amino acid sequence SEQ ID NO: 200;
      (II)
         the HCDR1 has amino acid sequence SEQ ID NO: 363;
         the HCDR2 has amino acid sequence SEQ ID NO: 364;
         the HCDR3 has amino acid sequence SEQ ID NO: 365;
         the LCDR1 has amino acid sequence SEQ ID NO: 368;
         the LCDR2 has amino acid sequence SEQ ID NO: 369; and
         the LCDR3 has amino acid sequence SEQ ID NO: 370;
      OR
         the HCDR1 has amino acid sequence SEQ ID NO: 233;
         the HCDR2 has amino acid sequence SEQ ID NO: 234;
         the HCDR3 has amino acid sequence SEQ ID NO: 235;
         the LCDR1 has amino acid sequence SEQ ID NO: 238;
         the LCDR2 has amino acid sequence SEQ ID NO: 239; and
         the LCDR3 has amino acid sequence SEQ ID NO: 240;
      (III) the antibody comprises a VH domain wherein:
         iv. the VH domain has amino acid sequence SEQ ID NO: 192;
         v. the VH domain has amino acid sequence SEQ ID NO: 362; or
         vi. the VH domain has amino acid sequence SEQ ID NO: 232; and,
      wherein the VH domain comprises one or more amino acid substitutions at the following residues within the framework regions, using the standard numbering of Kabat:
         11, 12 in HFW1;
         37, 48 in HFW2;
         68, 84, 85 in HFW3; or
         105, 108, 113 in HFW4;
      (IV) the antibody comprises a VL domain wherein:
         iv. the VL domain has amino acid sequence SEQ ID NO: 197;
         v. the VL domain has amino acid sequence SEQ ID NO: 367; or
         vi. the VL domain has amino acid sequence SEQ ID NO: 237; and,
      wherein the VL domain comprises one or more amino acid substitutions at the following residues within the framework regions, using the standard numbering of Kabat:
         1, 2, 3, 9 in LFW1;
         38, 42 in LFW2; or
         58, 65, 66, 70, 74, 85, 87 in LFW3;
      OR
      (V) wherein the antibody or fragment thereof comprises a VH and a VL domain wherein:
         iv. the VH domain has amino acid sequence SEQ ID NO: 192 and the VL domain has amino acid sequence SEQ ID NO: 197;
         v. the VH domain has amino acid sequence SEQ ID NO: 362 and the VL domain has amino acid sequence SEQ ID NO: 367; or
         vi. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
         11, 12 in HFW1;
         37, 48 in HFW2;
         68, 84, 85 in HFW3;
         105, 108, 113 in HFW4;
         1, 2, 3, 9 in LFW1;
         38, 42 in LFW2; or
         58, 65, 66, 70, 74, 85, 87 in LFW3;
      or any combination of (I)-(V); and
   b. about 190 mM arginine;
   c. about 0.04% polysorbate 80; and
   d. about 25 mM L-histidine/L-histidine hydrochloride.
52. The antibody formulation of any one of claims 1 to 51, wherein the formulation is stable upon storage at about 25° C. for at least 3 months.
53. The antibody formulation of any one of claims 1 to 52, wherein the formulation is stable upon storage at about 5° C. for at least 18 months.
54. The antibody formulation of any one of claims 1 to 53, wherein the antibody stored at about 40° C. for at least 1 month retains at least 80% of binding ability to an hIL-4Rα polypeptide compared to a reference antibody which has not been stored.
55. The antibody formulation of any one of claims 1 to 54, wherein the antibody stored at about 5° C. for at least 6 months retains at least 80% of binding ability to an hIL-4Rα polypeptide compared to a reference antibody which has not been stored.
56. The antibody formulation of any one of claims 1 to 55, wherein the antibody stored at about 40° C. for at least 1 month retains at least 50% of binding ability to an hIL-4Rα polypeptide compared to a reference antibody which has not been stored.
57. The antibody formulation of any one of claims 1 to 56, wherein the antibody stored at about 5° C. for at least 6 months retains at least 50% of binding ability to an hIL-4Rα polypeptide compared to a reference antibody which has not been stored.
58. The antibody formulation of any one of claims 1 to 57, wherein the formulation is an injectable formulation.
59. The antibody formulation of any one of claims 1 to 58, wherein the formulation is suitable for intravenous, subcutaneous, or intramuscular administration.
60. A sealed container containing the antibody formulation of any one of claims 1 to 59.
61. A pharmaceutical unit dosage form suitable for parenteral administration to a human which comprises the antibody formulation of any one of claims 1 to 59 in a suitable container.
62. The pharmaceutical unit dosage form of claim 61, wherein the antibody formulation is administered intravenously, subcutaneously, or intramuscularly.
63. The pharmaceutical unit dosage form of claim 61 or 62, wherein the suitable container is a pre-filled syringe.
64. A kit comprising the formulation of any one of claims 1 to 59, the container of claim 60, the unit dosage form of any one of claims 61 to 62, or the pre-filled syringe of claim 63.
65. A method of producing a stable, aqueous antibody formulation, the method comprising:
   a. purifying an antibody to about 100 mg/mL to about 200 mg/mL of an antibody or fragment thereof that specifically binds human interleukin-4 receptor alpha (hIL-4Rα), wherein:
      (I) the antibody comprises a set of CDRs: HCDR1, HCDR2, HCDR3, LCDR1, LCDR2 and LCDR3, wherein the set of CDRs has 10 or fewer amino acid substitutions from a reference set of CDRs in which:
         HCDR1 has amino acid sequence SEQ ID NO: 193;
         HCDR2 has amino acid sequence SEQ ID NO: 194;
         HCDR3 has amino acid sequence SEQ ID NO: 195;
         LCDR1 has amino acid sequence SEQ ID NO: 198;
         LCDR2 has amino acid sequence SEQ ID NO: 199; and
         LCDR3 has amino acid sequence SEQ ID NO: 200;
      (II)
         the HCDR1 has amino acid sequence SEQ ID NO: 363;
         the HCDR2 has amino acid sequence SEQ ID NO: 364;
         the HCDR3 has amino acid sequence SEQ ID NO: 365;
         the LCDR1 has amino acid sequence SEQ ID NO: 368;
         the LCDR2 has amino acid sequence SEQ ID NO: 369; and
         the LCDR3 has amino acid sequence SEQ ID NO: 370;
      OR
         the HCDR1 has amino acid sequence SEQ ID NO: 233;
         the HCDR2 has amino acid sequence SEQ ID NO: 234;
         the HCDR3 has amino acid sequence SEQ ID NO: 235;
         the LCDR1 has amino acid sequence SEQ ID NO: 238;
         the LCDR2 has amino acid sequence SEQ ID NO: 239; and
         the LCDR3 has amino acid sequence SEQ ID NO: 240;
      (III) the antibody comprises a VH domain wherein:
         the VH domain has amino acid sequence SEQ ID NO: 192;
         the VH domain has amino acid sequence SEQ ID NO: 362; or
         the VH domain has amino acid sequence SEQ ID NO: 232; and,
      wherein the VH domain comprises one or more amino acid substitutions at the following residues within the framework regions, using the standard numbering of Kabat:
         11, 12 in HFW1;
         37, 48 in HFW2;
         68, 84, 85 in HFW3; or
         105, 108, 113 in HFW4;
      (IV) the antibody comprises a VL domain wherein:
         the VL domain has amino acid sequence SEQ ID NO: 197;
         the VL domain has amino acid sequence SEQ ID NO: 367; or
         the VL domain has amino acid sequence SEQ ID NO: 237; and,
      wherein the VL domain comprises one or more amino acid substitutions at the following residues within the framework regions, using the standard numbering of Kabat:
         1, 2, 3, 9 in LFW1;
         38, 42 in LFW2; or
         58, 65, 66, 70, 74, 85, 87 in LFW3;
      OR
      (V) wherein the antibody or fragment thereof comprises a VH and a VL domain wherein:
         i. the VH domain has amino acid sequence SEQ ID NO: 192 and the VL domain has amino acid sequence SEQ ID NO: 197;
         ii. the VH domain has amino acid sequence SEQ ID NO: 362 and the VL domain has amino acid sequence SEQ ID NO: 367; or
         iii. the VH domain has amino acid sequence SEQ ID NO: 232 and the VL domain has amino acid sequence SEQ ID NO: 237; and,
      wherein the VH domain and VL domain comprise one or more amino acid substitutions at the following residues within the framework regions, using the standard numbering of Kabat:
         11, 12 in HFW1;
         37, 48 in HFW2;
         68, 84, 85 in HFW3;
         105, 108, 113 in HFW4;
         1, 2, 3, 9 in LFW1;
         38, 42 in LFW2; or
         58, 65, 66, 70, 74, 85, 87 in LFW3;
      or any combination of (I)-(V); and
   b. placing the isolated antibody in a stabilizing formulation to form the stable, aqueous antibody formulation, wherein the resulting stable, aqueous antibody formulation comprises:
      i. about 100 mg/mL to about 200 mg/mL of the antibody;
      ii. about 50 mM to about 400 mM of a viscosity modifier;
      iii. about 0.002% to about 0.2% of a non-ionic surfactant; and
      iv. a formulation buffer.
66. The method of claim 65, wherein the antibody is concentrated in the presence of trehalose, arginine, or combinations thereof.
67. A method of treating a pulmonary disease or disorder or a chronic inflammatory skin disease or disorder in a subject, the method comprising administering a therapeutically effective amount of the antibody formulation of any one of claims 1 to 60.
68. The method of claim 67, wherein the disease or disorder is selected from the group consisting of asthma, COPD (including chronic bronchitis, small airway disease and emphysema), inflammatory bowel disease, fibrotic conditions (including systemic sclerosis, pulmonary fibrosis, parasite-induced liver fibrosis, and cystic fibrosis, allergy (including for example atopic dermatitis and food allergy), transplantation therapy to prevent transplant rejection, as well as suppression of delayed-type hypersensitivity or contact hypersensitivity reactions, as adjuvants to allergy immunotherapy and as vaccine adjuvants.
69. The method of claim 67, wherein the pulmonary disease or disorder is asthma, COPD, eosinophilic asthma, combined eosinophilic and neutrophilic asthma, aspirin sensitive asthma, allergic bronchopulmonary aspergillosis, acute and chronic eosinophilic bronchitis, acute and chronic eosinophilic pneumonia, Churg-Strauss syndrome, hypereosinophilic syndrome, drug, irritant and radiation-induced pulmonary eosinophilia, infection-induced pulmonary eosinophilia (fungi, tuberculosis, parasites), autoimmune-related pulmonary eosinophilia, eosinophilic esophagitis, Crohn's disease, or combination thereof.
70. The method of claim 69, wherein the pulmonary disease or disorder is asthma.
71. The method of claim 67, wherein the chronic inflammatory skin disorder is selected from the group consisting of atopic dermatitis, allergic contact dermatitis, eczema or psoriasis.
72. The method of claim 71, wherein the inflammatory skin disorder is atopic dermatitis.

### EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

### EXAMPLES

The invention is now described with reference to the following examples. These examples are illustrative only and the invention should in no way be construed as being limited to these examples but rather should be construed to encompass any and all variations which become evident as a result of the teachings provided herein.

### Example 1

### Materials and Methods

### Materials

All the materials used were of USP or Multicompendial grade. All the solutions and buffers were prepared using USP or HPLC water and were filtered through 0.2 μm PVDF filters (Millipore) before further use. Purified anti-hIL-4Rα antibody was purified as summarized in Table 1. Purified anti-hIL-4Rα antibody samples for stability studies were prepared under sterile aseptic conditions in the Biosafety Cabinet Hood (BSC). Bulk material was stored at 2-8° C.

TABLE 1

| Batch number | Scale | Purification process |
|---|---|---|
| SP09-339 | 250 L | MabSelect SuRe (w/ wash); Low pH, |
| | | Fractogel, TFF |
| SP10-383 | 22 L | MabSelect SuRe (w/ wash); Low pH, Poros |
| | | HS50, Chromasorb, Virus filtration, TFF |
| SP13-108 | 50 L | MabSelect SuRe (w/ wash); Low pH, Poros |
| | | HS50, Chromasorb, Virus filtration, TFF |
| SP13-406 | 5 L | MabSelect SuRe (w/ wash); Low pH, Poros |
| | | HS50, Mustang Q, Virus filtration, TFF |
| SP13-118 | 100 L | MabSelect SuRe (w/ wash); Low pH, Poros |
| | | HS50, Mustang Q, Virus filtration, TFF |

### Protein Concentration Determination

Anti-hIL-4Rα protein concentrations were determined by measuring absorbance at 280 nm with an Agilent UV-Vis spectrophotometer as per current formulation sciences guidelines. Dilutions were made with PBS or formulation buffer. An extinction coefficient of 1.77 (mg/mL)⁻¹ cm⁻¹ was used to calculate protein concentrations for all studies. This figure corresponds to the theoretical extinction coefficients determined for the molecule. Where material was constrained, the absorbance at 280 nm was measured using the Nanodrop 2000 (ThermoScientific).

### Purity Determination by Size Exclusion Chromatography (HPSEC)

SEC analysis was performed on an Agilent HPLC system with a TSK-Gel G3000 as per the current Formulation Sciences guidelines. Injection volumes were adjusted to maintain a constant mass of 250 μg for concentrations below 10 mg/mL but greater than 2.5 mg/mL. The diluent used for HPSEC was Phosphate buffered saline (Sigma) or formulation buffer.

### Visual Appearance

Visual inspection of the samples was performed by examining the samples in their respective container for particles using particle standards following procedures adapted from the PhEur (sections 2.9.20).

### Sub-Visible Particle Analysis

Sub-visible particles analysis was performed using either light obscuration Flow microscopy (Brightwell Microflow Imager, MFI) using the current Formulation Sciences guidelines.

### Osmolality

Osmolality was measured on a Gonotec Osmomat 030-D Osmometer freezing point depression osmometer. System suitability was assessed by running a reference standard.

### Viscosity Assessment

The viscosities of anti-hIL-4Rα formulations at various concentrations were measured using an Anton Paar MCR301 Rheometer with cone and plate accessory (40 mm). Viscosities were reported at the high-shear limit of 1000 per second shear rate.

### Formulation Stability Studies

Anti-hIL-4Rα antibody formulated with different excipients was filled into clear 3 cc, 13 mm glass vials. For accelerated screening, samples were placed on stability at 40° C./75% RH. For longer-term stability studies of lead formulations, in addition to the accelerated 40° C. condition, studies were also performed at 25° C./60% RH and 5° C. Samples were analyzed by SEC HPLC and Bioanalyzer and the vials were visually inspected for particles. In addition selected timepoints were analyzed for potency, osmolality, pH, and microflow imaging (MFI) as appropriate.

### Thermal Stability Using Differential Scanning Calorimetry

Differential scanning calorimetry (DSC) experiments were performed on a VP-DSC Ultrasensitive Differential scanning calorimeter (Microcal, Northampton, Mass.) using 96 well plate at a protein concentration of 5 mg/mL. Samples were heated from 25-100° C. at a rate of 95° C. per hour. Normalized heat capacity (Cp) data were corrected for buffer baseline.

### Example 2

### Stability 50 mg/ml Screening Assessments

The stability of multiple anti-hIL-4Rα antibody formulations were assessed and found to be to be comparable from a stability perspective. Conformational (thermal) stability and aggregation rate at a stress temperature of 40° C. were the main parameters investigated in this study.

Table 2 summarizes an investigation into the impact of buffer type, sugar type, sugar level and arginine-HCL level on the conformational stability (Tm1) and aggregation rate/month at 40° C. of anti-hIL-4Rα antibody formulations at a concentration of approximately 50 mg/mL.

TABLE 2

| | | | | Aggregation |
|---|---|---|---|---|
| | | Osmo. | Measured | Rate at |
| Ab conc. | | (mosm/ | Tm1 | 40° C./ |
| (mg/mL) | Formulation | kg) | (° C.) | (%/month) |
| 66.1 | 1) 20 mM Citrate, | 400 | 61.8 | 1.7 |
| | 290 mM Sucrose, | | | |
| | pH 6.0 | | | |
| 53.3 | 2) 20 mM Histidine, | 320 | 59.4 | 2.0 |
| | 260 mM Trehalose, | | | |
| | pH 6.0 | | | |
| 48.9 | 3) 20 mM Histidine, | 431 | 58.1 | 1.6 |
| | 260 mM Trehalose, | | | |
| | 50 mM Arginine, pH6.0 | | | |
| 54.9 | 4) 20 mM Histidine, | 480 | 58.2 | 1.7 |
| | 290 mM Sucrose, 50 mM | | | |
| | Arginine, pH6.0 | | | |
| 57.6 | 5) 20 mM Histidine, | 404 | 57.9 | 1.5 |
| | 175 mM Sucrose, | | | |
| | 100 mM Arginine, | | | |
| | pH6.0 | | | |

Table 2 shows that all anti-hIL-4Rα antibody formulations have comparable conformational stability (Tm1) and aggregation rates after 1 month incubation at 40° C. Samples 2 and 3 show that the addition of Arginine-HCL does not impact the conformational stability or the aggregation rate. Samples 4 and 5 show that increasing the concentration of Arginine-HCL does not improve the conformational stability or the aggregation rate at 40° C.

### Example 3

### Viscosity Screening Assessment

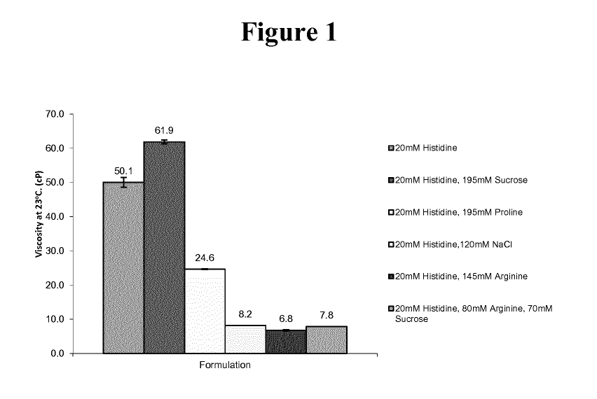

The viscosity of anti-hIL-4Rα antibody at concentrations of 109.8 mg/ml±5.8 mg/ml in multiple formulations was assessed. FIG. 1 shows that the viscosity of anti-hIL-4Rα antibody in a histidine base buffer formulation and a sucrose containing formulation is >50 cP at 23° C. The data shows that an ionic excipient is necessary to reduce the viscosity of anti-hIL-4Rα antibody at high concentration to a level that would be appropriate for subcutaneous delivery. Previous data suggests that this level would be <20 cP at 23° C.

### Example 4

### Stability High Concentration Screening Assessment

The Stability and viscosity of anti-hIL-4Rα antibody was assessed in Histidine/Arginine-HCL formulations over a narrow pH range. This experiment was designed to show robustness in stability and viscosity over the pH range that is covered in the product specifications to allow for manufacturing limits. Table 3 shows that stability and viscosity are within acceptable limits and robust over the range of pH 6.0±0.5.

Purity loss is based on 10 months data at 2-8° C. and extrapolated to calculate a yearly loss of purity as measured by HPSEC (High performance size exclusion chromatography).

TABLE 3

| | | | | Purity | Viscosity |
|---|---|---|---|---|---|
| | | Antibody | Measured | loss at | at |
| | | conc. | Tm1 | 2-8 C./ | 23° C. |
| pH | Formulation | (mg/ml) | (° C.) | year | (cP) |
| 1) 5.5 | 25 mM Histidine, | 142.8 | 52.0 | 0.7 | 10 |
| | 190 mM Arginine, | | | | |
| | 0.02% PS80 | | | | |
| 2) 6.0 | 25 mM Histidine, | 153.9 | 56.1 | 0.7 | 11 |
| | 190 mM Arginine, | | | | |
| | 0.02% PS80 | | | | |
| 3) 6.5 | 25 mM Histidine, | 150.2 | 58.4 | 1 | 11 |
| | 190 mM Arginine, | | | | |
| | 0.02% PS80 | | | | |

### Example 5

### IL4R Antibody Sensitivity to Agitation

### Materials

Anti-hIL-4Rα antibody was formulated at a concentration of 140 mg/ml in 25 mM Histidine/Histidine-HCL, 190 mM Arginine-HCL, pH 6. The polysorbate 80 (plant-derived) used was the multicompendial J.T. Baker brand. Water was obtained from an in-house USP water system. All other reagents used were of pharmacopeial grade.

### Sample Preparation

The anti-hIL-4Rα antibody sample was filtered through a 0.22 uM PVDF syringe filter and divided into polypropylene tubes. Polysorbate 80 was added to each sample at varying concentrations as shown in Table 4. Control samples that did not contain polysorbate 80 were also prepared (samples 1-3, Table 4). Each sample was re-filtered through a 0.22 μM syringe filter and aseptically filled into 3 cc glass vials, stoppered and sealed with an aluminum overseal. Vials were either subjected to agitation at 600 rpm for four hours using an orbital shaker (Scientific Industries, Inc) or left upright on the bench for the duration of the experiment. At the end of the agitation time, samples were analyzed by high performance size exclusion chromatography for soluble aggregate content, flow imaging for subvisible particle characterization and visually inspected for the presence of large particles or fibers.

TABLE 4

| | Concentration | Final PS80 concentration and |
|---|---|---|
| Sample number | hIL4R antibody | stress condition. |
| 1 | 0 | 0% PS80, no agitation |
| 2 | 140 | 0% PS80, no agitation |
| 3 | 140 | 0% PS80, agitation |
| 4 | 140 | 0.005% PS80, agitation |
| 5 | 140 | 0.01% PS80. agitation |
| 6 | 140 | 0.02% PS80, agitation |
| 7 | 140 | 0.03% PS80, agitation |
| 8 | 140 | 0.04% PS80, agitation |
| 9 | 140 | 0.05% PS80, agitation |
| 10 | 140 | 0.07% PS80, agitation |

### Purity and Soluble Aggregation.

High Performance Size Exclusion Chromatography (HPSEC) was performed using a TSK-GEL G3000SWXL column and SW guard column (Tosoh Bioscience)) with UV detection at 280 nm. A flow rate of 1.0 mL/min for 20 min using a pH 6.8 mobile phase containing 0.1 M sodium phosphate, 0.1 M sodium sulfate, and 0.05% (w/v) sodium azide was used to assay the samples. About 250 μg of protein was injected. Elution of soluble aggregates, monomer, and fragments occurred at approximately 6 to 8 min, 8.6 min, and 9 to 10 min respectively.

### Visual Inspection.

Particle levels in samples were compared against a series of in-house barium sulfate visible particle standards. The samples in 3 cc glass vials were inspected for the presence of particulate and fibrous matter using a light box with both dark and light background. Samples were assigned as being free from visible particles, practically free from visible particles or many particles.

### Subvisible Particle Analysis

Sub-visible particles analysis was performed using flow microscopy (Brightwell Microflow Imager, MFI) using the current Formulation Sciences guidelines. Samples were analyzed neat and the flow cell was cleaned thoroughly with ultrapure water between each sample.

### Results and Discussion

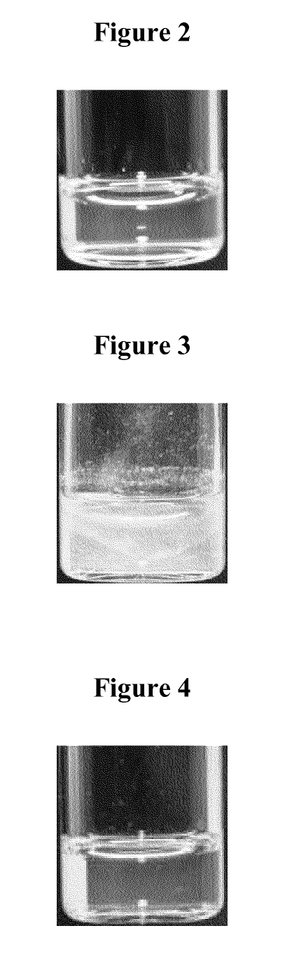

Anti-hIL-4Rα antibody was found to be very sensitive to agitation induced aggregation in the absence of polysorbate 80. Table 5 is a summary of the data from this experiment. Samples 2 and 3 in Table 5 show that agitation in the absence of polysorbate 80 increases the percent soluble aggregate by 2.5 fold and causes a large increase in the number of visible particles and fibers; FIG. 2 and FIG. 3. Sample 3 could not be analyzed by Microflow imaging due to the presence of a high level of precipitate within the sample. These particles could block the flow cell which has a maximum diameter of 100 μm. Samples 4-10 in Table 5 show that the presence of >0.005% PS80 protected the antibody from forming large visible particles upon agitation; FIG. 4. At a level of >0.02% PS80 (samples 6-10), agitated samples have a comparable level of subvisible particles, soluble aggregate and visual appearance to a non-agitated sample (sample 2). These data show that a minimum level of >0.02% PS80 is required in the anti-hIL-4Rα formulation to completely protect the antibody from agitation induced aggregation.

TABLE 5

| | Con- | | | | |
|---|---|---|---|---|---|
| | centra- | Final PS80 | | | |
| | tion | concentra- | | | |
| | hIL4R | tion | Soluble | Number of | |
| Sample | antibody | and stress | aggregate | ≧10 μm | Visual |
| number | (mg/ml) | condition. | (%) | particles | appearance |
| 1 | 0 | 0% PS80, | N/A | 0 | Practically |
| | | no agitation | | | free from |
| | | | | | visible |
| | | | | | particles |
| 2 | 140 | 0% PS80, no | 1.0 | 14 | Practically |
| | | agitation | | | free from |
| | | | | | visible |
| | | | | | particles |
| 3 | 140 | 0% PS80, | 2.5 | N/D | Many |
| | | agitation | | | particles |
| 4 | 140 | 0.005% | 1.4 | 555 | Practically |
| | | PS80, | | | free from |
| | | agitation | | | visible |
| | | | | | particles |
| 5 | 140 | 0.01% PS80. | 1.1 | 83 | Practically |
| | | agitation | | | free from |
| | | | | | visible |
| | | | | | particles |
| 6 | 140 | 0.02% PS80, | 1.0 | 5 | Practically |
| | | agitation | | | free from |
| | | | | | visible |
| | | | | | particles |
| 7 | 140 | 0.03% PS80, | 1.0 | 18 | Practically |
| | | agitation | | | free from |
| | | | | | visible |
| | | | | | particles |
| 8 | 140 | 0.04% PS80, | 1.0 | 9 | Practically |
| | | agitation | | | free from |
| | | | | | visible |
| | | | | | particles |
| 9 | 140 | 0.05% PS80, | 1.0 | 0 | Practically |
| | | agitation | | | free from |
| | | | | | visible |
| | | | | | particles |
| 10 | 140 | 0.07% PS80, | 1.1 | 28 | Practically |
| | | agitation | | | free from |
| | | | | | visible |
| | | | | | particles |

### Example 6

### hIL4R Antibody Sensitivity to Freeze Thaw

### Materials

Anti-hIL-4Rα antibody was formulated at a concentration of 140 mg/ml in 25 mM Histidine/Histidine-HCL, 190 mM Arginine-HCL, pH 6. The polysorbate 80 (plant-derived) used was the multicompendial J.T. Baker brand. Water was obtained from an in-house USP water system. All other reagents used were of pharmacopeial grade.

### Sample Preparation

The anti-hIL-4Rα antibody sample was filtered through a 0.22 uM PVDF syringe filter and divided into polypropylene tubes. Polysorbate 80 was added to each sample at varying concentrations as shown in Table 6. Control samples that did not contain polysorbate 80 were also prepared (samples 1-3, Table 6). Each sample was re-filtered through a 0.22 μM syringe filter and aseptically filled into 3 cc glass vials, stoppered and sealed with an aluminum overseal. Vials were either subjected to 5× uncontrolled freeze thaw (FT) cycling (one cycle consists of 1 hour at −40° C. followed by 1 hour at room temperature) or left upright on the bench for the duration of the experiment. At the end of the freeze thaw cycling, samples were analysed by high performance size exclusion chromatography for soluble aggregate content, flow imaging for subvisible particle characterisation and visually inspected for the presence of large particles or fibers.

TABLE 6

| | Concentration | |
|---|---|---|
| | hIL4R antibody | Final PS80 concentration and |
| Sample number | (mg/ml) | stress condition. |
| 1 | 0 | 0% PS80, no FT |
| 2 | 140 | 0% PS80, no FT |
| 3 | 140 | 0% PS80, 5 X FT |
| 4 | 140 | 0.005% PS80, 5 X FT |
| 5 | 140 | 0.01% PS80. 5 X FT |
| 6 | 140 | 0.02% PS80, 5 X FT |
| 7 | 140 | 0.03% PS80, 5 X FT |
| 8 | 140 | 0.04% PS80, 5 X FT |
| 9 | 140 | 0.05% PS80, 5 X FT |
| 10 | 140 | 0.07% PS80, 5 X FT |

### Purity and Soluble Aggregation.

High Performance Size Exclusion Chromatography (HPSEC) was performed using a TSK-GEL G3000SWXL column and SW guard column (Tosoh Bioscience) with UV detection at 280 nm. A flow rate of 1.0 mL/min for 20 min using a pH 6.8 mobile phase containing 0.1 M sodium phosphate, 0.1 M sodium sulfate, and 0.05% (w/v) sodium azide was used to assay the samples. About 250 μg of protein was injected. Elution of soluble aggregates, monomer, and fragments occurred at approximately 6 to 8 min, 8.6 min, and 9 to 10 min respectively.

### Visual Inspection.

Particle levels in samples were compared against a series of in-house barium sulfate visible particle standards. The samples in 3 cc glass vials were inspected for the presence of particulate and fibrous matter using a light box with both dark and light background. Samples were assigned as being free from visible particles, practically free from visible particles or many particles.

### Subvisible Particle Analysis

Sub-visible particles analysis was performed using flow microscopy (Brightwell Microflow Imager, MFI) using the current Formulation Sciences guidelines. Samples were analyzed neat and the flow cell was cleaned thoroughly with ultrapure water between each sample.

### Results and Discussion

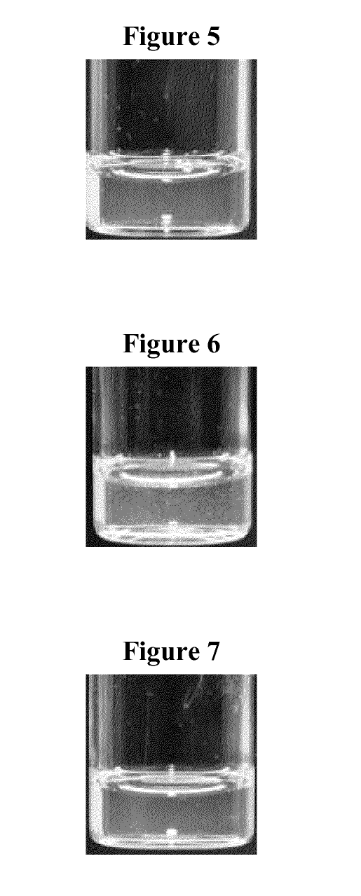

Anti-hIL-4Rα antibody was found to be very sensitive to freeze thaw induced aggregation in the absence of polysorbate 80. Table 7 is a summary of the data from this experiment. Samples 2 and 3 in Table 7 show that freeze thaw cycling in the absence of polysorbate 80 causes a large increase in the number of visible particles and fibers; FIG. 5 and FIG. 6. However the subvisible particle count decreased with freeze thaw cycling in the absence of polysorbate 80 (Samples 2 and 3). This could be due to the presence a high level of large visible particles in sample 3. This could block the flow cell which has a maximum diameter of 100 μm and lead to a lower number of particles available for imaging. Samples 4-10 in Table 7 show that the presence of >0.005% PS80 protected the antibody from forming large visible particles with freeze thaw cycling; FIG. 7. No impact of freeze thaw cycling on soluble aggregate formation (HPSEC) was seen in this experiment.

TABLE 7

| | Con- | | | | |
|---|---|---|---|---|---|
| | centra- | | | | |
| | tion | Final PS80 | | Number | |
| | hIL4R | concentration | Soluble | of | |
| Sample | antibody | (%) and stress | aggregate | ≧10 μm | Visual |
| number | (mg/ml) | condition. | (%) | particles | appearance |
| 1 | 0 | 0% PS80, | N/A | 0 | Practically |
| | | no FT | | | free from |
| | | | | | visible |
| | | | | | particles |
| 2 | 140 | 0% PS80, no | 1.0 | 2891 | Practically |
| | | FT | | | free from |
| | | | | | visible |
| | | | | | particles |
| 3 | 140 | 0% PS80, 5 X | 1.0 | 96 | Many |
| | | FT | | | particles |
| 4 | 140 | 0.005% PS80, | 1.0 | 23 | Practically |
| | | 5 X FT | | | free from |
| | | | | | visible |
| | | | | | particles |
| 5 | 140 | 0.01% PS80. 5 | 1.0 | 0 | Practically |
| | | X FT | | | free from |
| | | | | | visible |
| | | | | | particles |
| 6 | 140 | 0.02% PS80, 5 | 1.0 | 9 | Practically |
| | | X FT | | | free from |
| | | | | | visible |
| | | | | | particles |
| 7 | 140 | 0.03% PS80, 5 | 1.0 | 5 | Practically |
| | | X FT | | | free from |
| | | | | | visible |
| | | | | | particles |
| 8 | 140 | 0.04% PS80, 5 | 1.0 | 5 | Practically |
| | | X FT | | | free from |
| | | | | | visible |
| | | | | | particles |
| 9 | 140 | 0.05% PS80, 5 | 1.0 | 14 | Practically |
| | | X FT | | | free from |
| | | | | | visible |
| | | | | | particles |
| 10 | 140 | 0.07% PS80, 5 | 1.0 | 9 | Practically |
| | | X FT | | | free from |
| | | | | | visible |
| | | | | | particles |

### Example 7

### hIL4R Antibody Conformational Stability and Aggregation Assessment

Anti-hIL-4Rα antibody was prepared at 149±4.6 mg/ml in 25 mM Histidine/Histidine-HCL, 190 mM Arginine-HCL, 0.02% PS80 at varying pHs (5.5-6.5). The Drug Product was aseptically filled into 3 cc glass vials, stoppered and sealed with an aluminum overseal. For accelerated screening, samples were placed on stability at 40° C. For longer-term stability studies in addition to the accelerated 40° C. condition, studies were also performed at 5° C. Samples were analyzed by SEC HPLC, Bioanalyzer and the vials were visually inspected for particles. In addition the initial timepoint was analyzed for osmolality, pH, HIAC, and DSC.

FIG. 7 shows increasing conformational stability (Tm1) with increasing pH (pH 6.5>pH 6>pH 5.5)

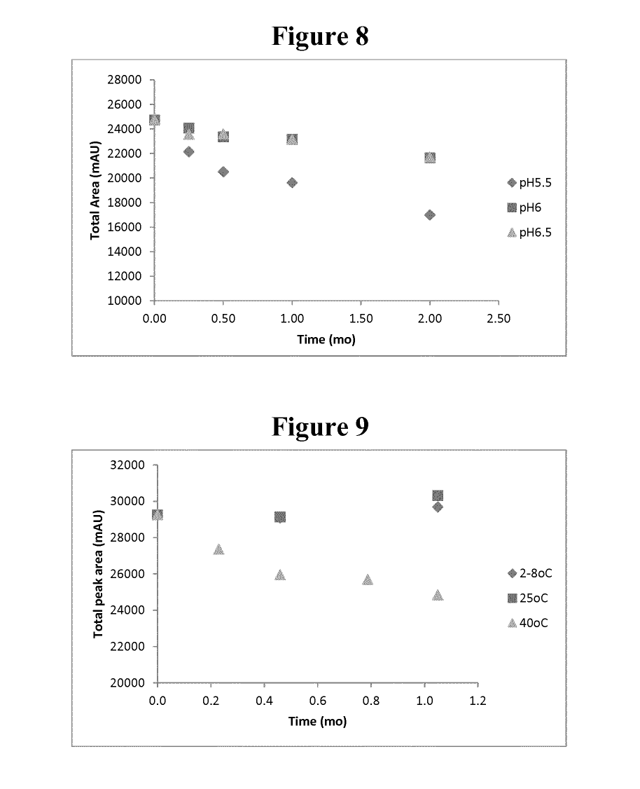

FIG. 8 shows the time dependent loss of UV absorbance of total peak area during HPSEC analysis of samples stored at 40° C. This loss of total peak area is not evident when samples have been stored for 1 month at 2-8° C. or 25° C. as shown in FIG. 9. The UV absorbance of total peak area is an indirect measure of the concentration of total protein analyzed. A loss in soluble protein is generally assumed to be due to insoluble aggregates which are not directly analyzed.

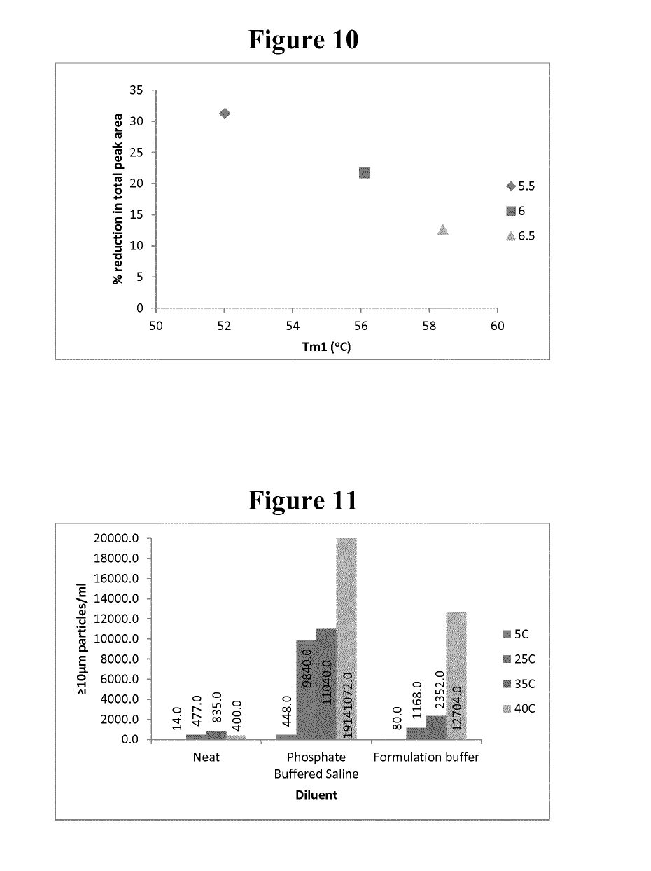

FIG. 10 shows a graph of the total percent area absorbance reduction after 8 weeks at 40° C. against the Tm1 of each formulation. These data show that the insoluble aggregate formation over time at 40° C. is dependent on the conformational stability of the molecule.

### Example 8

### Study of Impact of Diluent on hIL4R Antibody Insoluble Aggregate Formation

Previous data showed that samples stored at 40° C. for 1 month showed a time dependent loss of total peak absorbance during analysis by high performance size exclusion chromatography potentially due to the formation of insoluble aggregate before or during the analysis. Samples are diluted into phosphate buffered saline and filtered through a 0.2 μM filter prior to HPSEC analysis. The following study was to assess the impact of dilution on subvisible particle formation.

An anti-hIL-4Rα antibody formulation was made containing anti-hIL-4Rα antibody at 148.2 mg/mL in 25 mM L-histidine/L-histidine hydrochloride monohydrate, 190 mM Arginine hydrochloride, 0.04% (w/v) polysorbate 80, pH 6.0.

The Drug Product was aseptically filled into 3 cc glass vials, stoppered and sealed with an aluminum overseal. Samples were stored at 2-8° C., 25° C., 35° C. or 40° C. for 1 month. Samples were diluted 1/16 in either formulation buffer (25 mM L-histidine/L-histidine hydrochloride monohydrate, 190 mM Arginine hydrochloride, pH 6) phosphate buffered saline. Additionally samples stored at 40° C. were also diluted 1/16 into 25 mM L-histidine/L-histidine hydrochloride monohydrate, 190 mM Arginine hydrochloride, pH 7.4, 25 mM L-histidine/L-histidine hydrochloride monohydrate, 140 mM NaCl, pH 7.4 or 10 mM Phosphate, 190 mM Arginine, pH 7.4.

Diluted samples and an undiluted sample from each temperature condition were analyzed for subvisible particle number by Microflowimaging.

### Subvisible Particle Analysis

Sub-visible particles analysis was performed using flow microscopy (Brightwell Microflow Imager, MFI) using the current Formulation Sciences guidelines. Samples were analyzed neat or after dilution into phosphate buffered saline or formulation buffer. The flow cell was cleaned thoroughly with ultrapure water between each sample.

### Results and Discussion

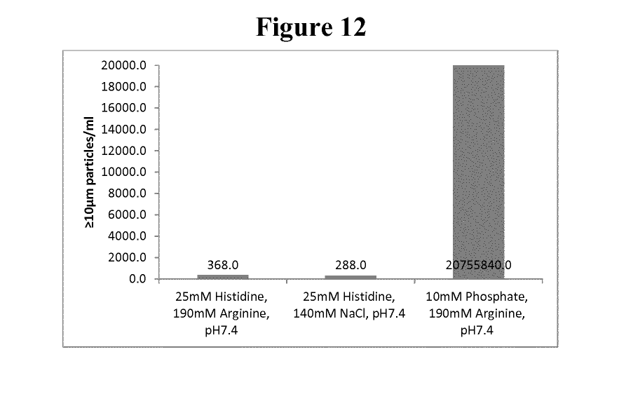

FIG. 11 shows the absolute ≧10 μm particle number taking into account the dilution factor where appropriate. Samples incubated at 40° C. for 4 weeks form high levels of ≧10 μm particles after dilution in both phosphate buffered saline and formulation buffer. Dilution of anti-hIL-4Rα antibody into phosphate buffered saline has a greater impact on the subvisible particle formation than dilution of the antibody into formulation buffer. The impact of pH, ionic strength and ion type on subvisible particle formation in thermally stressed anti-hIL-4Rα antibody was also investigated. FIG. 12 shows subvisible particle formation in thermally stressed anti-hIL-4Rα antibody is exacerbated by the presence of the phosphate ion.

The examples shown above illustrate various aspects of the invention and practice of the methods of the invention. These examples are not intended to provide an exhaustive description of the many different embodiments of the invention. Thus, although the invention has been described in some detail by way of illustration and example for purposes of clarity of understanding, those of ordinary skill in the art will realize readily that many changes and modifications can be made without departing from the spirit or scope of the appended claims.

All publications, patents and patent applications mentioned in this specification are herein incorporated by reference into the specification to the same extent as if each individual publication, patent or patent application was specifically and individually indicated to be incorporated herein by reference.

1.-30. (canceled)

31. A stable antibody formulation comprising:
(a) about 100 mg/mL to about 200 mg/mL of an antibody or fragment thereof that specifically binds human interleukin-4 receptor alpha (hIL-4Rα), wherein the antibody comprises a set of CDRs: HCDR1, HCDR2, HCDR3, LCDR1, LCDR2 and LCDR3, wherein the set of CDRs has 10 or fewer amino acid substitutions from a reference set of CDRs selected from:
(I)
HCDR1 has amino acid sequence SEQ ID NO: 193;
HCDR2 has amino acid sequence SEQ ID NO: 194;
HCDR3 has amino acid sequence SEQ ID NO: 195;
LCDR1 has amino acid sequence SEQ ID NO: 198;
LCDR2 has amino acid sequence SEQ ID NO: 199; and
LCDR3 has amino acid sequence SEQ ID NO: 200;
(II)
the HCDR1 has amino acid sequence SEQ ID NO: 363;
the HCDR2 has amino acid sequence SEQ ID NO: 364;
the HCDR3 has amino acid sequence SEQ ID NO: 365;
the LCDR1 has amino acid sequence SEQ ID NO: 368;
the LCDR2 has amino acid sequence SEQ ID NO: 369; and
the LCDR3 has amino acid sequence SEQ ID NO: 370;
OR
(III)
the HCDR1 has amino acid sequence SEQ ID NO: 233;
the HCDR2 has amino acid sequence SEQ ID NO: 234;
the HCDR3 has amino acid sequence SEQ ID NO: 235;
the LCDR1 has amino acid sequence SEQ ID NO: 238;
the LCDR2 has amino acid sequence SEQ ID NO: 239; and
the LCDR3 has amino acid sequence SEQ ID NO: 240;
(b) about 50 mM to about 400 mM of a viscosity modifier;
(c) about 0.002% to about 0.2% of a non-ionic surfactant; and
(d) a formulation buffer.

32. The antibody formulation of claim 1, wherein the antibody or fragment thereof comprises an antibody VH domain and an antibody VL domain, wherein the VH domain comprises HCDR1, HCDR2, HCDR3 and a first framework and the VL domain comprises LCDR1, LCDR2, LCDR3 and a second framework.

33. The antibody formulation of claim 1, wherein the formulation buffer is essentially free of phosphate.

34. The antibody formulation of claim 33, wherein the viscosity modifier is selected from the group consisting of histidine, arginine, lysine, polyvinyl alcohol, polyalkyl cellulose, hydroxyalkyl cellulose, glycerin, polyethylene glycol, glucose, dextrose, and sucrose.

35. The antibody formulation of claim 34, wherein the viscosity modifier is selected from the group consisting of L-arginine, L-lysine, and L-histidine.

36. The antibody formulation of claim 1, wherein the non-ionic surfactant is selected from the group consisting of Triton X-100, Tween 80, polysorbate 20, polysorbate 80, nonoxynol-9, polyoxamer, stearyl alcohol, or sorbitan monostearate.

37. The antibody formulation of claim 1, wherein the formulation buffer is an acetate buffer, TRIS buffer, HEPES buffer, hydrochloride buffer, arginine buffer, histidine buffer, glycine buffer, citrate buffer, or TES buffer.

38. The antibody formulation of claim 37, wherein the formulation buffer is an arginine buffer or histidine buffer.

39. The antibody formulation of claim 1, wherein the formulation further comprises about 100 mM to about 200 mM NaCl.

40. The antibody formulation of claim 1, wherein the formulation has a pH of about 5 to about 8.

41. The antibody formulation of claim 32, wherein the antibody or fragment thereof comprises a VH domain wherein:
a. the VH domain has amino acid sequence SEQ ID NO: 192;
b. the VH domain has amino acid sequence SEQ ID NO: 362; or
c. the VH domain has amino acid sequence SEQ ID NO: 232; and,
wherein the VH domain comprises one or more amino acid substitutions at the following residues within the framework regions, using the standard numbering of Kabat:
11, 12 in HFW1;
37, 48 in HFW2;
68, 84, 85 in HFW3; or
105, 108, 113 in HFW4.

42. The antibody formulation of claim 32, wherein the antibody or fragment thereof comprises a VL domain wherein:
a. the VL domain has amino acid sequence SEQ ID NO: 197;
b. the VL domain has amino acid sequence SEQ ID NO: 367; or
c. the VL domain has amino acid sequence SEQ ID NO: 237; and,
wherein the VL domain comprises one or more amino acid substitutions at the following residues within the framework regions, using the standard numbering of Kabat:
1, 2, 3, 9 in LFW1;
38, 42 in LFW2; or
58, 65, 66, 70, 74, 85, 87 in LFW3.

43. The antibody formulation of claim 1, wherein the antibody or fragment thereof comprises a VH and a VL domain wherein:
a. the VH domain has amino acid sequence SEQ ID NO: 192 and the VL domain has amino acid sequence SEQ ID NO: 197;
b. the VH domain has amino acid sequence SEQ ID NO: 362 and the VL domain has amino acid sequence SEQ ID NO: 367; or
c. the VH domain has amino acid sequence SEQ ID NO: 232 and the VL domain has amino acid sequence SEQ ID NO: 237; and,
wherein the VH domain and VL domain comprise one or more amino acid substitutions at the following residues within the framework regions, using the standard numbering of Kabat:
11, 12 in HFW1;
37, 48 in HFW2;
68, 84, 85 in HFW3;
105, 108, 113 in HFW4;
1, 2, 3, 9 in LFW1;
38, 42 in LFW2; or
58, 65, 66, 70, 74, 85, 87 in LFW3.

44. The antibody formulation of claim 1, wherein said formulation is stable upon storage at about 40° C. for at least 1 month; at about 25° C. for at least 3 months; or at about 5° C. for at least 18 months.

45. The antibody formulation of claim 1, wherein the antibody stored at about 40° C. for at least 1 month retains at least 80% or at least 50% of binding ability to an hIL-4Rα polypeptide compared to a reference antibody that has not been stored.

46. The antibody formulation of claim 1, wherein the antibody stored at about 5° C. for at least 6 month retains at least 80% or at least 50% of binding ability to an hIL-4Rα polypeptide compared to a reference antibody that has not been stored.

47. The antibody formulation of claim 1, wherein the formulation has a viscosity of less than 20 cP at 23° C.

48. A stable antibody formulation comprising:
(a) about 100 mg/mL to about 200 mg/mL of an antibody or fragment thereof that specifically binds human interleukin-4 receptor alpha (hIL-4Rα), wherein the antibody comprises a set of CDRs: HCDR1, HCDR2, HCDR3, LCDR1, LCDR2 and LCDR3, wherein the set of CDRs has 10 or fewer amino acid substitutions from a reference set of CDRs selected from:
(I)
HCDR1 has amino acid sequence SEQ ID NO: 193;
HCDR2 has amino acid sequence SEQ ID NO: 194;
HCDR3 has amino acid sequence SEQ ID NO: 195;
LCDR1 has amino acid sequence SEQ ID NO: 198;
LCDR2 has amino acid sequence SEQ ID NO: 199; and
LCDR3 has amino acid sequence SEQ ID NO: 200;
(II)
the HCDR1 has amino acid sequence SEQ ID NO: 363;
the HCDR2 has amino acid sequence SEQ ID NO: 364;
the HCDR3 has amino acid sequence SEQ ID NO: 365;
the LCDR1 has amino acid sequence SEQ ID NO: 368;
the LCDR2 has amino acid sequence SEQ ID NO: 369; and
the LCDR3 has amino acid sequence SEQ ID NO: 370;
OR
(III)
the HCDR1 has amino acid sequence SEQ ID NO: 233;
the HCDR2 has amino acid sequence SEQ ID NO: 234;
the HCDR3 has amino acid sequence SEQ ID NO: 235;
the LCDR1 has amino acid sequence SEQ ID NO: 238;
the LCDR2 has amino acid sequence SEQ ID NO: 239; and
the LCDR3 has amino acid sequence SEQ ID NO: 240;
(b) about 50 mM to about 400 mM arginine;
(c) about 0.002% to about 0.2% of polysorbate 80; and
(d) about 10 to about 40 mM L-histidine/L-histidine hydrochloride.

49. A pharmaceutical unit dosage form suitable for parenteral administration to a human which comprises the antibody formulation of claim 1 in a suitable container.

50. A method for treating a pulmonary disease or disorder or a chronic inflammatory skin disease or disorder in a subject, the method comprising administering a therapeutically effective amount of the antibody formulation of claim 1.


SEQUENCE LISTING (461 sequences)

SEQ ID NO: 1 - DNA, 348 bp, Homo sapiens
  Antibody 1
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggcttgactactggggcaaaggcaccctggtcaccgtctcgagt

SEQ ID NO: 2 - protein, 116 aa, Homo sapiens
  Antibody 1
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 3 - protein, 5 aa, Homo sapiens
  Antibody 1
SYYMH

SEQ ID NO: 4 - protein, 17 aa, Homo sapiens
  Antibody 1
IINPSGGSTSYAQKFQG

SEQ ID NO: 5 - protein, 7 aa, Homo sapiens
  Antibody 1
GKWWLDY

SEQ ID NO: 6 - DNA, 333 bp, Homo sapiens
  Antibody 1
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcctgagtgccaattatgtcttcggaactgggaccaagctgaccgtccta

SEQ ID NO: 7 - protein, 111 aa, Homo sapiens
  Antibody 1
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 8 - protein, 13 aa, Homo sapiens
  Antibody 1
SGGSSNIGNSYVS

SEQ ID NO: 9 - protein, 7 aa, Homo sapiens
  Antibody 1
DNNKRPS

SEQ ID NO: 10 - protein, 12 aa, Homo sapiens
  Antibody 1
GTWDTSLSANYV

SEQ ID NO: 11 - DNA, 348 bp, Homo sapiens
  Antibody 2
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggctgtacaactggggcaaaggcaccctggtcaccgtctcgagt

SEQ ID NO: 12 - protein, 116 aa, Homo sapiens
  Antibody 2
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 13 - protein, 5 aa, Homo sapiens
  Antibody 2
SYYMH

SEQ ID NO: 14 - protein, 17 aa, Homo sapiens
  Antibody 2
IINPSGGSTSYAQKFQG

SEQ ID NO: 15 - protein, 7 aa, Homo sapiens
  Antibody 2
GKWWLYN

SEQ ID NO: 16 - DNA, 333 bp, Homo sapiens
  Antibody 2
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagccagcccccgaaccccctcttcggaactgggaccaagctgaccgtccta

SEQ ID NO: 17 - protein, 111 aa, Homo sapiens
  Antibody 2
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 18 - protein, 13 aa, Homo sapiens
  Antibody 2
SGGSSNIGNSYVS

SEQ ID NO: 19 - protein, 7 aa, Homo sapiens
  Antibody 2
DNNKRPS

SEQ ID NO: 20 - protein, 12 aa, Homo sapiens
  Antibody 2
GTWDTSQPPNPL

SEQ ID NO: 21 - DNA, 348 bp, Homo sapiens
  Antibody 3
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagttgttgaagaacccctggggcaaaggcaccctggtcaccgtctcgagt

SEQ ID NO: 22 - protein, 116 aa, Homo sapiens
  Antibody 3
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 23 - protein, 5 aa, Homo sapiens
  Antibody 3
SYYMH

SEQ ID NO: 24 - protein, 17 aa, Homo sapiens
  Antibody 3
IINPSGGSTSYAQKFQG

SEQ ID NO: 25 - protein, 7 aa, Homo sapiens
  Antibody 3
GKLLKNP

SEQ ID NO: 26 - DNA, 333 bp, Homo sapiens
  Antibody 3
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatggttcggcacccccgcgagcaattatgtcttcggaactgggaccaagctgaccgtccta

SEQ ID NO: 27 - protein, 111 aa, Homo sapiens
  Antibody 3
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 28 - protein, 13 aa, Homo sapiens
  Antibody 3
SGGSSNIGNSYVS

SEQ ID NO: 29 - protein, 7 aa, Homo sapiens
  Antibody 3
DNNKRPS

SEQ ID NO: 30 - protein, 12 aa, Homo sapiens
  Antibody 3
GTWFGTPASNYV

SEQ ID NO: 31 - DNA, 348 bp, Homo sapiens; identical to SEQ ID NO: 11
  Antibody 4

SEQ ID NO: 32 - protein, 116 aa, Homo sapiens; identical to SEQ ID NO: 12
  Antibody 4

SEQ ID NO: 33 - protein, 5 aa, Homo sapiens
  Antibody 4
SYYMH

SEQ ID NO: 34 - protein, 17 aa, Homo sapiens
  Antibody 4
IINPSGGSTSYAQKFQG

SEQ ID NO: 35 - protein, 7 aa, Homo sapiens
  Antibody 4
GKWWLYN

SEQ ID NO: 36 - DNA, 333 bp, Homo sapiens
  Antibody 4
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcagccccccccagccgatcttcggaactgggaccaagctgaccgtccta

SEQ ID NO: 37 - protein, 111 aa, Homo sapiens
  Antibody 4
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 38 - protein, 13 aa, Homo sapiens
  Antibody 4
SGGSSNIGNSYVS

SEQ ID NO: 39 - protein, 7 aa, Homo sapiens
  Antibody 4
DNNKRPS

SEQ ID NO: 40 - protein, 12 aa, Homo sapiens
  Antibody 4
GTWDTSSPPQPI

SEQ ID NO: 41 - DNA, 348 bp, Homo sapiens
  Antibody 5
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggttgtacgactggggcaaaggcaccctggtcaccgtctcgagt

SEQ ID NO: 42 - protein, 116 aa, Homo sapiens
  Antibody 5
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 43 - protein, 5 aa, Homo sapiens
  Antibody 5
SYYMH

SEQ ID NO: 44 - protein, 17 aa, Homo sapiens
  Antibody 5
IINPSGGSTSYAQKFQG

SEQ ID NO: 45 - protein, 7 aa, Homo sapiens
  Antibody 5
GKWWLYD

SEQ ID NO: 46 - DNA, 333 bp, Homo sapiens; identical to SEQ ID NO: 36
  Antibody 5

SEQ ID NO: 47 - protein, 111 aa, Homo sapiens; identical to SEQ ID NO: 37
  Antibody 5

SEQ ID NO: 48 - protein, 13 aa, Homo sapiens
  Antibody 5
SGGSSNIGNSYVS

SEQ ID NO: 49 - protein, 7 aa, Homo sapiens
  Antibody 5
DNNKRPS

SEQ ID NO: 50 - protein, 12 aa, Homo sapiens
  Antibody 5
GTWDTSSPPQPI

SEQ ID NO: 51 - DNA, 348 bp, Homo sapiens
  Antibody 6
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 52 - protein, 116 aa, Homo sapiens
  Antibody 6
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 53 - protein, 5 aa, Homo sapiens
  Antibody 6
SYYMH

SEQ ID NO: 54 - protein, 17 aa, Homo sapiens
  Antibody 6
IINPSGGSTSYAQKFQG

SEQ ID NO: 55 - protein, 7 aa, Homo sapiens
  Antibody 6
GKYWMYD

SEQ ID NO: 56 - DNA, 333 bp, Homo sapiens
  Antibody 6
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacgacctaccaccccatcttcggaactgggaccaagctgaccgtccta

SEQ ID NO: 57 - protein, 111 aa, Homo sapiens
  Antibody 6
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 58 - protein, 13 aa, Homo sapiens
  Antibody 6
SGGSSNIGNSYVS

SEQ ID NO: 59 - protein, 7 aa, Homo sapiens
  Antibody 6
DNNKRPS

SEQ ID NO: 60 - protein, 12 aa, Homo sapiens
  Antibody 6
GTWDTSTTYHPI

SEQ ID NO: 61 - DNA, 348 bp, Homo sapiens
  Antibody 7
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggtggcagtactggggcaaaggcaccctggtcaccgtctcgagt

SEQ ID NO: 62 - protein, 116 aa, Homo sapiens
  Antibody 7
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 63 - protein, 5 aa, Homo sapiens
  Antibody 7
SYYMH

SEQ ID NO: 64 - protein, 17 aa, Homo sapiens
  Antibody 7
IINPSGGSTSYAQKFQG

SEQ ID NO: 65 - protein, 7 aa, Homo sapiens
  Antibody 7
GKWWWQY

SEQ ID NO: 66 - DNA, 333 bp, Homo sapiens; identical to SEQ ID NO: 36
  Antibody 7

SEQ ID NO: 67 - protein, 111 aa, Homo sapiens; identical to SEQ ID NO: 37
  Antibody 7

SEQ ID NO: 68 - protein, 13 aa, Homo sapiens
  Antibody 7
SGGSSNIGNSYVS

SEQ ID NO: 69 - protein, 7 aa, Homo sapiens
  Antibody 7
DNNKRPS

SEQ ID NO: 70 - protein, 12 aa, Homo sapiens
  Antibody 7
GTWDTSSPPQPI

SEQ ID NO: 71 - DNA, 348 bp, Homo sapiens; identical to SEQ ID NO: 61
  Antibody 8

SEQ ID NO: 72 - protein, 116 aa, Homo sapiens; identical to SEQ ID NO: 62
  Antibody 8

SEQ ID NO: 73 - protein, 5 aa, Homo sapiens
  Antibody 8
SYYMH

SEQ ID NO: 74 - protein, 17 aa, Homo sapiens
  Antibody 8
IINPSGGSTSYAQKFQG

SEQ ID NO: 75 - protein, 7 aa, Homo sapiens
  Antibody 8
GKWWWQY

SEQ ID NO: 76 - DNA, 333 bp, Homo sapiens; identical to SEQ ID NO: 56
  Antibody 8

SEQ ID NO: 77 - protein, 111 aa, Homo sapiens; identical to SEQ ID NO: 57
  Antibody 8

SEQ ID NO: 78 - protein, 13 aa, Homo sapiens
  Antibody 8
SGGSSNIGNSYVS

SEQ ID NO: 79 - protein, 7 aa, Homo sapiens
  Antibody 8
DNNKRPS

SEQ ID NO: 80 - protein, 12 aa, Homo sapiens
  Antibody 8
GTWDTSTTYHPI

SEQ ID NO: 81 - DNA, 348 bp, Homo sapiens; identical to SEQ ID NO: 11
  Antibody 9

SEQ ID NO: 82 - protein, 116 aa, Homo sapiens; identical to SEQ ID NO: 12
  Antibody 9

SEQ ID NO: 83 - protein, 5 aa, Homo sapiens
  Antibody 9
SYYMH

SEQ ID NO: 84 - protein, 17 aa, Homo sapiens
  Antibody 9
IINPSGGSTSYAQKFQG

SEQ ID NO: 85 - protein, 7 aa, Homo sapiens
  Antibody 9
GKWWLYN

SEQ ID NO: 86 - DNA, 333 bp, Homo sapiens
  Antibody 9
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacgacgatgtacccgttgttcggaactgggaccaagctgaccgtccta

SEQ ID NO: 87 - protein, 111 aa, Homo sapiens
  Antibody 9
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 88 - protein, 13 aa, Homo sapiens
  Antibody 9
SGGSSNIGNSYVS

SEQ ID NO: 89 - protein, 7 aa, Homo sapiens
  Antibody 9
DNNKRPS

SEQ ID NO: 90 - protein, 12 aa, Homo sapiens
  Antibody 9
GTWDTSTTMYPL

SEQ ID NO: 91 - DNA, 348 bp, Homo sapiens
  Antibody 10
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggctctacgactggggcaaaggcaccctggtcaccgtctcgagt

SEQ ID NO: 92 - protein, 116 aa, Homo sapiens; identical to SEQ ID NO: 42
  Antibody 10

SEQ ID NO: 93 - protein, 5 aa, Homo sapiens
  Antibody 10
SYYMH

SEQ ID NO: 94 - protein, 17 aa, Homo sapiens
  Antibody 10
IINPSGGSTSYAQKFQG

SEQ ID NO: 95 - protein, 7 aa, Homo sapiens
  Antibody 10
GKWWLYD

SEQ ID NO: 96 - DNA, 333 bp, Homo sapiens
  Antibody 10
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcaccgtcctcacccccatcttcggaactgggaccaagctgaccgtccta

SEQ ID NO: 97 - protein, 111 aa, Homo sapiens
  Antibody 10
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 98 - protein, 13 aa, Homo sapiens
  Antibody 10
SGGSSNIGNSYVS

SEQ ID NO: 99 - protein, 7 aa, Homo sapiens
  Antibody 10
DNNKRPS

SEQ ID NO: 100 - protein, 12 aa, Homo sapiens
  Antibody 10
GTWDTSTVLTPI

SEQ ID NO: 101 - DNA, 348 bp, Homo sapiens
  Antibody 11
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggttctacgactggggcaaaggcaccctggtcaccgtctcgagt

SEQ ID NO: 102 - protein, 116 aa, Homo sapiens
  Antibody 11
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 103 - protein, 5 aa, Homo sapiens
  Antibody 11
SYYMH

SEQ ID NO: 104 - protein, 17 aa, Homo sapiens
  Antibody 11
IINPSGGSTSYAQKFQG

SEQ ID NO: 105 - protein, 7 aa, Homo sapiens
  Antibody 11
GKWWFYD

SEQ ID NO: 106 - DNA, 333 bp, Homo sapiens
  Antibody 11
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagccccagcatgatcccgctcttcggaactgggaccaagctgaccgtccta

SEQ ID NO: 107 - protein, 111 aa, Homo sapiens
  Antibody 11
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 108 - protein, 13 aa, Homo sapiens
  Antibody 11
SGGSSNIGNSYVS

SEQ ID NO: 109 - protein, 7 aa, Homo sapiens
  Antibody 11
DNNKRPS

SEQ ID NO: 110 - protein, 12 aa, Homo sapiens
  Antibody 11
GTWDTSPSMIPL

SEQ ID NO: 111 - DNA, 348 bp, Homo sapiens; identical to SEQ ID NO: 101
  Antibody 12

SEQ ID NO: 112 - protein, 116 aa, Homo sapiens; identical to SEQ ID NO: 102
  Antibody 12

SEQ ID NO: 113 - protein, 5 aa, Homo sapiens
  Antibody 12
SYYMH

SEQ ID NO: 114 - protein, 17 aa, Homo sapiens
  Antibody 12
IINPSGGSTSYAQKFQG

SEQ ID NO: 115 - protein, 7 aa, Homo sapiens
  Antibody 12
GKWWFYD

SEQ ID NO: 116 - DNA, 333 bp, Homo sapiens; identical to SEQ ID NO: 86
  Antibody 12

SEQ ID NO: 117 - protein, 111 aa, Homo sapiens; identical to SEQ ID NO: 87
  Antibody 12

SEQ ID NO: 118 - protein, 13 aa, Homo sapiens
  Antibody 12
SGGSSNIGNSYVS

SEQ ID NO: 119 - protein, 7 aa, Homo sapiens
  Antibody 12
DNNKRPS

SEQ ID NO: 120 - protein, 12 aa, Homo sapiens
  Antibody 12
GTWDTSTTMYPL

SEQ ID NO: 121 - DNA, 348 bp, Homo sapiens
  Antibody 13
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagtggtggctctacgactggggcaaaggcaccctggtcaccgtctcgagt

SEQ ID NO: 122 - protein, 116 aa, Homo sapiens; identical to SEQ ID NO: 42
  Antibody 13

SEQ ID NO: 123 - protein, 5 aa, Homo sapiens
  Antibody 13
SYYMH

SEQ ID NO: 124 - protein, 17 aa, Homo sapiens
  Antibody 13
IINPSGGSTSYAQKFQG

SEQ ID NO: 125 - protein, 7 aa, Homo sapiens
  Antibody 13
GKWWLYD

SEQ ID NO: 126 - DNA, 333 bp, Homo sapiens
  Antibody 13
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacgaccttgcagccgctgttcggaactgggaccaagctgaccgtccta

SEQ ID NO: 127 - protein, 111 aa, Homo sapiens
  Antibody 13
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 128 - protein, 13 aa, Homo sapiens
  Antibody 13
SGGSSNIGNSYVS

SEQ ID NO: 129 - protein, 7 aa, Homo sapiens
  Antibody 13
DNNKRPS

SEQ ID NO: 130 - protein, 12 aa, Homo sapiens
  Antibody 13
GTWDTSTTLQPL

SEQ ID NO: 131 - DNA, 348 bp, Homo sapiens; identical to SEQ ID NO: 11
  Antibody 14

SEQ ID NO: 132 - protein, 116 aa, Homo sapiens; identical to SEQ ID NO: 12
  Antibody 14

SEQ ID NO: 133 - protein, 5 aa, Homo sapiens
  Antibody 14
SYYMH

SEQ ID NO: 134 - protein, 17 aa, Homo sapiens
  Antibody 14
IINPSGGSTSYAQKFQG

SEQ ID NO: 135 - protein, 7 aa, Homo sapiens
  Antibody 14
GKWWLYN

SEQ ID NO: 136 - DNA, 333 bp, Homo sapiens
  Antibody 14
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagccccccgaccaagcccttgttcggaactgggaccaagctgaccgtccta

SEQ ID NO: 137 - protein, 111 aa, Homo sapiens
  Antibody 14
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 138 - protein, 13 aa, Homo sapiens
  Antibody 14
SGGSSNIGNSYVS

SEQ ID NO: 139 - protein, 7 aa, Homo sapiens
  Antibody 14
DNNKRPS

SEQ ID NO: 140 - protein, 12 aa, Homo sapiens
  Antibody 14
GTWDTSPPTKPL

SEQ ID NO: 141 - DNA, 348 bp, Homo sapiens; identical to SEQ ID NO: 11
  Antibody 15

SEQ ID NO: 142 - protein, 116 aa, Homo sapiens; identical to SEQ ID NO: 12
  Antibody 15

SEQ ID NO: 143 - protein, 5 aa, Homo sapiens
  Antibody 15
SYYMH

SEQ ID NO: 144 - protein, 17 aa, Homo sapiens
  Antibody 15
IINPSGGSTSYAQKFQG

SEQ ID NO: 145 - protein, 7 aa, Homo sapiens
  Antibody 15
GKWWLYN

SEQ ID NO: 146 - DNA, 333 bp, Homo sapiens
  Antibody 15
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacccaccggcatcccctcttcggaactgggaccaagctgaccgtccta

SEQ ID NO: 147 - protein, 111 aa, Homo sapiens
  Antibody 15
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 148 - protein, 13 aa, Homo sapiens
  Antibody 15
SGGSSNIGNSYVS

SEQ ID NO: 149 - protein, 7 aa, Homo sapiens
  Antibody 15
DNNKRPS

SEQ ID NO: 150 - protein, 12 aa, Homo sapiens
  Antibody 15
GTWDTSTHRHPL

SEQ ID NO: 151 - DNA, 348 bp, Homo sapiens; identical to SEQ ID NO: 11
  Antibody 16

SEQ ID NO: 152 - protein, 116 aa, Homo sapiens; identical to SEQ ID NO: 12
  Antibody 16

SEQ ID NO: 153 - protein, 5 aa, Homo sapiens
  Antibody 16
SYYMH

SEQ ID NO: 154 - protein, 17 aa, Homo sapiens
  Antibody 16
IINPSGGSTSYAQKFQG

SEQ ID NO: 155 - protein, 7 aa, Homo sapiens
  Antibody 16
GKWWLYN

SEQ ID NO: 156 - DNA, 333 bp, Homo sapiens; identical to SEQ ID NO: 56
  Antibody 16

SEQ ID NO: 157 - protein, 111 aa, Homo sapiens; identical to SEQ ID NO: 57
  Antibody 16

SEQ ID NO: 158 - protein, 13 aa, Homo sapiens
  Antibody 16
SGGSSNIGNSYVS

SEQ ID NO: 159 - protein, 7 aa, Homo sapiens
  Antibody 16
DNNKRPS

SEQ ID NO: 160 - protein, 12 aa, Homo sapiens
  Antibody 16
GTWDTSTTYHPI

SEQ ID NO: 161 - DNA, 348 bp, Homo sapiens
  Antibody 17
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaaatggtggtggcagcactggggcaaaggcaccctggtcaccgtctcgagt

SEQ ID NO: 162 - protein, 116 aa, Homo sapiens
  Antibody 17
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 163 - protein, 5 aa, Homo sapiens
  Antibody 17
SYYMH

SEQ ID NO: 164 - protein, 17 aa, Homo sapiens
  Antibody 17
IINPSGGSTSYAQKFQG

SEQ ID NO: 165 - protein, 7 aa, Homo sapiens
  Antibody 17
GKWWWQH

SEQ ID NO: 166 - DNA, 333 bp, Homo sapiens
  Antibody 17
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcccggtggacaggccgatcttcggaactgggaccaagctgaccgtccta

SEQ ID NO: 167 - protein, 111 aa, Homo sapiens
  Antibody 17
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 168 - protein, 13 aa, Homo sapiens
  Antibody 17
SGGSSNIGNSYVS

SEQ ID NO: 169 - protein, 7 aa, Homo sapiens
  Antibody 17
DNNKRPS

SEQ ID NO: 170 - protein, 12 aa, Homo sapiens
  Antibody 17
GTWDTSPVDRPI

SEQ ID NO: 171 - DNA, 348 bp, Homo sapiens; identical to SEQ ID NO: 161
  Antibody 18

SEQ ID NO: 172 - protein, 116 aa, Homo sapiens; identical to SEQ ID NO: 162
  Antibody 18

SEQ ID NO: 173 - protein, 5 aa, Homo sapiens
  Antibody 18
SYYMH

SEQ ID NO: 174 - protein, 17 aa, Homo sapiens
  Antibody 18
IINPSGGSTSYAQKFQG

SEQ ID NO: 175 - protein, 7 aa, Homo sapiens
  Antibody 18
GKWWWQH

SEQ ID NO: 176 - DNA, 333 bp, Homo sapiens
  Antibody 18
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcaccaccccgatgcccgtcttcggaactgggaccaagctgaccgtccta

SEQ ID NO: 177 - protein, 111 aa, Homo sapiens
  Antibody 18
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 178 - protein, 13 aa, Homo sapiens
  Antibody 18
SGGSSNIGNSYVS

SEQ ID NO: 179 - protein, 7 aa, Homo sapiens
  Antibody 18
DNNKRPS

SEQ ID NO: 180 - protein, 12 aa, Homo sapiens
  Antibody 18
GTWDTSTTPMPV

SEQ ID NO: 181 - DNA, 348 bp, Homo sapiens; identical to SEQ ID NO: 161
  Antibody 19

SEQ ID NO: 182 - protein, 116 aa, Homo sapiens; identical to SEQ ID NO: 162
  Antibody 19

SEQ ID NO: 183 - protein, 5 aa, Homo sapiens
  Antibody 19
SYYMH

SEQ ID NO: 184 - protein, 17 aa, Homo sapiens
  Antibody 19
IINPSGGSTSYAQKFQG

SEQ ID NO: 185 - protein, 7 aa, Homo sapiens
  Antibody 19
GKWWWQH

SEQ ID NO: 186 - DNA, 333 bp, Homo sapiens; identical to SEQ ID NO: 56
  Antibody 19

SEQ ID NO: 187 - protein, 111 aa, Homo sapiens; identical to SEQ ID NO: 57
  Antibody 19

SEQ ID NO: 188 - protein, 13 aa, Homo sapiens
  Antibody 19
SGGSSNIGNSYVS

SEQ ID NO: 189 - protein, 7 aa, Homo sapiens
  Antibody 19
DNNKRPS

SEQ ID NO: 190 - protein, 12 aa, Homo sapiens
  Antibody 19
GTWDTSTTYHPI

SEQ ID NO: 191 - DNA, 348 bp, Homo sapiens; identical to SEQ ID NO: 51
  Antibody 20

SEQ ID NO: 192 - protein, 116 aa, Homo sapiens; identical to SEQ ID NO: 52
  Antibody 20

SEQ ID NO: 193 - protein, 5 aa, Homo sapiens
  Antibody 20
SYYMH

SEQ ID NO: 194 - protein, 17 aa, Homo sapiens
  Antibody 20
IINPSGGSTSYAQKFQG

SEQ ID NO: 195 - protein, 7 aa, Homo sapiens
  Antibody 20
GKYWMYD

SEQ ID NO: 196 - DNA, 333 bp, Homo sapiens
  Antibody 20
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacggtgtgggagtggccgttcggaactgggaccaagctgaccgtccta

SEQ ID NO: 197 - protein, 111 aa, Homo sapiens
  Antibody 20
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 198 - protein, 13 aa, Homo sapiens
  Antibody 20
SGGSSNIGNSYVS

SEQ ID NO: 199 - protein, 7 aa, Homo sapiens
  Antibody 20
DNNKRPS

SEQ ID NO: 200 - protein, 12 aa, Homo sapiens
  Antibody 20
GTWDTSTVWEWP

SEQ ID NO: 201 - DNA, 348 bp, Homo sapiens
  Antibody 21
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatggggataatcaaccctagtggtggtagcgcaagctacgcgcagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 202 - protein, 116 aa, Homo sapiens
  Antibody 21
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 203 - protein, 5 aa, Homo sapiens
  Antibody 21
SYYMH

SEQ ID NO: 204 - protein, 17 aa, Homo sapiens
  Antibody 21
IINPSGGSASYAQKFQG

SEQ ID NO: 205 - protein, 7 aa, Homo sapiens
  Antibody 21
GKYWMYD

SEQ ID NO: 206 - DNA, 333 bp, Homo sapiens
  Antibody 21
cagtctgtgttgacgcagccgccctcggtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgtttatttctgcggaacatgggataccagcacggtgtgggagtggccgttcggaactgggaccaagctgaccgtccta

SEQ ID NO: 207 - protein, 111 aa, Homo sapiens
  Antibody 21
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 208 - protein, 13 aa, Homo sapiens
  Antibody 21
SGGSSNIGNSYVS

SEQ ID NO: 209 - protein, 7 aa, Homo sapiens
  Antibody 21
DNNKRPS

SEQ ID NO: 210 - protein, 12 aa, Homo sapiens
  Antibody 21
GTWDTSTVWEWP

SEQ ID NO: 211 - DNA, 348 bp, Homo sapiens
  Antibody 22
caggtccagctggtgcagtctggggctgaggtgaagaagcccggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 212 - protein, 116 aa, Homo sapiens; identical to SEQ ID NO: 52
  Antibody 22

SEQ ID NO: 213 - protein, 5 aa, Homo sapiens
  Antibody 22
SYYMH

SEQ ID NO: 214 - protein, 17 aa, Homo sapiens
  Antibody 22
IINPSGGSTSYAQKFQG

SEQ ID NO: 215 - protein, 7 aa, Homo sapiens
  Antibody 22
GKYWMYD

SEQ ID NO: 216 - DNA, 333 bp, Homo sapiens
  Antibody 22
cagcccgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattatttctgcggaacatgggataccagcacggtgtgggagtggccgttcggaactgggaccaagctgaccgtccta

SEQ ID NO: 217 - protein, 111 aa, Homo sapiens
  Antibody 22
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 218 - protein, 13 aa, Homo sapiens
  Antibody 22
SGGSSNIGNSYVS

SEQ ID NO: 219 - protein, 7 aa, Homo sapiens
  Antibody 22
DNNKRPS

SEQ ID NO: 220 - protein, 12 aa, Homo sapiens
  Antibody 22
GTWDTSTVWEWP

SEQ ID NO: 221 - DNA, 348 bp, Homo sapiens
  Antibody 23
caggtccagctggtgcagtctggggctgaggtgaggaagcctggggcctcagtgaaggtttcctgcaaggcatctggatacgccttcaccagctactatatgcactgggcgcgacaggcccccgggcaagggcttgagtggatgggaataatcaaccctagtggtggtagcacaagctacgcgcagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 222 - protein, 116 aa, Homo sapiens
  Antibody 23
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 223 - protein, 5 aa, Homo sapiens
  Antibody 23
SYYMH

SEQ ID NO: 224 - protein, 17 aa, Homo sapiens
  Antibody 23
IINPSGGSTSYAQKFQG

SEQ ID NO: 225 - protein, 7 aa, Homo sapiens
  Antibody 23
GKYWMYD

SEQ ID NO: 226 - DNA, 333 bp, Homo sapiens
  Antibody 23
cagtctgtgttgacgcagccgccctcagtgtctgcggccccaggacagaaggtcaccatctcctgctctggaggcagctccaacattgggaataattatgtatcctggtaccagcaactcccaggaacagcccccaaactcctcatttacgacaataataagcgacccccagggattcctgaccgattctctggctccaagtctggcacgtcagccaccctggccatcaccggactccagactggggacgaggccgattattactgcggaacatgggataccagcacggtgtgggagtggccgttcggaactgggaccaagctgaccgtccta

SEQ ID NO: 227 - protein, 111 aa, Homo sapiens
  Antibody 23
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 228 - protein, 13 aa, Homo sapiens
  Antibody 23
SGGSSNIGNNYVS

SEQ ID NO: 229 - protein, 7 aa, Homo sapiens
  Antibody 23
DNNKRPP

SEQ ID NO: 230 - protein, 12 aa, Homo sapiens
  Antibody 23
GTWDTSTVWEWP

SEQ ID NO: 231 - DNA, 348 bp, Homo sapiens
  Antibody 24
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagaggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 232 - protein, 116 aa, Homo sapiens
  Antibody 24
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 233 - protein, 5 aa, Homo sapiens
  Antibody 24
SYYMH

SEQ ID NO: 234 - protein, 17 aa, Homo sapiens
  Antibody 24
IINPRGGSTSYAQKFQG

SEQ ID NO: 235 - protein, 7 aa, Homo sapiens
  Antibody 24
GKYWMYD

SEQ ID NO: 236 - DNA, 333 bp, Homo sapiens
  Antibody 24
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattatttctgcggaacatgggataccagcacggtgtgggagtggccgttcggaactgggaccaagctgaccgtccta

SEQ ID NO: 237 - protein, 111 aa, Homo sapiens
  Antibody 24
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 238 - protein, 13 aa, Homo sapiens
  Antibody 24
SGGSSNIGNSYVS

SEQ ID NO: 239 - protein, 7 aa, Homo sapiens
  Antibody 24
DNNKRPS

SEQ ID NO: 240 - protein, 12 aa, Homo sapiens
  Antibody 24
GTWDTSTVWEWP

SEQ ID NO: 241 - DNA, 348 bp, Homo sapiens
  Antibody 25
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagaggtggtagcgcaagctacgcacagaagttccagggcagagtctccatgaccagggacacgtccacgagcacagtctacatggagctgagcagcctgaggtctgaggacacggccgtgtattactgtgcgagaggaaagtactggatgtacgactggggcaaaggcaccctggtcaccgtctcgagt

SEQ ID NO: 242 - protein, 116 aa, Homo sapiens
  Antibody 25
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 243 - protein, 5 aa, Homo sapiens
  Antibody 25
SYYMH

SEQ ID NO: 244 - protein, 17 aa, Homo sapiens
  Antibody 25
IINPRGGSASYAQKFQG

SEQ ID NO: 245 - protein, 7 aa, Homo sapiens
  Antibody 25
GKYWMYD

SEQ ID NO: 246 - DNA, 333 bp, Homo sapiens
  Antibody 25
cagtctgtgttgacgcagccgccctcagtgtctgcggccccgggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgacagccaccctggccatcaccggactccagactggggacgaggccgattattactgcggaacatgggttaccagcacggtgtgggagtggccgttcggaactgggaccaagctgaccgtccta

SEQ ID NO: 247 - protein, 111 aa, Homo sapiens
  Antibody 25
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 248 - protein, 13 aa, Homo sapiens
  Antibody 25
SGGSSNIGNSYVS

SEQ ID NO: 249 - protein, 7 aa, Homo sapiens
  Antibody 25
DNNKRPS

SEQ ID NO: 250 - protein, 12 aa, Homo sapiens
  Antibody 25
GTWVTSTVWEWP

SEQ ID NO: 251 - DNA, 348 bp, Homo sapiens; identical to SEQ ID NO: 51
  Antibody 26

SEQ ID NO: 252 - protein, 116 aa, Homo sapiens; identical to SEQ ID NO: 52
  Antibody 26

SEQ ID NO: 253 - protein, 5 aa, Homo sapiens
  Antibody 26
SYYMH

SEQ ID NO: 254 - protein, 17 aa, Homo sapiens
  Antibody 26
IINPSGGSTSYAQKFQG

SEQ ID NO: 255 - protein, 7 aa, Homo sapiens
  Antibody 26
GKYWMYD

SEQ ID NO: 256 - DNA, 333 bp, Homo sapiens; identical to SEQ ID NO: 236
  Antibody 26

SEQ ID NO: 257 - protein, 111 aa, Homo sapiens; identical to SEQ ID NO: 237
  Antibody 26

SEQ ID NO: 258 - protein, 13 aa, Homo sapiens
  Antibody 26
SGGSSNIGNSYVS

SEQ ID NO: 259 - protein, 7 aa, Homo sapiens
  Antibody 26
DNNKRPS

SEQ ID NO: 260 - protein, 12 aa, Homo sapiens
  Antibody 26
GTWDTSTVWEWP

SEQ ID NO: 261 - DNA, 348 bp, Homo sapiens
  Antibody 27
caggtccagctggtgcagtctggggctgaggtgaggaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagttaccatgaccagggacacgtccacgagcacggtctacatggagctgagcagcctgagacctgaggacacggccgtgtattactgtgcgagaggaaagtactggatgtacgactggggcaaaggcacccaggtcaccgtctcgagt

SEQ ID NO: 262 - protein, 116 aa, Homo sapiens
  Antibody 27
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 263 - protein, 5 aa, Homo sapiens
  Antibody 27
SYYMH

SEQ ID NO: 264 - protein, 17 aa, Homo sapiens
  Antibody 27
IINPSGGSTSYAQKFQG

SEQ ID NO: 265 - protein, 7 aa, Homo sapiens
  Antibody 27
GKYWMYD

SEQ ID NO: 266 - DNA, 333 bp, Homo sapiens
  Antibody 27
cagtctgtgttgacgcagccgcccttagtgtctgcggccccaggacagaaggtcaccatctcctgctctggaggcagctccaacattgggaatagttatgtatcctggtaccagcgactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacggtgtgggagtggccgttcggaactgggaccaagctgaccgtccta

SEQ ID NO: 267 - protein, 111 aa, Homo sapiens
  Antibody 27
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 268 - protein, 13 aa, Homo sapiens
  Antibody 27
SGGSSNIGNSYVS

SEQ ID NO: 269 - protein, 7 aa, Homo sapiens
  Antibody 27
DNNKRPS

SEQ ID NO: 270 - protein, 12 aa, Homo sapiens
  Antibody 27
GTWDTSTVWEWP

SEQ ID NO: 271 - DNA, 348 bp, Homo sapiens
  Antibody 28
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaagacacggccgtgtattactgtgcgagaggaaagtactggatgtacgactggggcaacggcaccctggtcaccgtctcgagt

SEQ ID NO: 272 - protein, 116 aa, Homo sapiens
  Antibody 28
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 273 - protein, 5 aa, Homo sapiens
  Antibody 28
SYYMH

SEQ ID NO: 274 - protein, 17 aa, Homo sapiens
  Antibody 28
IINPSGGSTSYAQKFQG

SEQ ID NO: 275 - protein, 7 aa, Homo sapiens
  Antibody 28
GKYWMYD

SEQ ID NO: 276 - DNA, 333 bp, Homo sapiens
  Antibody 28
ctgcctgtgttgacgcagccgccctcagtgtctgcggccccaggacagaaggtcaccatctcctgctctggaggcagctccagcattgggaatagttatgtatcctggtaccagcaactcccaggagcagcccccaaactcctcatttacgacaacaataagcgaccctcagggattcctgaccgattctctggcttcaggtctggcacgtcagccaccctggccatcaccggactccagactggggacgaggccgattattactgcggaacatgggataccagcccggtgtgggagtggccgttcggaactgggaccaagctgaccgtccta

SEQ ID NO: 277 - protein, 111 aa, Homo sapiens
  Antibody 28
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 278 - protein, 13 aa, Homo sapiens
  Antibody 28
SGGSSSIGNSYVS

SEQ ID NO: 279 - protein, 7 aa, Homo sapiens
  Antibody 28
DNNKRPS

SEQ ID NO: 280 - protein, 12 aa, Homo sapiens
  Antibody 28
GTWDTSPVWEWP

SEQ ID NO: 281 - DNA, 348 bp, Homo sapiens
  Antibody 29
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcacccgggtcaccgtctcgagt

SEQ ID NO: 282 - protein, 116 aa, Homo sapiens
  Antibody 29
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 283 - protein, 5 aa, Homo sapiens
  Antibody 29
SYYMH

SEQ ID NO: 284 - protein, 17 aa, Homo sapiens
  Antibody 29
IINPSGGSTSYAQKFQG

SEQ ID NO: 285 - protein, 7 aa, Homo sapiens
  Antibody 29
GKYWMYD

SEQ ID NO: 286 - DNA, 333 bp, Homo sapiens
  Antibody 29
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcccggtgtgggagtggccgttcggaactgggaccaagctgaccgtccta

SEQ ID NO: 287 - protein, 111 aa, Homo sapiens
  Antibody 29
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 288 - protein, 13 aa, Homo sapiens
  Antibody 29
SGGSSNIGNSYVS

SEQ ID NO: 289 - protein, 7 aa, Homo sapiens
  Antibody 29
DNNKRPS

SEQ ID NO: 290 - protein, 12 aa, Homo sapiens
  Antibody 29
GTWDTSPVWEWP

SEQ ID NO: 291 - DNA, 348 bp, Homo sapiens
  Antibody 30
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatggggataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 292 - protein, 116 aa, Homo sapiens; identical to SEQ ID NO: 52
  Antibody 30

SEQ ID NO: 293 - protein, 5 aa, Homo sapiens
  Antibody 30
SYYMH

SEQ ID NO: 294 - protein, 17 aa, Homo sapiens
  Antibody 30
IINPSGGSTSYAQKFQG

SEQ ID NO: 295 - protein, 7 aa, Homo sapiens
  Antibody 30
GKYWMYD

SEQ ID NO: 296 - DNA, 333 bp, Homo sapiens
  Antibody 30
cagtctgtgttgacgcagccgccctcagtgtctgcggccccaggacagaaggtcaccatctcctgctctgggggcagctccaacattgggaatagttatgtatcctggtaccagcgactcccaggagcagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacggtgtgggagtggccgttcggaactgggaccaagctgaccgtccta

SEQ ID NO: 297 - protein, 111 aa, Homo sapiens
  Antibody 30
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 298 - protein, 13 aa, Homo sapiens
  Antibody 30
SGGSSNIGNSYVS

SEQ ID NO: 299 - protein, 7 aa, Homo sapiens
  Antibody 30
DNNKRPS

SEQ ID NO: 300 - protein, 12 aa, Homo sapiens
  Antibody 30
GTWDTSTVWEWP

SEQ ID NO: 301 - DNA, 348 bp, Homo sapiens; identical to SEQ ID NO: 51
  Antibody 31

SEQ ID NO: 302 - protein, 116 aa, Homo sapiens; identical to SEQ ID NO: 52
  Antibody 31

SEQ ID NO: 303 - protein, 5 aa, Homo sapiens
  Antibody 31
SYYMH

SEQ ID NO: 304 - protein, 17 aa, Homo sapiens
  Antibody 31
IINPSGGSTSYAQKFQG

SEQ ID NO: 305 - protein, 7 aa, Homo sapiens
  Antibody 31
GKYWMYD

SEQ ID NO: 306 - DNA, 333 bp, Homo sapiens
  Antibody 31
cagtctgtgttgacgcagccgccctcagtgtctgcggccccaggacagaaggtcaccatctcctgctctggaggcagctccagcattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggctaccagcccggtgtgggagtggccgttcggaactgggaccaagctgaccgtccta

SEQ ID NO: 307 - protein, 111 aa, Homo sapiens
  Antibody 31
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 308 - protein, 13 aa, Homo sapiens
  Antibody 31
SGGSSSIGNSYVS

SEQ ID NO: 309 - protein, 7 aa, Homo sapiens
  Antibody 31
DNNKRPS

SEQ ID NO: 310 - protein, 12 aa, Homo sapiens
  Antibody 31
GTWATSPVWEWP

SEQ ID NO: 311 - DNA, 348 bp, Homo sapiens; identical to SEQ ID NO: 51
  Antibody 32

SEQ ID NO: 312 - protein, 116 aa, Homo sapiens; identical to SEQ ID NO: 52
  Antibody 32

SEQ ID NO: 313 - protein, 5 aa, Homo sapiens
  Antibody 32
SYYMH

SEQ ID NO: 314 - protein, 17 aa, Homo sapiens
  Antibody 32
IINPSGGSTSYAQKFQG

SEQ ID NO: 315 - protein, 7 aa, Homo sapiens
  Antibody 32
GKYWMYD

SEQ ID NO: 316 - DNA, 333 bp, Homo sapiens
  Antibody 32
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattatttctgcggaacatgggataccagcacggcgtgggagtggccgttcggaactgggaccaagctgaccgtccta

SEQ ID NO: 317 - protein, 111 aa, Homo sapiens
  Antibody 32
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 318 - protein, 13 aa, Homo sapiens
  Antibody 32
SGGSSNIGNSYVS

SEQ ID NO: 319 - protein, 7 aa, Homo sapiens
  Antibody 32
DNNKRPS

SEQ ID NO: 320 - protein, 12 aa, Homo sapiens
  Antibody 32
GTWDTSTAWEWP

SEQ ID NO: 321 - DNA, 348 bp, Homo sapiens
  Antibody 33
caggtccagctggtgcagtctggggctgaggagaagaagcctggggcctcagtgaaggtttcctgcaaggcatctggatacgccttcaccagctactatatgcactgggcgcgacaggcccctggacaagggcttgagtggatgggaataatcaaccctagtggtggtagcactagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 322 - protein, 116 aa, Homo sapiens
  Antibody 33
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 323 - protein, 5 aa, Homo sapiens
  Antibody 33
SYYMH

SEQ ID NO: 324 - protein, 17 aa, Homo sapiens
  Antibody 33
IINPSGGSTSYAQKFQG

SEQ ID NO: 325 - protein, 7 aa, Homo sapiens
  Antibody 33
GKYWMYD

SEQ ID NO: 326 - DNA, 333 bp, Homo sapiens
  Antibody 33
cagtctgcgttgacgcagccgccctcagtgtctgcggccccaggacagaaggtcaccatctcctgctctggaggcagctccaacattgggaatagttatgtatcctggtaccagcaactccctggaacagcccccaaactcctcatttacgacaataataagcgaccctcagggattcctgaccgattctctggctccaagtctggcacgtcagccaccctggccatcaccggactccagactggggacgaggccgattatttctgcggaacatgggataccagcacggtgtgggagtggccgttcggaaccgggaccaagctgaccgtccta

SEQ ID NO: 327 - protein, 111 aa, Homo sapiens
  Antibody 33
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 328 - protein, 13 aa, Homo sapiens
  Antibody 33
SGGSSNIGNSYVS

SEQ ID NO: 329 - protein, 7 aa, Homo sapiens
  Antibody 33
DNNKRPS

SEQ ID NO: 330 - protein, 12 aa, Homo sapiens
  Antibody 33
GTWDTSTVWEWP

SEQ ID NO: 331 - DNA, 348 bp, Homo sapiens
  Antibody 34
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtctccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 332 - protein, 116 aa, Homo sapiens
  Antibody 34
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 333 - protein, 5 aa, Homo sapiens
  Antibody 34
SYYMH

SEQ ID NO: 334 - protein, 17 aa, Homo sapiens
  Antibody 34
IINPSGGSTSYAQKFQG

SEQ ID NO: 335 - protein, 7 aa, Homo sapiens
  Antibody 34
GKYWMYD

SEQ ID NO: 336 - DNA, 333 bp, Homo sapiens
  Antibody 34
cagtctgtgttgacgcagccgccctcagtgtccgcggccccaggacagaaggtcaccatctcctgctctggaggcagctccaacattgggaatagttatgtgtcctggtaccagcaactcccaggaacggcccccaaactcctcatttacgacaataataagcgaccctcagggattcctgaccgattctctggctccaagtctggcacgtcagccaccctggccatcaccggactccagactggggacgaggccgattatttctgcggaacatgggataccagcacggtgtgggagtggccgttcggaactgggaccaagctgaccgtccta

SEQ ID NO: 337 - protein, 111 aa, Homo sapiens; identical to SEQ ID NO: 237
  Antibody 34

SEQ ID NO: 338 - protein, 13 aa, Homo sapiens
  Antibody 34
SGGSSNIGNSYVS

SEQ ID NO: 339 - protein, 7 aa, Homo sapiens
  Antibody 34
DNNKRPS

SEQ ID NO: 340 - protein, 12 aa, Homo sapiens
  Antibody 34
GTWDTSTVWEWP

SEQ ID NO: 341 - DNA, 348 bp, Homo sapiens; identical to SEQ ID NO: 51
  Antibody 35

SEQ ID NO: 342 - protein, 116 aa, Homo sapiens; identical to SEQ ID NO: 52
  Antibody 35

SEQ ID NO: 343 - protein, 5 aa, Homo sapiens
  Antibody 35
SYYMH

SEQ ID NO: 344 - protein, 17 aa, Homo sapiens
  Antibody 35
IINPSGGSTSYAQKFQG

SEQ ID NO: 345 - protein, 7 aa, Homo sapiens
  Antibody 35
GKYWMYD

SEQ ID NO: 346 - DNA, 333 bp, Homo sapiens; identical to SEQ ID NO: 286
  Antibody 35

SEQ ID NO: 347 - protein, 111 aa, Homo sapiens; identical to SEQ ID NO: 287
  Antibody 35

SEQ ID NO: 348 - protein, 13 aa, Homo sapiens
  Antibody 35
SGGSSNIGNSYVS

SEQ ID NO: 349 - protein, 7 aa, Homo sapiens
  Antibody 35
DNNKRPS

SEQ ID NO: 350 - protein, 12 aa, Homo sapiens
  Antibody 35
GTWDTSPVWEWP

SEQ ID NO: 351 - DNA, 348 bp, Homo sapiens
  Antibody 36
caggtccagctggtgcagtctggggctgaggtgaagaagcctggggcctcagtgaaggtttcctgcaaggcatctggatacgccttcaccagctactacatgcactgggcgcgacaggcccctggacaagggcttgagtggatgggaataatcaaccctagtggtggtagcgcaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 352 - protein, 116 aa, Homo sapiens; identical to SEQ ID NO: 202
  Antibody 36

SEQ ID NO: 353 - protein, 5 aa, Homo sapiens
  Antibody 36
SYYMH

SEQ ID NO: 354 - protein, 17 aa, Homo sapiens
  Antibody 36
IINPSGGSASYAQKFQG

SEQ ID NO: 355 - protein, 7 aa, Homo sapiens
  Antibody 36
GKYWMYD

SEQ ID NO: 356 - DNA, 333 bp, Homo sapiens
  Antibody 36
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggattccagcacggtgtgggagtggccgttcggaactgggaccaagctgaccgtccta

SEQ ID NO: 357 - protein, 111 aa, Homo sapiens
  Antibody 36
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 358 - protein, 13 aa, Homo sapiens
  Antibody 36
SGGSSNIGNSYVS

SEQ ID NO: 359 - protein, 7 aa, Homo sapiens
  Antibody 36
DNNKRPS

SEQ ID NO: 360 - protein, 12 aa, Homo sapiens
  Antibody 36
GTWDSSTVWEWP

SEQ ID NO: 361 - DNA, 348 bp, Homo sapiens
  Antibody 37
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagaggtggtagcacaagctacgcacagaagttccagggcagagtcgccatgaccagggacacgtccacgagcacagtctacatggagctgagcagcctgagacctgaggacacggccgtgtattactgtgcgagaggaaagtactggatgtacgactggggcaaaggcaccctggtcaccgtctcgagt

SEQ ID NO: 362 - protein, 116 aa, Homo sapiens
  Antibody 37
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 363 - protein, 5 aa, Homo sapiens
  Antibody 37
SYYMH

SEQ ID NO: 364 - protein, 17 aa, Homo sapiens
  Antibody 37
IINPRGGSTSYAQKFQG

SEQ ID NO: 365 - protein, 7 aa, Homo sapiens
  Antibody 37
GKYWMYD

SEQ ID NO: 366 - DNA, 333 bp, Homo sapiens
  Antibody 37
cagtctgtgttgacgcagccgccctcagtgtcagcggccccaggacagaaggtcaccatctcctgctctggaggcggctccagcattgggaatagctatgtatcctggtaccagcaactcccaggaacagcccccaaactcctcatctacgacaataataagcgaccctcaggggttcctgaccgattctctggctccaagtctggcacgtcggccaccctggccatcaccggactccagactggggacgaggccgattattactgcggaacatgggataccagcccggtgtgggagtggccgttcggaactgggaccaagctgaccgtccta

SEQ ID NO: 367 - protein, 111 aa, Homo sapiens
  Antibody 37
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 368 - protein, 13 aa, Homo sapiens
  Antibody 37
SGGGSSIGNSYVS

SEQ ID NO: 369 - protein, 7 aa, Homo sapiens
  Antibody 37
DNNKRPS

SEQ ID NO: 370 - protein, 12 aa, Homo sapiens
  Antibody 37
GTWDTSPVWEWP

SEQ ID NO: 371 - DNA, 348 bp, Homo sapiens
  Antibody 38
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctagtggtggtagcgcaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 372 - protein, 116 aa, Homo sapiens; identical to SEQ ID NO: 202
  Antibody 38

SEQ ID NO: 373 - protein, 5 aa, Homo sapiens
  Antibody 38
SYYMH

SEQ ID NO: 374 - protein, 17 aa, Homo sapiens
  Antibody 38
IINPSGGSASYAQKFQG

SEQ ID NO: 375 - protein, 7 aa, Homo sapiens
  Antibody 38
GKYWMYD

SEQ ID NO: 376 - DNA, 333 bp, Homo sapiens; identical to SEQ ID NO: 236
  Antibody 38

SEQ ID NO: 377 - protein, 111 aa, Homo sapiens; identical to SEQ ID NO: 237
  Antibody 38

SEQ ID NO: 378 - protein, 13 aa, Homo sapiens
  Antibody 38
SGGSSNIGNSYVS

SEQ ID NO: 379 - protein, 7 aa, Homo sapiens
  Antibody 38
DNNKRPS

SEQ ID NO: 380 - protein, 12 aa, Homo sapiens
  Antibody 38
GTWDTSTVWEWP

SEQ ID NO: 381 - DNA, 348 bp, Homo sapiens
  Antibody 39
caggtccagctggtgcagtctggggctgaggtgaagaagcctggggcctcagtgaaggtttcctgcaaggcatctggatacgccttcaccagctactatatgcactgggcgcgacaggcccctggacaagggcttgagtggatgggaataatcaaccctaggggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 382 - protein, 116 aa, Homo sapiens; identical to SEQ ID NO: 232
  Antibody 39

SEQ ID NO: 383 - protein, 5 aa, Homo sapiens
  Antibody 39
SYYMH

SEQ ID NO: 384 - protein, 17 aa, Homo sapiens
  Antibody 39
IINPRGGSTSYAQKFQG

SEQ ID NO: 385 - protein, 7 aa, Homo sapiens
  Antibody 39
GKYWMYD

SEQ ID NO: 386 - DNA, 333 bp, Homo sapiens
  Antibody 39
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacggcgtgggagtggccgttcggaactgggaccaagctgaccgtccta

SEQ ID NO: 387 - protein, 111 aa, Homo sapiens
  Antibody 39
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 388 - protein, 13 aa, Homo sapiens
  Antibody 39
SGGSSNIGNSYVS

SEQ ID NO: 389 - protein, 7 aa, Homo sapiens
  Antibody 39
DNNKRPS

SEQ ID NO: 390 - protein, 12 aa, Homo sapiens
  Antibody 39
GTWDTSTAWEWP

SEQ ID NO: 391 - DNA, 348 bp, Homo sapiens; identical to SEQ ID NO: 51
  Antibody 40

SEQ ID NO: 392 - protein, 116 aa, Homo sapiens; identical to SEQ ID NO: 52
  Antibody 40

SEQ ID NO: 393 - protein, 5 aa, Homo sapiens
  Antibody 40
SYYMH

SEQ ID NO: 394 - protein, 17 aa, Homo sapiens
  Antibody 40
IINPSGGSTSYAQKFQG

SEQ ID NO: 395 - protein, 7 aa, Homo sapiens
  Antibody 40
GKYWMYD

SEQ ID NO: 396 - DNA, 333 bp, Homo sapiens; identical to SEQ ID NO: 356
  Antibody 40

SEQ ID NO: 397 - protein, 111 aa, Homo sapiens; identical to SEQ ID NO: 357
  Antibody 40

SEQ ID NO: 398 - protein, 13 aa, Homo sapiens
  Antibody 40
SGGSSNIGNSYVS

SEQ ID NO: 399 - protein, 7 aa, Homo sapiens
  Antibody 40
DNNKRPS

SEQ ID NO: 400 - protein, 12 aa, Homo sapiens
  Antibody 40
GTWDSSTVWEWP

SEQ ID NO: 401 - DNA, 348 bp, Homo sapiens
  Antibody 41
caggtccagctggtgcagtctggggctgaggtgaggaagcctggggcctcagtgaaggtttcctgcaaggcatctggatacgccttcaccagctactatatgcactgggcgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagacctgaggacacggccgtgtattactgtgcgagaggaaagtactggatgtacgactggggcaaaggcaccctggtcaccgtctcgggt

SEQ ID NO: 402 - protein, 116 aa, Homo sapiens
  Antibody 41
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 403 - protein, 5 aa, Homo sapiens
  Antibody 41
SYYMH

SEQ ID NO: 404 - protein, 17 aa, Homo sapiens
  Antibody 41
IINPSGGSTSYAQKFQG

SEQ ID NO: 405 - protein, 7 aa, Homo sapiens
  Antibody 41
GKYWMYD

SEQ ID NO: 406 - DNA, 333 bp, Homo sapiens
  Antibody 41
cagtctgtgttgacgcagccgccctcagtgtctgcggccccaggacagaaggtcaccatctcctgctctggaggcagcaccaacattgggaatagttatgtatcctggtaccagcgactcccaggaacagcccccaaactcctcatttacgacaataataagcgacccccagggattcctgaccgattctctggctccaagtctggcacgtcagccaccctggccatcaccggactccagactggggacgaggccgattattactgcggaacatgggataccagcacggtgtgggagtggccgttcggaactgggaccaagctgaccgtccta

SEQ ID NO: 407 - protein, 111 aa, Homo sapiens
  Antibody 41
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 408 - protein, 13 aa, Homo sapiens
  Antibody 41
SGGSTNIGNSYVS

SEQ ID NO: 409 - protein, 7 aa, Homo sapiens
  Antibody 41
DNNKRPP

SEQ ID NO: 410 - protein, 12 aa, Homo sapiens
  Antibody 41
GTWDTSTVWEWP

SEQ ID NO: 411 - DNA, 348 bp, Homo sapiens
  Antibody 42
caggtccagctggtgcagtctggggctgaggtgaagaagcctggggcctcagtgaaggtttcctgcaaggcatctggatacgcctttaccagctactatatgcactgggcgcgacaggcccctggacaggggcttgagtgggtgggaataatcaaccctagcggtggtagcacaagctacgcacagaagttccagggcagagtcaccatgaccagggacacgtccacgagcacagtctacatggagctgagcagcctgagatctggggacacggccgtgtattactgtgcgagaggaaagtactggatgtacgactggggcaaaggcaccctggtcaccgtctcgagt

SEQ ID NO: 412 - protein, 116 aa, Homo sapiens
  Antibody 42
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 413 - protein, 5 aa, Homo sapiens
  Antibody 42
SYYMH

SEQ ID NO: 414 - protein, 17 aa, Homo sapiens
  Antibody 42
IINPSGGSTSYAQKFQG

SEQ ID NO: 415 - protein, 7 aa, Homo sapiens
  Antibody 42
GKYWMYD

SEQ ID NO: 416 - DNA, 333 bp, Homo sapiens
  Antibody 42
caggctgtgttgacgcagccgccctcagtgtctgcggccccaggacagaaggtcaccatctcctgctctggaggcagctccaacattgggaatagttatgtatcctggtaccagcgactcccaggagcagcccccaaactcctcatttacgacaataataagcgaccctcagggattcctgaccgattctctggctccaagtctggcacgtcagccaccctggccatcaccggactccagactggggacgaggccgattattactgcggaacatgggataccagcacggggtgggagtggccgttcggaactgggaccaagctgaccgtccta

SEQ ID NO: 417 - protein, 111 aa, Homo sapiens
  Antibody 42
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 418 - protein, 13 aa, Homo sapiens
  Antibody 42
SGGSSNIGNSYVS

SEQ ID NO: 419 - protein, 7 aa, Homo sapiens
  Antibody 42
DNNKRPS

SEQ ID NO: 420 - protein, 12 aa, Homo sapiens
  Antibody 42
GTWDTSTGWEWP

SEQ ID NO: 421 - DNA, 348 bp, Homo sapiens
  Antibody 24 PGL
caggtccagctggtgcagtctggggctgaggtgaagaagcctggggcctcagtgaaggtttcctgcaaggcatctggatacgccttcaccagctactatatgcactgggtgcgacaggcccctggacaagggcttgagtggatgggaataatcaaccctagaggtggtagcacaagctacgcacagaagttccagggcagagtcaccatgaccagggacacgtccacgagcacagtctacatggagctgagcagcctgagatctgaggacacggccgtgtattactgtgcgagaggaaagtactggatgtacgactggggcaaaggcaccctggtcaccgtctcgagt

SEQ ID NO: 422 - protein, 116 aa, Homo sapiens
  Antibody 24 PGL
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 423 - protein, 5 aa, Homo sapiens
  Antibody 24 PGL
SYYMH

SEQ ID NO: 424 - protein, 17 aa, Homo sapiens
  Antibody 24 PGL
IINPRGGSTSYAQKFQG

SEQ ID NO: 425 - protein, 7 aa, Homo sapiens
  Antibody 24 PGL
GKYWMYD

SEQ ID NO: 426 - DNA, 333 bp, Homo sapiens
  Antibody 24 PGL
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctgggcatcaccggactccagactggggacgaggccgattatttctgcggaacatgggataccagcacggtgtgggagtggccgttcggaactgggaccaagctgaccgtccta

SEQ ID NO: 427 - protein, 111 aa, Homo sapiens
  Antibody 24 PGL
QSVLTQPPSVSAAPGQKVTISCSGGSSNIGNSYVSWYQQLPGTAPKLLIYDNNKRPSGIPDRFSGSKSGTSATLGITGLQTGDEADYFCGTWDTSTVWEWPFGTGTKLTVL

SEQ ID NO: 428 - protein, 13 aa, Homo sapiens
  Antibody 24 PGL
SGGSSNIGNSYVS

SEQ ID NO: 429 - protein, 7 aa, Homo sapiens
  Antibody 24 PGL
DNNKRPS

SEQ ID NO: 430 - protein, 12 aa, Homo sapiens
  Antibody 24 PGL
GTWDTSTVWEWP

SEQ ID NO: 431 - DNA, 348 bp, Homo sapiens; identical to SEQ ID NO: 421
  Antibody 24 GL

SEQ ID NO: 432 - protein, 116 aa, Homo sapiens; identical to SEQ ID NO: 422
  Antibody 24 GL

SEQ ID NO: 433 - protein, 5 aa, Homo sapiens
  Antibody 24 GL
SYYMH

SEQ ID NO: 434 - protein, 17 aa, Homo sapiens
  Antibody 24 GL
IINPRGGSTSYAQKFQG

SEQ ID NO: 435 - protein, 7 aa, Homo sapiens
  Antibody 24 GL
GKYWMYD

SEQ ID NO: 436 - DNA, 333 bp, Homo sapiens
  Antibody 24 GL
cagtctgtgttgacgcagccgccctcagtgtctgcggccccaggacagaaggtcaccatctcctgctctggaggcagctccaacattgggaatagttatgtatcctggtaccagcaactcccaggaacagcccccaaactcctcatttacgacaataataagcgaccctcagggattcctgaccgattctctggctccaagtctggcacgtcagccaccctgggcatcaccggactccagactggggacgaggccgattattactgcggaacatgggataccagcacggtgtgggagtggccgttcggaactgggaccaagctgaccgtccta

SEQ ID NO: 437 - protein, 111 aa, Homo sapiens
  Antibody 24 GL
QSVLTQPPSVSAAPGQKVTISCSGGSSNIGNSYVSWYQQLPGTAPKLLIYDNNKRPSGIPDRFSGSKSGTSATLGITGLQTGDEADYYCGTWDTSTVWEWPFGTGTKLTVL

SEQ ID NO: 438 - protein, 13 aa, Homo sapiens
  Antibody 24 GL
SGGSSNIGNSYVS

SEQ ID NO: 439 - protein, 7 aa, Homo sapiens
  Antibody 24 GL
DNNKRPS

SEQ ID NO: 440 - protein, 12 aa, Homo sapiens
  Antibody 24 GL
GTWDTSTVWEWP

SEQ ID NO: 441 - DNA, 348 bp, Homo sapiens; identical to SEQ ID NO: 421
  Antibody 37 GL

SEQ ID NO: 442 - protein, 116 aa, Homo sapiens; identical to SEQ ID NO: 422
  Antibody 37 GL

SEQ ID NO: 443 - protein, 5 aa, Homo sapiens
  Antibody 37 GL
SYYMH

SEQ ID NO: 444 - protein, 17 aa, Homo sapiens
  Antibody 37 GL
IINPRGGSTSYAQKFQG

SEQ ID NO: 445 - protein, 7 aa, Homo sapiens
  Antibody 37 GL
GKYWMYD

SEQ ID NO: 446 - DNA, 333 bp, Homo sapiens
  Antibody 37 GL
cagtctgtgttgacgcagccgccctcagtgtcagcggccccaggacagaaggtcaccatctcctgctctggaggcggctccagcattgggaatagctatgtatcctggtaccagcaactcccaggaacagcccccaaactcctcatctacgacaataataagcgaccctcagggattcctgaccgattctctggctccaagtctggcacgtcagccaccctgggcatcaccggactccagactggggacgaggccgattattactgcggaacatgggataccagcccggtgtgggagtggccgttcggaactgggaccaagctgaccgtccta

SEQ ID NO: 447 - protein, 111 aa, Homo sapiens
  Antibody 37 GL
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 448 - protein, 13 aa, Homo sapiens
  Antibody 37 GL
SGGGSSIGNSYVS

SEQ ID NO: 449 - protein, 7 aa, Homo sapiens
  Antibody 37 GL
DNNKRPS

SEQ ID NO: 450 - protein, 12 aa, Homo sapiens
  Antibody 37 GL
GTWDTSPVWEWP

SEQ ID NO: 451 - DNA, 96 bp, Artificial Sequence
  Description of Artificial Sequence Synthetic Cyno IL4R primer 1
ggggacaagtttgtacaaaaaagcaggcttctttaactttaagaaggagatataaccatggggtggctttgctctgggctcctgttgcctgtgagc

SEQ ID NO: 452 - DNA, 63 bp, Artificial Sequence
  Description of Artificial Sequence Synthetic Cyno IL4R primer 2
ggggaccactttgtacaagaaagctgggtcctgctcgaagggctccctgtaggagttgtacca

SEQ ID NO: 453 - DNA, 32 bp, Artificial Sequence
  Description of Artificial Sequence Synthetic I75V oligonucleotide mutation primer
gaagcccacacgtgtgccctgagaacaacgga

SEQ ID NO: 454 - protein, 478 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFDPAFLYKVVGAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 455 - DNA, 693 bp, Macaca fascicularis
  Cynomolgus monkey IL-4Ra cDNA nucleotide
atggggtggctttgctctgggctcctgttgcctgtgagctgcctggtcctgctgcaggtggcaagctctgggagcatgaaggtcctgcaggagcccacctgcgtctccgactacatgagcatctctacctgtgagtggaagatgggcggtcccaccaattgcagcgccgagctccgtctgttgtaccagctggtttttcagtcctccgaaacccacacgtgtgtccccgagaacaacggcggtgtggggtgcgtgtgccacctgctcatggatgatgtggtcagtatggacaactatacgctggacctgtgggctggacagcagctgctgtggaagggctccttcaagcccagcgagcatgtgaaacccagggccccaggaaacctcacggttcacaccaatgtctccgacactgtgctgctgacctggagcaacccatatccccctgacaattacctgtataatgatctcacctatgcagtcaacatttggagtgaaaacgacccggcatattccagaatccataacgtgacctacctaaaacccaccctccgcatcccagccagcaccctgaagtctggaatttcctacagggcacgggtgagggcctgggctcagcactataacaccacctggagtgagtggagccccagcaccaagtggtacaactcctacagggagcccttcgagcag

SEQ ID NO: 456 - DNA, 1428 bp, Macaca fascicularis
  Cynomolgus monkey IL-4Ra/Fc cDNA nucleotide
atggggtggctttgctctgggctcctgttgcctgtgagctgcctggtcctgctgcaggtggcaagctctgggagcatgaaggtcctgcaggagcccacctgcgtctccgactacatgagcatctctacctgtgagtggaagatgggcggtcccaccaattgcagcgccgagctccgtctgttgtaccagctggtttttcagtcctccgaaacccacacgtgtgtccccgagaacaacggcggtgtggggtgcgtgtgccacctgctcatggatgatgtggtcagtatggacaactatacgctggacctgtgggctggacagcagctgctgtggaagggctccttcaagcccagcgagcatgtgaaacccagggccccaggaaacctcacggttcacaccaatgtctccgacactgtgctgctgacctggagcaacccatatccccctgacaattacctgtataatgatctcacctatgcagtcaacatttggagtgaaaacgacccggcatattccagaatccataacgtgacctacctaaaacccaccctccgcatcccagccagcaccctgaagtctggaatttcctacagggcacgggtgagggcctgggctcagcactataacaccacctggagtgagtggagccccagcaccaagtggtacaactcctacagggagcccttcgagcaggacccagctttcttgtacaaagtggttcgattcgaggagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaaaaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 457 - protein, 475 aa, Macaca fascicularis
  Cynomolgus monkey IL-4Ra/Fc protein
MGWLCSGLLLPVSCLVLLQVASSGSMKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQDPAFLYKVVRFEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - DNA, 1422 bp, Homo sapiens
  Human I75V IL-4Ra/Fc cDNA nucleotide
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtgtccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgacccagctttcttgtacaaagtggttcgattcgaggagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaaaaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 459 - protein, 473 aa, Homo sapiens
  Human I75V IL-4Ra/Fc protein
MGWLCSGLLFPVSCLVLLQVASSGNMKVLQEPTCVSDYMSISTCEWKMNGPTNCSTELRLLYQLVFLLSEAHTCVPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFDPAFLYKVVRFEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 460 - protein, 261 aa, Homo sapiens
  Human IL-4Ra/HIS tag polypeptide
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFNPAFLYKVVGAADYKDDDDKAAHHHHHHHHHH

SEQ ID NO: 461 - protein, 262 aa, Mus musculus
  Murine IL-4Ra/HIS tag polypeptide
MGRLCTKFLTSVGCLILLLVTGSGSIKVLGEPTCFSDYIRTSTCEWFLDSAVDCSSQLCLHYRLMFFEFSENLTCIPRNSASTVCVCHMEMNRPVQSDRYQMELWAEHRQLWQGSFSPSGNVKPLAPDNLTLHTNVSDEWLLTWNNLYPSNNLLYKDLISMVNISREDNPAEFIVYNVTYKEPRLSFPINILMSGVYYTARVRVRSQILTGTWSEWSPSITWYNHFQLPLNPAFLYKVVGAADYKDDDDKAAHHHHHHHHHH